SEQUENCE LISTING (3 sequences)

SEQ ID NO: 1 - protein, 218 aa, Homo sapiens
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, Homo sapiens
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

(19) United States
(12) Patent Application Publication
(10) Pub. No.: US 2026/0207760 A1
(43) Pub. Date: Jul. 23, 2026

(54) COMBINATIONS OF CD33 ANTIBODY DRUG CONJUGATE WITH CHEMOTHERAPEUTIC AGENTS

(71) Applicant: Glykos Biomedical Oy, Helsinki (FI)

(72) Inventors: Tero Satomaa, Helsinki (FI); Juhani Saarinen, Helsinki (FI); Jukka Hiltunen, Helsinki (FI)

(73) Assignee: Glykos Biomedical Oy, Helsinki (FI)

(21) Appl. No.: 19/138,969

(22) PCT Filed: Dec. 17, 2023
(86) PCT No.: PCT/IB2023/062846

§ 371 Date: Jun. 13, 2025

Related U.S. Application Data
(60) Provisional application No. 63/433,525, filed on Dec. 19, 2022.

(51) Int. Cl. A61K 47/68; A61K 31/496; A61K 31/497; A61K 31/553; A61K 31/704; A61K 31/7068; A61P 35/02; C07K 16/28

(52) U.S. Cl. CPC A61K 47/68031; A61K 31/496; A61K 31/497; A61K 31/553; A61K 31/704; A61K 31/7068; A61K 47/6849; A61K 47/6889; A61P 35/02; C07K 16/2803

(57) ABSTRACT

This invention relates to the treatment of cancer using a CD33 antibody drug conjugate in combination with chemotherapeutic agents.

### RELATED APPLICATIONS

This application is filed under 35 U.S.C. § 371 and claims priority to International Application No. PCT/IB2023/062846 filed on Dec. 17, 2023, and claims priority to U.S. Ser. No. 63/433,525 filed on Dec. 19, 2022, which are hereby entirely incorporated by reference into this application.

### TECHNICAL FIELD

This invention relates to treatment of cancer using a CD33 antibody drug conjugate in combination with chemotherapeutic agents.

### BACKGROUND OF THE DISCLOSURE

CD33 is a 67 kDa plasma membrane protein that binds to sialic acid and is a member of the sialic acid-binding Ig-related lectin (SIGLEC) family of proteins. CD33 is known to be expressed on myeloid cells. CD33 expression has also been reported on a number of malignant cells. Improvements in treatment of CD33 expressing cancers are being sought and the present invention solves these and other problems.

### SUMMARY OF THE DISCLOSURE

It has now been surprisingly found that the combination of chemotherapeutic agent with a CD33-targeted ADC LNAuM has synergistic or additive effects against leukemia cells both in vitro and in vivo as compared with the LNAuM alone and chemotherapeutic agent alone.

The CD33-targeted ADC LNAuM was found to have potent activity against patient-derived AML blasts without off-target lymphocyte toxicity, in contrast to previous ADCs such as gemtuzumab ozogamicin (Mylotarg). Therefore, the combination of LNAuM with chemotherapeutic agent may preferentially target AML blasts and spare normal cells from toxic effects.

In an embodiment, the present invention provides a method of treating CD33 expressing cancer in a subject comprising administering to the subject an effective amount of chemotherapeutic agent and an effective amount of LNAuM of Formula (I):

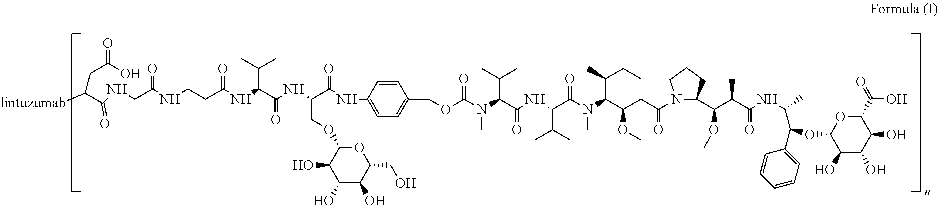

wherein
   n is an integer from 1 to 20.

In an embodiment, the present invention provides a method of treating CD33 expressing cancer in a subject comprising administering to the subject an effective amount of chemotherapeutic agent and an effective amount of LNAuM of Formula (I):

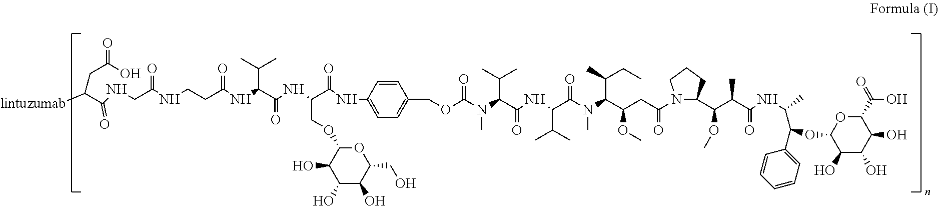

wherein
   lintuzumab comprises a light chain having the amino acid sequence set forth in SEQ ID NO:1 and a heavy chain having the amino acid sequence set forth in SEQ ID NO:2, and
   n is an integer from 1 to 20.

The present invention further provides for LNAuM of Formula (I)

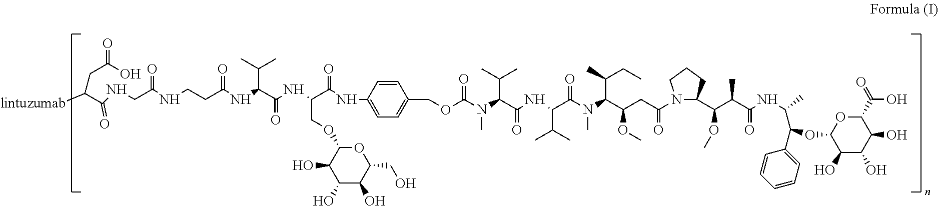

for use in a method for treating CD33 expressing cancer in a subject, wherein the method comprises administering to the subject an effective amount of the LNAuM and an effective amount of a chemotherapeutic agent, and wherein
lintuzumab comprises a light chain having the amino acid sequence set forth in SEQ ID NO: 1 and a heavy chain having the amino acid sequence set forth in SEQ ID NO:2, and
n is an integer from 1 to 20.

The present invention further provides for LNAuM of Formula (I)

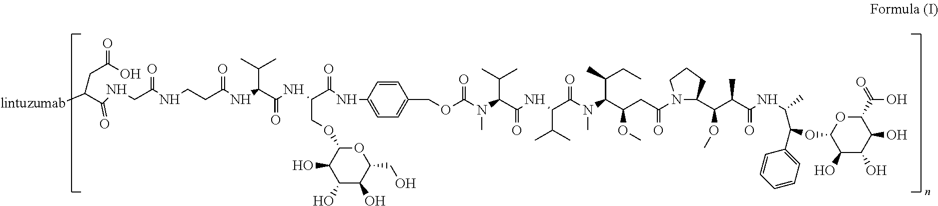

and a chemotherapeutic agent as a combination for use in a method for treating CD33 expressing cancer in a subject, wherein the method comprises administering to the subject an effective amount of the LNAuM and an effective amount of the chemotherapeutic agent, and wherein
lintuzumab comprises a light chain having the amino acid sequence set forth in SEQ ID NO: 1 and a heavy chain having the amino acid sequence set forth in SEQ ID NO:2, and
n is an integer from 1 to 20.

In an embodiment, the cancer is a hematologic cancer.

In an embodiment, the hematologic cancer is leukemia.

In an embodiment, the leukemia is selected from the group consisting of acute myeloid leukemia (AML), chronic myeloid leukemia (CML), acute lymphoblastic leukemia (ALL), myelodysplastic syndrome (MDS), and acute promyelocytic leukaemia (APL).

In an embodiment, the cancer is acute myeloid leukemia (AML).

In an embodiment, the acute myeloid leukemia (AML) is refractory or relapse acute myeloid leukemia.

In an embodiment, the acute myeloid leukemia (AML) is characterized by FLT3 internal tandem duplication (FLT3-ITD).

In an embodiment, the chemotherapeutic agent is selected from the group consisting of an FLT3 inhibitor and a BCL2 inhibitor.

In an embodiment, the FLT3 inhibitor is selected from the group consisting of midostaurin and gilteritinib fumarate.

In an embodiment, the BCL2 inhibitor is venetoclax.

In an embodiment, the chemotherapeutic agent is selected from the group consisting of cytarabine and daunorubicin.

In an embodiment, the chemotherapeutic agent is venetoclax.

In an embodiment, venetoclax is administered at a daily dose of at least 50 mg, at least 70 mg, at least 100 mg, or at least 200 mg.

In an embodiment, the chemotherapeutic agent is cytarabine.

In an embodiment, cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

In an embodiment, the chemotherapeutic agent is midostaurin.

In an embodiment, midostaurin is administered daily at least a dose of 30 mg/m², 45 mg/m² or 60 mg/m².

In an embodiment, the chemotherapeutic agent is gilteritinib.

In an embodiment, gilteritinib is administered at a dose at least about 5 mg/day, at least about 10 mg/day, at least about 40 mg/day, or at least about 80 mg/day, or at least about 120 mg/day.

In an embodiment, the chemotherapeutic agent is daunorubicin.

In an embodiment, daunorubicin is administered at a dose at least about 10 mg/m²/day, at least about 30 mg/m²/day, at least about 60 mg/m²/day, or at least about 90 mg/m²/day.

In an embodiment, LNAuM is administered at a dose of at least about 0.1 mg/kg, at least about 0.3 mg/kg, at least about 0.5 mg/kg, at least about 0.8 mg/kg, at least about 1 mg/kg, at least about 1.5 mg/kg, at least about 2 mg/kg, at least about 2.5 mg/kg, at least about 3 mg/kg, at least about 3.5 mg/kg, or at least about 4 mg/kg.

In an embodiment, the present invention provides a method of treating CD33 expressing acute myeloid leukemia (AML) in a subject comprising a step of administering cytarabine and LNAuM of Formula (I),

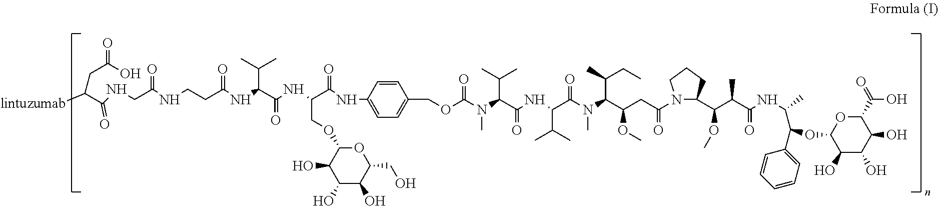

wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

In an embodiment, the present invention provides a method of treating CD33 expressing acute myeloid leukemia (AML) in a subject comprising the step of administering venetoclax and LNAuM of Formula (I),

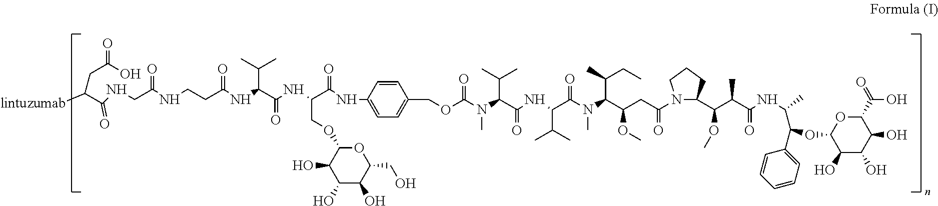

wherein venetoclax is administered at a daily dose of least 10 mg, at least 20 mg, at least 50 mg, at least 100 mg, at least 200 mg or at least 400 mg; and
n is an integer from 1 to 10.

In an embodiment, the present invention provides a method of treating CD33 expressing acute myeloid leukemia (AML) in a subject comprising the step of administering midostaurin and LNAuM of Formula (I),

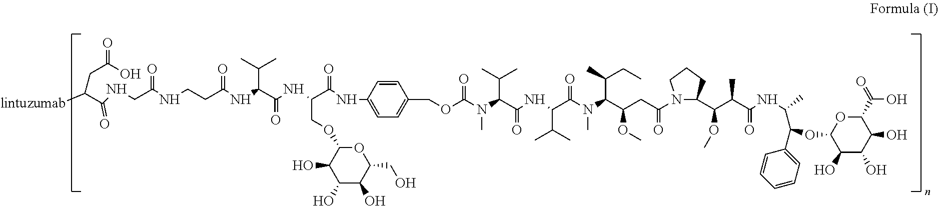

wherein midostaurin is administered daily at least a dose of 30 mg/m², 45 mg/m² or 60 mg/m²; and
n is an integer from 1 to 10.

In an embodiment, the present invention provides a method of treating CD33 expressing acute myeloid leukemia (AML) in a subject comprising the step of administering gilteritinib and LNAuM of Formula (I),

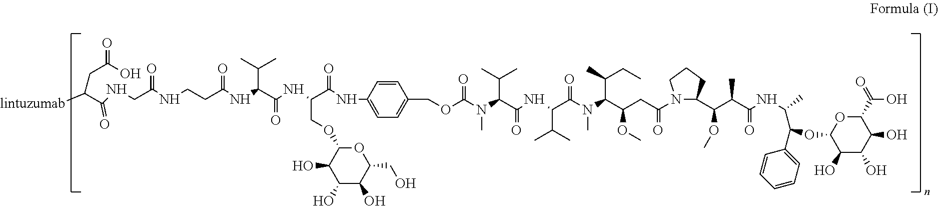

wherein gilteritinib is administered at a dose at least about 5 mg/day, at least about 10 mg/day, at least about 40 mg/day, or at least about 80 mg/day, or at least about 120 mg/day; and
n is an integer from 1 to 10.

In an embodiment, the present invention provides a method of treating CD33 expressing acute myeloid leukemia (AML) in a subject comprising a step of administering daunorubicin and LNAuM of Formula (I),

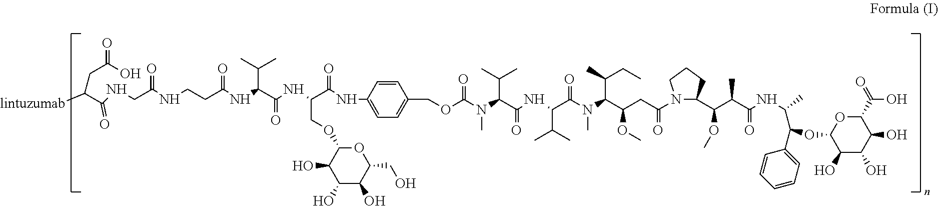

wherein daunorubicin is administered at a dose at least about 10 mg/m²/day, at least about 30 mg/m²/day, at least about 60 mg/m²/day, or at least about 90 mg/m²/day; and
n is an integer from 1 to 10.

In an embodiment, the present invention provides a method of treating CD33 expressing acute myeloid leukemia (AML) in a subject comprising a step of administering cytarabine, venetoclax and LNAuM of Formula (I),

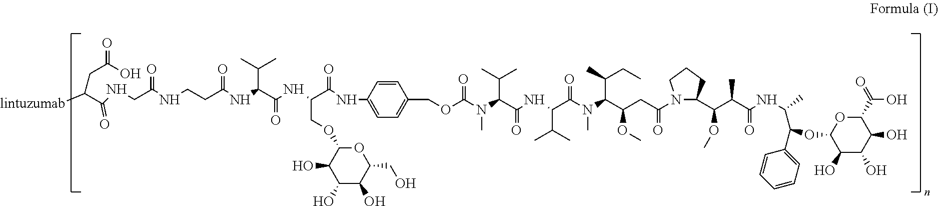

wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered venetoclax is administered at a daily dose of at least 10 mg, at least 20 mg, at least 50 mg, at least 100 mg, at least 200 mg or at least 400 mg; and
n is an integer from 1 to 10.

In an embodiment, LNAuM of Formula (I) for use in a method for treating CD33 expressing acute myeloid leukemia (AML) in a subject is disclosed

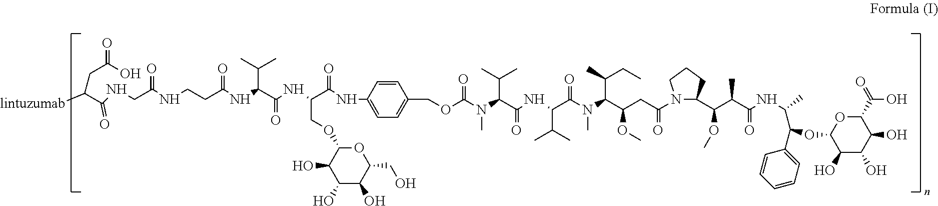

wherein the method comprises a step of administering cytarabine and the LNAuM, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

In an embodiment, LNAuM of Formula (I) for use in a method for treating CD33 expressing acute myeloid leukemia (AML) in a subject is disclosed

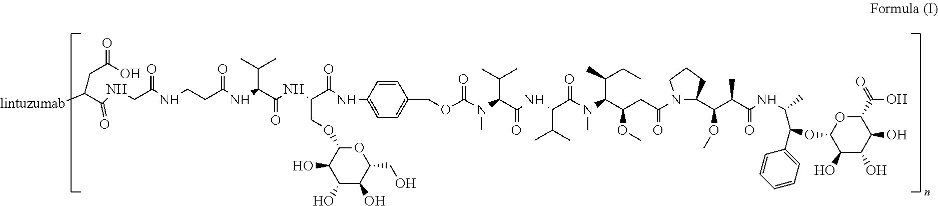

wherein the method comprises the step of administering venetoclax and the LNAuM, wherein venetoclax is administered at a daily dose of least 10 mg, at least 20 mg, at least 50 mg, at least 100 mg, at least 200 mg or at least 400 mg; and
n is an integer from 1 to 10.

In an embodiment, LNAuM of Formula (I) for use in a method for treating CD33 expressing acute myeloid leukemia (AML) in a subject is disclosed

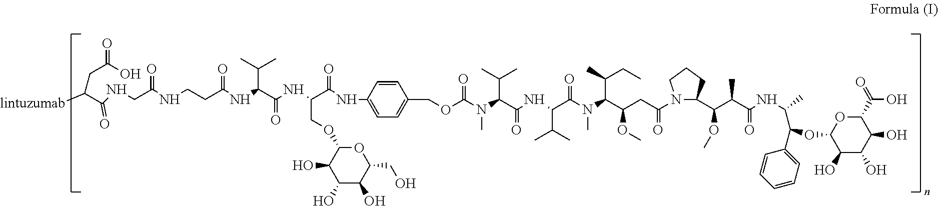

wherein the method comprises the step of administering midostaurin and LNAuM of Formula (I), wherein midostaurin is administered daily at least a dose of 30 mg/m², 45 mg/m² or 60 mg/m²; and
n is an integer from 1 to 10.

In an embodiment, LNAuM of Formula (I) for use in a method for treating CD33 expressing acute myeloid leukemia (AML) in a subject is disclosed

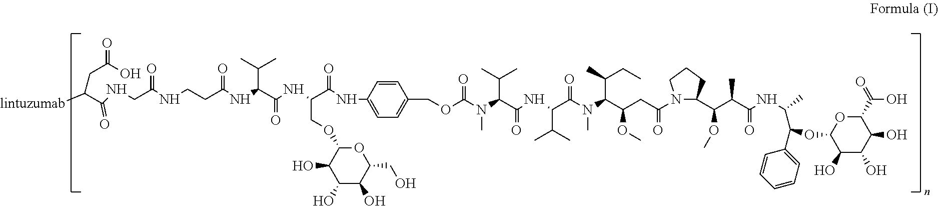

wherein the method comprises the step of administering gilteritinib and the LNAuM, wherein gilteritinib is administered at a dose at least about 5 mg/day, at least about 10 mg/day, at least about 40 mg/day, or at least about 80 mg/day, or at least about 120 mg/day; and
n is an integer from 1 to 10.

In an embodiment, LNAuM of Formula (I) for use in a method for treating CD33 expressing acute myeloid leukemia (AML) in a subject is disclosed

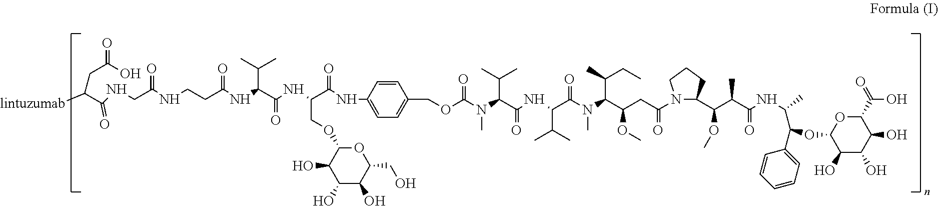

wherein the method comprises a step of administering daunorubicin and the LNAuM,
wherein daunorubicin is administered at a dose at least about 10 mg/m²/day, at least about 30 mg/m²/day, at least about 60 mg/m²/day, or at least about 90 mg/m²/day; and
n is an integer from 1 to 10.

In an embodiment, LNAuM of Formula (I) for use in a method for treating CD33 expressing acute myeloid leukemia (AML) in a subject is disclosed

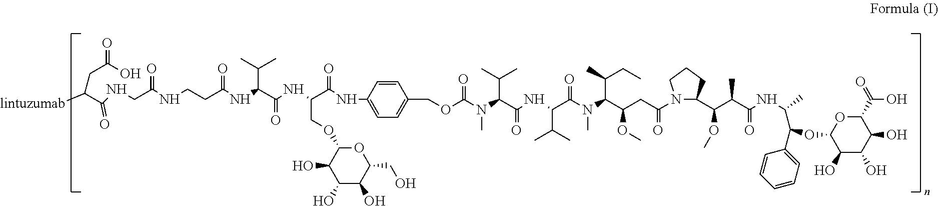

wherein the method comprises a step of administering cytarabine, venetoclax, and the LNAuM, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered at a daily dose of at least 10 mg, at least 20 mg, at least 50 mg, at least 100 mg, at least 200 mg or at least 400 mg; and
n is an integer from 1 to 10.

In an embodiment, LNAuM of Formula (I) and cytarabine as a combination for use in a method for treating CD33 expressing acute myeloid leukemia (AML) in a subject is disclosed

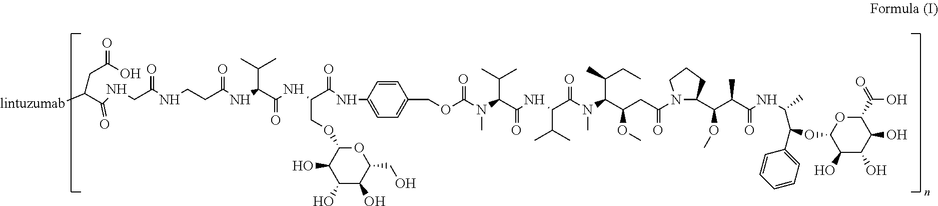

wherein the method comprises a step of administering cytarabine and the LNAuM,
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

In an embodiment, LNAuM of Formula (I) and venetoclax as a combination for use in a method for treating CD33 expressing acute myeloid leukemia (AML) in a subject is disclosed

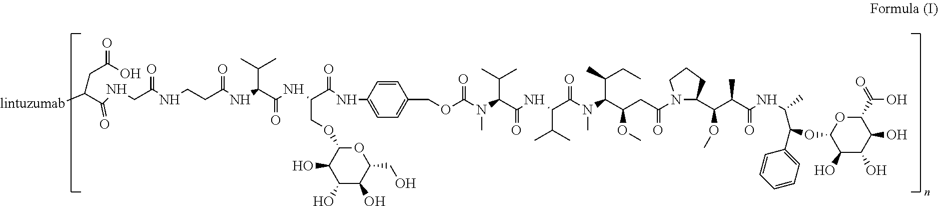

wherein the method comprises the step of administering venetoclax and the LNAuM,
wherein venetoclax is administered at a daily dose of least 10 mg, at least 20 mg, at least 50 mg, at least 100 mg, at least 200 mg or at least 400 mg; and
n is an integer from 1 to 10.

In an embodiment, LNAuM of Formula (I) and midostaurin as a combination for use in a method for treating CD33 expressing acute myeloid leukemia (AML) in a subject is disclosed

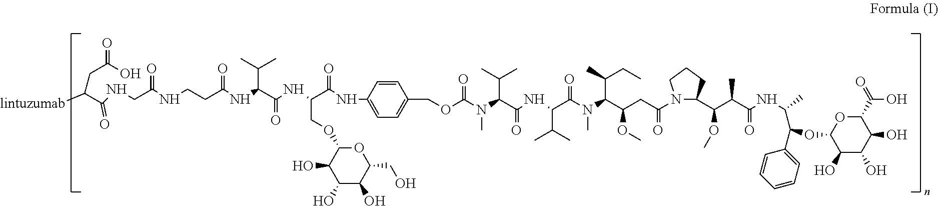

wherein the method comprises the step of administering midostaurin and the LNAuM,
wherein midostaurin is administered daily at least a dose of 30 mg/m², 45 mg/m² or 60 mg/m²; and
n is an integer from 1 to 10.

In an embodiment, LNAuM of Formula (I) and gilteritinib as a combination for use in a method for treating CD33 expressing acute myeloid leukemia (AML) in a subject is disclosed

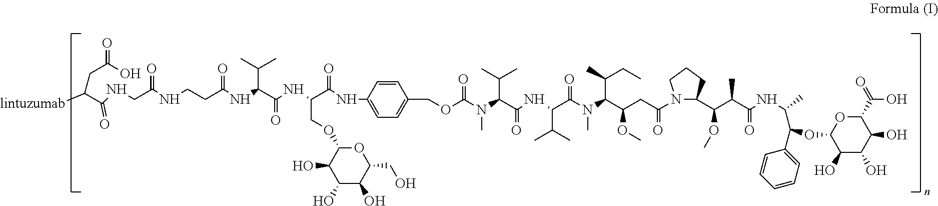

wherein the method comprises the step of administering gilteritinib and the LNAuM,
wherein gilteritinib is administered at a dose at least about 5 mg/day, at least about 10 mg/day, at least about 40 mg/day, or at least about 80 mg/day, or at least about 120 mg/day; and
n is an integer from 1 to 10.

In an embodiment, LNAuM of Formula (I) and daunorubicin as a combination for use in a method for treating CD33 expressing acute myeloid leukemia (AML) in a subject is disclosed

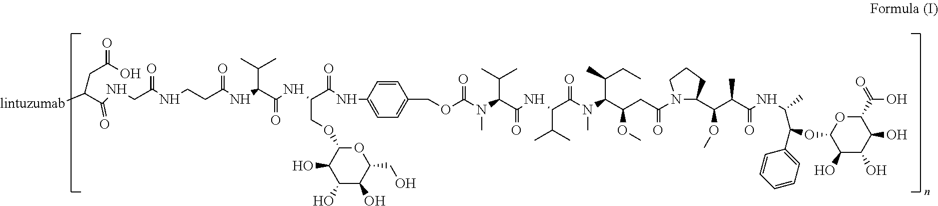

wherein the method comprises a step of administering daunorubicin and the LNAuM,
wherein daunorubicin is administered at a dose at least about 10 mg/m²/day, at least about 30 mg/m²/day, at least about 60 mg/m²/day, or at least about 90 mg/m²/day; and
n is an integer from 1 to 10.

In an embodiment, LNAuM of Formula (I), cytarabine, and venetoclax as a combination for use in a method for treating CD33 expressing acute myeloid leukemia (AML) in a subject is disclosed

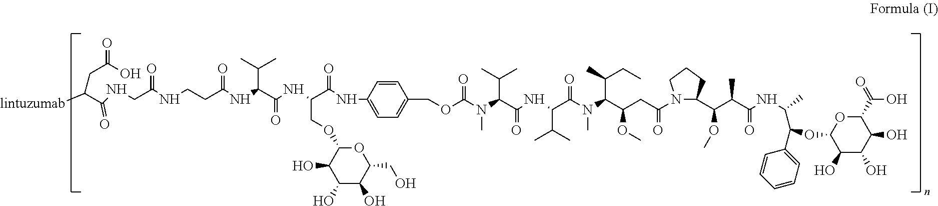

wherein the method comprises a step of administering cytarabine, venetoclax, and the LNAuM,
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered at a daily dose of at least 10 mg, at least 20 mg, at least 50 mg, at least 100 mg, at least 200 mg or at least 400 mg; and
n is an integer from 1 to 10.

The term “combination” may be understood broadly. The chemotherapeutic agent and the LNAuM may be provided as separate and/or distinct compositions, which are administered simultaneously and/or sequentially. The chemotherapeutic agent and the LNAuM may be copresented in separate packaging, or they may be separately packaged and available for sale independently of one another. They may however be co-marketed or co-promoted for simultaneous and/or sequential administration. In some embodiments, the chemotherapeutic agent and the LNAuM may be provided as a single composition comprising both the chemotherapeutic agent and the LNAuM.

Other embodiments are also disclosed herein which will be apparent to those of ordinary skill in the art.

### BRIEF DESCRIPTION OF THE DRAWINGS

FIG. 1 shows the viability of HL-60 cells (% of control) after 4 days treatment with LNAuM and cytarabine. FIG. 1A shows 30 pM LNAuM with cytarabine, FIG. 1B shows 100 pM LNAuM with cytarabine, FIG. 1C shows 300 pM LNAuM with cytarabine, and FIG. 1D shows 1000 pM LNAuM with cytarabine; error bars show standard deviation; the data were analysed using one-way ANOVA, Tukey multiple comparison; *, both LNAuM and cytarabine are statistically significant p<0.05 as compared to LNAuM & cytarabine combination; **, both LNAuM and cytarabine are statistically significant p<0.01 as compared to LNAuM & cytarabine combination; and ***, both LNAuM and cytarabine are statistically significant p<0.001 as compared to LNAuM & cytarabine combination.

FIG. 2 shows the viability of HL-60 cells (% of control) after 4 days treatment with LNAuM and venetoclax. FIG. 2A shows 30 pM LNAuM with venetoclax, FIG. 2B shows 100 pM LNAuM with venetoclax, FIG. 2C shows 300 pM LNAuM with venetoclax, and FIG. 2D shows 1000 pM LNAuM with venetoclax; error bars show standard deviation; the data were analysed using one-way ANOVA, Tukey multiple comparison; *, both LNAuM and venetoclax are statistically significant p<0.05 as compared to LNAuM & venetoclax combination; **, both LNAuM and venetoclax are statistically significant p<0.01 as compared to LNAuM & venetoclax combination; and ***, both LNAuM and venetoclax are statistically significant p<0.001 as compared to LNAuM & venetoclax combination.

FIG. 3 shows the viability of HL-60 cells (% of control) after 4 days treatment with LNAuM and daunorubicin. FIG. 3A shows 30 PM LNAuM with daunorubicin, FIG. 3B shows 100 pM LNAuM with daunorubicin, FIG. 3C shows 300 pM LNAuM with daunorubicin, and FIG. 3D shows 1000 pM LNAuM with daunorubicin; error bars show standard deviation; error bars show standard deviations; the data were analysed using one-way ANOVA, Tukey multiple comparison; *, both LNAuM and daunorubicin are statistically significant p<0.05 as compared to LNAuM & daunorubicin combination; **, both LNAuM and daunorubicin are statistically significant p<0.01 as compared to LNAuM & daunorubicin combination; and ***, both LNAuM and daunorubicin are statistically significant p<0.001 as compared to LNAuM & daunorubicin combination. 12 nM daunorubicin+LNAuM wells show the same values as 12 nM daunorubicin alone indicating a pipetting error.

FIG. 4 shows the viability of MOLM-13 cells (% of control) after 4 days treatment with LNAuM and cytarabine. FIG. 4A shows 1 pM LNAuM with cytarabine, FIG. 4B shows 50 pM LNAuM with cytarabine, FIG. 4C shows 250 pM LNAuM with cytarabine, and FIG. 4D shows 5 nM LNAuM with cytarabine; error bars show standard deviation; the data were analysed using one-way ANOVA, Tukey multiple comparison; *, both LNAuM and cytarabine are statistically significant p<0.05 as compared to LNAuM & cytarabine combination; **, both LNAuM and cytarabine are statistically significant p<0.01 as compared to LNAuM & cytarabine combination; and ***, both LNAuM and cytarabine are statistically significant p<0.001 as compared to LNAuM & cytarabine combination.

FIG. 5 shows the viability of MOLM-13 cells (% of control) after 4 days treatment with LNAuM and venetoclax. FIG. 5A shows 1 pM LNAuM with venetoclax, FIG. 5B shows 50 pM LNAuM with venetoclax, FIG. 5C shows 250 pM LNAuM with venetoclax, and FIG. 5D shows 5 nM LNAuM with venetoclax; error bars show standard deviation; the data were analysed using one-way ANOVA, Tukey multiple comparison; *, both LNAuM and venetoclax are statistically significant p<0.05 as compared to LNAuM & venetoclax combination; **, both LNAuM and venetoclax are statistically significant p<0.01 as compared to LNAuM & venetoclax combination; and ***, both LNAuM and venetoclax are statistically significant p<0.001 as compared to LNAuM & venetoclax combination.

FIG. 6 shows the viability of MOLM-13 cells (% of control) after 4 days treatment with LNAuM and midostaurin. FIG. 6A shows 1 pM LNAuM with midostaurin, FIG. 6B shows 50 pM LNAuM with midostaurin, FIG. 6C shows 250 pM LNAuM with midostaurin, and FIG. 6D shows 5 nM LNAuM with midostaurin; error bars show standard deviation; the data were analysed using one-way ANOVA, Tukey multiple comparison; *, both LNAuM and midostaurin are statistically significant p<0.05 as compared to LNAuM & midostaurin combination; **, both LNAuM and midostaurin are statistically significant p<0.01 as compared to LNAuM & midostaurin combination; and ***, both LNAuM and midostaurin are statistically significant p<0.001 as compared to LNAuM & midostaurin combination.

FIG. 7 shows the viability of MOLM-13 cells (% of control) after 4 days treatment with LNAuM and gilteritinib. FIG. 7A shows 1 pM LNAuM with gilteritinib, FIG. 7B shows 50 pM LNAuM with gilteritinib, FIG. 7C shows 250 pM LNAuM with gilteritinib, and FIG. 7D shows 5 nM LNAuM with gilteritinib; error bars show standard deviation; the data were analysed using one-way ANOVA, Tukey multiple comparison; *, both LNAuM and gilteritinib are statistically significant p<0.05 as compared to LNAuM & gilteritinib combination; **, both LNAuM and gilteritinib are statistically significant p<0.01 as compared to LNAuM & gilteritinib combination; and ***, both LNAuM and gilteritinib are statistically significant p<0.001 as compared to LNAuM & gilteritinib combination.

FIG. 8 shows tumor growth in MOLM-13 xenograft study. y-axis shows average tumor size of the study groups and γ-axis shows time in days from the subcutaneous (s.c.) tumor inoculation. Error bars show the standard error of the mean. Comparison of vehicle control, venetoclax 100 mg/kg daily dosing of 10 doses is shown. FIG. 8A shows 0.1 mg/kg LNAuM, FIG. 8B shows 0.3 mg/kg LNAuM, and FIG. 8C shows 1 mg/kg LNAuM, as well as combination of venetoclax and LNAuM as indicated in the Figures.

FIG. 9 shows tumor growth in MOLM-13 xenograft study as tumor volumes of individual mice at two time points. FIG. 9A provides a comparison of vehicle, LNAuM 0.3 mg/kg, venetoclax 100 mg/kg and the combination of the latter two at 15 days after tumor inoculation. y-axis shows the tumor size for each mouse shown as dots (scatterplots). Box plots for each group are shown. Filled box shows the interquartile range (IQR) between the 25th percentile (Q1) and the 75th percentile (Q3). Whiskers show the minimum (Q1-1.5×IQR) and the maximum (Q3+1.5×IQR). Solid line inside the box shows the median and dashed line shows the mean. FIG. 9B provides a comparison of all the compared study groups at study end on day 54 after tumor inoculation. y-axis shows the tumor size for each mouse shown as dots. x-axis shows each study group as indicated on the axis. Animals sacrificed due to tumor growth or tumor rupture due to growth are shown on the upper row.

FIG. 10A shows IC50 values (absolute) of the compounds against patient blasts. FIG. 10B shows IC50 values (absolute) of the compounds against patient lymphocytes in a 5-day assay measured with flow cytometry.

FIG. 11A shows efficacy of the studied compounds against blasts. FIG. 11B shows efficacy of the studied compounds against lymphocytes in patient-derived AML samples in the 5-day assay. Mylotarg=gemtuzumab ozogamicin (GO).

FIG. 12 shows percentage of cell death induced by venetoclax (100 nM), LNAuM (10 nM), and their combination after 4 days.

FIG. 13 shows the viability of MOLM-13 cells (% of control) after treatment with LNAuM and daunorubicin. FIG. 13A shows 1 pM LNAuM with daunorubicin, FIG. 13B shows 20 pM LNAuM with daunorubicin, FIG. 13C shows 100 pM LNAuM with daunorubicin, and FIG. 13D shows 1 nM LNAuM with daunorubicin; error bars show standard deviation; the data were analysed using one-way ANOVA, Tukey multiple comparison; *, both LNAuM and daunorubicin are statistically significant p<0.05 as compared to LNAuM & daunorubicin combination; **, both LNAuM and daunorubicin are statistically significant p<0.01 as compared to LNAuM & daunorubicin combination; and ***, both LNAuM and daunorubicin are statistically significant p<0.001 as compared to LNAuM & daunorubicin combination.

FIG. 14 shows the viability of KG-1 cells (% of control) after treatment with LNAuM and venetoclax. FIG. 14A shows 10 pM LNAuM with venetoclax, FIG. 14B shows 200 pM LNAuM with venetoclax, FIG. 14C shows 700 pM LNAuM with venetoclax, and FIG. 14D shows 4 nM LNAuM with venetoclax; error bars show standard deviation. The data were analysed using one-way ANOVA, Tukey multiple comparison; *, denotes situation when each of LNAuM and venetoclax are at least statistically significant p<0.05 compared to LNAuM & venetoclax combination; **, denotes situation when each of LNAuM and venetoclax are at least statistically significant p<0.01 compared to LNAuM & venetoclax combination; and ***, denotes situation when each of LNAuM and venetoclax are at least statistically significant p<0.001 compared to LNAuM & venetoclax combination.

FIG. 15 shows the viability of KG-1 cells (% of control) after treatment with LNAuM and cytarabine. FIG. 15A shows 10 pM LNAuM with cytarabine, FIG. 15B shows 500 pM LNAuM with cytarabine, FIG. 15C shows 2 nM LNAuM with cytarabine, and FIG. 15C shows 8 nM LNAuM with cytarabine; error bars show standard deviation. The data were analysed using one-way ANOVA, Tukey multiple comparison; *, denotes situation when each of LNAuM and cytarabine are at least statistically significant p<0.05 compared to LNAuM & cytarabine combination; **, denotes situation when each of LNAuM and cytarabine are at least statistically significant p<0.01 compared to LNAuM & cytarabine combination; and ***, denotes situation when each of LNAuM and cytarabine are at least statistically significant p<0.001 compared to LNAuM & cytarabine combination.

FIG. 16 shows the viability of KG-1 cells (% of control) after treatment with LNAuM and daunorubicin. FIG. 16A shows 10 pM LNAuM with daunorubicin, FIG. 16B shows 800 pM LNAuM with daunorubicin, FIG. 16C shows 4 nM LNAuM with daunorubicin, and FIG. 16D shows 8 nM LNAuM with daunorubicin; error bars show standard deviation. The data were analysed using one-way ANOVA, Tukey multiple comparison; ***, denotes situation when each of LNAuM and daunorubicin are at least statistically significant p<0.001 compared to LNAuM & daunorubicin combination.

FIG. 17 shows viability of KG-1 cells (% of control) after treatment with LNAuM and midostaurin. FIG. 17A shows 10 pM LNAuM with midostaurin, FIG. 17B shows 500 pM LNAuM with midostaurin, FIG. 17C shows 2 nM LNAuM with midostaurin, and FIG. 17D shows 8 nM LNAuM with midostaurin; error bars show standard deviation. The data were analysed using one-way ANOVA, Tukey multiple comparison; *, denotes situation when each of LNAuM and midostaurin are at least statistically significant p<0.05 compared to LNAuM & midostaurin combination; **, denotes situation when each of LNAuM and midostaurin are at least statistically significant p<0.01 compared to LNAuM & midostaurin combination; and ***, denotes situation when each of LNAuM and midostaurin are at least statistically significant p<0.001 compared to LNAuM & midostaurin combination.

FIG. 18 shows the viability of KG-1 cells (% of control) after treatment with LNAuM and gilteritinib. FIG. 18A shows 10 pM LNAuM with gilteritinib, FIG. 18B shows 500 pM LNAuM with gilteritinib, FIG. 18C shows 2 nM LNAuM with gilteritinib, and FIG. 18D shows 8 nM LNAuM with gilteritinib; error bars show standard deviation. The data were analysed using one-way ANOVA, Tukey multiple comparison; *, denotes situation when each of LNAuM and gilteritinib are at least statistically significant p<0.05 compared to LNAuM & gilteritinib combination; **, denotes situation when each of LNAuM and gilteritinib are at least statistically significant p<0.01 compared to LNAuM & gilteritinib combination; and ***, denotes situation when each of LNAuM and gilteritinib are at least statistically significant p<0.001 compared to LNAuM & gilteritinib combination.

### DETAILED DESCRIPTION

“LNAuM” is an antibody-drug-conjugate (ADC) comprising the lintuzumab antibody, conjugated to MMAU, via a cleavable linker. LNAuM may be represented as below:

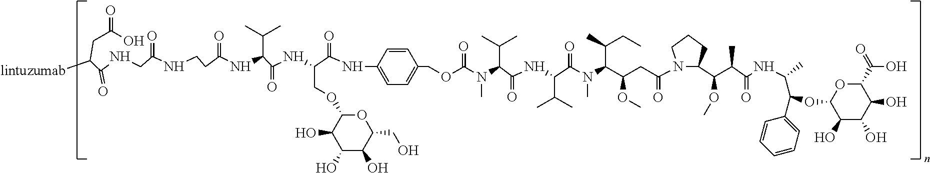

wherein n is an integer from 1 to 20, preferably from 7 to 8.

The term “antibody” is used herein to denote immunoglobulin proteins produced by the body in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab′) 2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein that comprises an antigen-binding site of an antibody and is capable of specifically binding to its antigen.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids.

The term “human CD33” refers to a protein having UniProt ID number P20138.

Expression of CD33 appears to be highly specific to the hematopoietic compartment, with strong expression by myeloid precursor cells. It is expressed by myeloid progenitor cells such as CFU-GEMM, CFU-GM, CFU-G and BFU-E, monocytes/macrophages, granulocyte precursors such as promyelocytes and myelocytes although with decreased expression upon maturation and differentiation, and mature granulocytes though with a low level of expression. Anti-CD33 monoclonal antibodies have shown that CD33 is expressed by clonogenic, acute myelogenous leukemia (AML) cells in greater than 80% of human cases.

The term “CD33 expressing cancer” refers to a cancer characterized by expression of CD33 mRNA and/or protein.

The term “anti-CD33 antibody” refers to an antibody that specifically binds to the human CD33 protein. In the present invention, the anti-CD33 antibody is lintuzumab comprising the light chain of SEQ ID NO:1 and the heavy chain of SEQ ID NO:2. In an embodiment, heavy chain lacks the C-terminal lysine (K446).

TABLE 1
Lintuzumab light and heavy chains.

| | |
|---|---|
| Light chain | DIQMTQSPSS LSASVGDRVT ITCRASESVD |
| | NYGISFMNWF QQKPGKAPKL LIYAASNQGS |
| | GVPSRFSGSG SGTDFTLTIS SLQPDDFATY |
| | YCQQSKEVPW TFGQGTKVEI KRTVAAPSVE |
| | IFPPSDEQLK SGTASVVCLL NNFYPREAKV |
| | QWKVDNALQS GNSQESVTEQ DSKDSTYSLS |
| | STLTLSKADY EKHKVYACEV THQGLSSPVT |
| | KSENRGEC |
| | (SEQ ID NO: 1) |
| Heavy chain | QVQLVQSGAE VKKPGSSVKV SCKASGYTFT |
| | DYNMHWVRQA PGQGLEWIGY IYPYNGGTGY |
| | NQKFKSKATI TADESTNTAY MELSSLRSED |
| | TAVYYCARGR PAMDYWGQGT LVTVSSASTK |
| | GPSVFPLAPS SKSTSGGTAA LGCLVKDYFP |
| | EPVTVSWNSG ALTSGVHTFP AVLQSSGLYS |
| | LSSVVTVPSS SLGTQTYICN VNHKPSNTKV |
| | DKKVEPKSCD KTHTCPPCPA PELLGGPSVF |
| | LFPPKPKDTL MISRTPEVTC VVVDVSHEDP |
| | EVKENWYVDG VEVHNAKTKP REEQYNSTYR |
| | VVSVLTVLHQ DWLNGKEYKC KVSNKALPAP |
| | IEKTISKAKG QPREPQVYTL PPSRDELTKN |
| | QVSLTCLVKG FYPSDIAVEW ESNGQPENNY |
| | KTTPPVLDSD GSFFLYSKLT VDKSRWQQGN |
| | VFSCSVMHEA LHNHYTQKSL SLSPGK |
| | (SEQ ID NO: 2) |

The term “chemotherapeutic agent” means a drug (medicament or pharmaceutically active ingredient) for treating cancer.

The term “diluent” as used herein refers to a solution suitable for altering or achieving an exemplary or appropriate concentration or concentrations as described herein.

The term “administration route” refers to methods that may be used to enable delivery of the ADCs and chemotherapeutic agent to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. For administration of LNAuM for the treatment of cancer, administration into the systemic circulation by intravenous or subcutaneous administration may be desired. For treatment of a cancer characterized by a solid tumor, administration can also be localized directly into the tumor, if so desired.

The term “treatment” refers to the administration of a therapeutic agent to a subject, who has a disease with the purpose to cure, heal, alleviate, delay, relieve, alter, remedy, ameliorate, improve or affect the disease.

A “subject” is a mammal, preferably a human.

“Effective amount” means that amount of LNAuM or chemotherapeutic agent that elicits the desired biological response in a subject. Such response includes alleviation of the symptoms of the disease or disorder being treated, inhibition or a delay in the recurrence of symptom of the disease or of the disease itself, an increase in the longevity of the subject compared with the absence of the treatment, or inhibition or delay in the progression of symptom of the disease or of the disease itself. Toxicity and therapeutic efficacy of the LNAuM or chemotherapeutic agent can be determined by standard pharmaceutical procedures in cell cultures and in experimental animals. The effective amount of the LNAuM or chemotherapeutic agent to be administered to a subject will depend on the stage, category and status of the multiple myeloma and characteristics of the subject, such as general health, age, sex, body weight and drug tolerance. The effective amount of the LNAuM or chemotherapeutic agent to be administered will also depend on administration route and dosage form. Dosage amount and interval can be adjusted individually to provide plasma levels of the active compound that are sufficient to maintain desired therapeutic effects.

By “FLT3”, “FLT3 polypeptide,” or “FLT-3 Receptor,” is meant a polypeptide or fragment thereof having at least about 85%, 90%, 95%, 99% or 100% amino acid sequence identity to the human sequence of FLT3 tyrosine kinase receptor, also referred to as FLK-2 and STK-1, provided at NCBI Accession No. NP_004110 and having tyrosine kinase activity, including receptor tyrosine kinase activity.

Human FLT3 polypeptide is presented in Table 2.

TABLE 2
Human FLT3 polypeptide.

MPALARDGGQLPLLVVFSAMIFGTITNQDLPVIKCVLINH
KNNDSSVGKSSSYPMVSESPEDLGCALRPQSSGTVYEAAA
VEVDVSASITLQVLVDAPGNISCLWVFKHSSLNCQPHFDL
QNRGVVSMVILKMTETQAGEYLLFIQSEATNYTILFTVSI
RNTLLYTLRRPYERKMENQDALVCISESVPEPIVEWVLCD
SQGESCKEESPAVVKKEEKVLHELFGTDIRCCARNELGRE
CTRLFTIDLNQTPQTTLPQLFLKVGEPLWIRCKAVHVNHG
FGLTWELENKALEEGNYFEMSTYSTNRTMIRILFAFVSSV
ARNDTGYYTCSSSKHPSQSALVTIVEKGFINATNSSEDYE
IDQYEEFCFSVRFKAYPQIRCTWTFSRKSFPCEQKGLDNG
YSISKFCNHKHQPGEYIFHAENDDAQFTKMFTLNIRRKPQ
VLAEASASQASCFSDGYPLPSWTWKKCSDKSPNCTEEITE
GVWNRKANRKVFGQWVSSSTLNMSEAIKGFLVKCCAYNSL
GTSCETILLNSPGPFPFIQDNISFYATIGVCLLFIVVLTL
LICHKYKKQFRYESQLQMVQVTGSSDNEYFYVDFREYEYD
LKWEFPRENLEFGKVLGSGAFGKVMNATAYGISKTGVSIQ
VAVKMLKEKADSSEREALMSELKMMTQLGSHENIVNLLGA
CTLSGPIYLIFEYCCYGDLLNYLRSKREKFHRTWTEIFKE
HNESFYPTFQSHPNSSMPGSREVQIHPDSDQISGLHGNSF
HSEDEIEYENQKRLEEEEDLNVLTFEDLLCFAYQVAKGME
FLEFKSCVHRDLAARNVLVTHGKVVKICDFGLARDIMSDS
NYVVRGNARLPVKWMAPESLFEGIYTIKSDVWSYGILLWE
IFSLGVNPYPGIPVDANFYKLIQNGFKMDQPFYATEEIYI
IMQSCWAFDSRKRPSFPNLTSFLGCQLADAEEAMYQNVDG
RVSECPHTYQNRRPFSREMDLGLLSPQAQVEDS
(SEQ ID NO: 3)

By “FLT3-ITD” is meant a FLT3 polypeptide having internal tandem duplication(s) including but not limited to simple tandem duplication(s) and/or tandem duplication(s) with insertion. In various embodiments, FLT3 polypeptides having internal tandem duplications are activated FLT3 variants (e.g., constitutively autophosphorylated). In some embodiments, the FLT3-ITD includes tandem duplications and/or tandem duplication(s) with insertion in any exon or intron including, for example, exon 11, exon 11 to intron 11, and exon 12, exon 14, exon 14 to intron 14, and exon 15. The internal tandem duplication mutation (FLT3-ITD) is the most common FLT3 mutation, present in about 20-25% of AML cases. Subjects with FLT3-ITD AML have a worse prognosis than those with wild-type (WT) FLT3, with an increased rate of relapse and a shorter duration of response to chemotherapy.

Actual dosage levels of the LNAuM in a pharmaceutical composition of the present invention may be varied so as to obtain an amount of the LNAuM that is effective to achieve a desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well-known in the medical arts.

By “P-glycoprotein” is meant a polypeptide or fragment thereof having at least about 85% amino acid sequence identity to the human sequence provided at UniProtKB/Swiss-Prot: P08183 and conferring multi-drug resistance on a cell in which it is expressed.

The term “pharmaceutical composition” refers to a preparation which is in such form as to permit the biological activity of the active ingredient such as LNAuM to be effective (when administered to a subject), and which contains no additional components which are unacceptably toxic to a subject to which the composition would be administered. Such compositions are sterile.

Compositions or methods “comprising” one or more recited elements may include other elements not specifically recited.

Reference to a numerical range herein (e.g., “X to Y” or “from X to Y”) includes the endpoints defining the range and all values falling within the range.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.”

The anti-CD33 antibody disclosed herein is lintuzumab (HuM195) produced and characterized in Co et al. 1992 “Chimeric and humanized antibodies with specificity for the CD33 antigen” J Immunol. 148 (4): 1149-54 and Caron et al. 1992 “Biological and Immunological Features of Humanized M195 (Anti-CD33) Monoclonal Antibodies” CANCER RESEARCH 52, 6761-6767 which are herein incorporated by reference for all purposes.

The light and heavy chains of lintuzumab are provided as SEQ ID NO:1 and SEQ ID NO: 2, respectively (Table 1).

LNAuM is an ADC wherein the drug component is a MMAU (monomethylauristatin E β-D-glucuronide).

MMAU is of the structure:

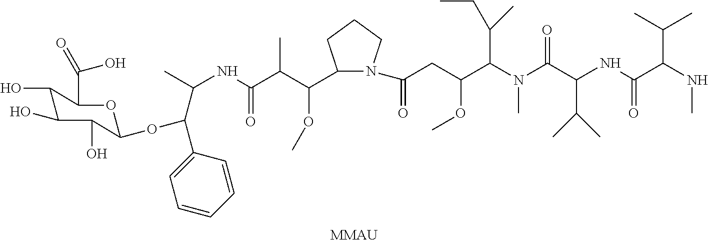

MMAU is a derivative of Monomethyl Auristatin E (MMAE).

The biological activity of MMAU is thought to involve release of glucuronic acid in lysosome resulting formation of MMAE and followed by MMAE binding to tubulin and inhibition of tubulin polymerization and cell division.

LNAuM comprises a linker between the MMAU and the lintuzumab. The linker comprises a cleavable unit-β-Ala-Val-Ser (Glc)-. The linker further comprises a maleimide group for linkage to the antibody. The linker further comprises a self-immolative group p-aminobenzyl alcohol (PAB) unit.

LNAuM is of Formula (I):

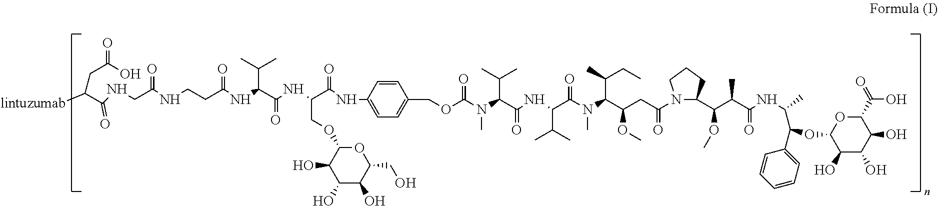

wherein n represents the number of drug-linker molecules per antibody.

The variable n ranges from 1 to 20 and is preferably from 1 to 8.

In an embodiment, n is 1.

In an embodiment, n is 2.

In an embodiment, n is 3.

In an embodiment, n is 4.

In an embodiment, n is 5.

In an embodiment, n is 6.

In an embodiment, n is 7.

In an embodiment, n is 8.

Depending on the context, n can represent the average number of drug-linker molecules per antibody, also referred to the drug-antibody ratio (DAR).

For example, a pharmaceutical composition comprising LNAuM of Formula (I) may have a DAR of ≥1, or in the range of 1 to 20, or 1 to 15, or 1 to 10, or 2 to 10, or 2 to 9, or 2 to 8, or 3 to 8, or 4 to 8, or 5 to 8, or 6 to 8, or 7 to 8.

In an embodiment, a pharmaceutical composition comprising LNAuM of Formula (I) has a drug-to-antibody ratio in the range of 1 to 8, or 2 to 8, or 3 to 8, or 4 to 8, or 5 to 8, or 6 to 8, or 7 to 8, or 7.5 to 8.

In an embodiment, a pharmaceutical composition comprising LNAuM of Formula (I) has a drug-to-antibody ratio 7.

In an embodiment, a pharmaceutical composition comprising LNAuM of Formula (I) has a drug-to-antibody ratio 7.1.

In an embodiment, a pharmaceutical composition comprising LNAuM of Formula (I) has a drug-to-antibody ratio 7.2.

In an embodiment, a pharmaceutical composition comprising LNAuM of Formula (I) has a drug-to-antibody ratio 7.3.

In an embodiment, a pharmaceutical composition comprising LNAuM of Formula (I) has a drug-to-antibody ratio 7.4.

In an embodiment, a pharmaceutical composition comprising LNAuM of Formula (I) has a drug-to-antibody ratio 7.5.

In an embodiment, a pharmaceutical composition comprising LNAuM of Formula (I) has a drug-to-antibody ratio 7.6.

In an embodiment, a pharmaceutical composition comprising LNAuM of Formula (I) has a drug-to-antibody ratio 7.7.

In an embodiment, a pharmaceutical composition comprising LNAuM of Formula (I) has a drug-to-antibody ratio 7.8.

In an embodiment, a pharmaceutical composition comprising LNAuM of Formula (I) has a drug-to-antibody ratio 7.9.

In an embodiment, a pharmaceutical composition comprising LNAuM of Formula (I) has a drug-to-antibody ratio 8.0.

In an embodiment, n, or drug-to-antibody (DAR) ratio of the LNAuM may be determined using a ESI-MS or reverse phase high pressure liquid chromatography.

As a skilled person will understand, a composition or a pharmaceutical composition comprising LNAuM of Formula (I) may comprise a mixture of different LNAuM molecules in which n is different. For example, when DAR for a pharmaceutical composition comprising LNAuM of Formula (I) is 7.8, the composition may predominantly comprise LNAuM molecules in which n is 8, as well as minor amounts of LNAuM molecules in which n is smaller than 8, for example 7 and 6, and possibly trace amounts of molecules in which n is smaller than 6. n, or DAR, is therefore not necessarily an integer. If the (theoretical) maximum number of MMAU molecules to be conjugated to the LNAuM molecule is 8, then the DAR should in principle not exceed 8 or about 8, but the composition may comprise minor amounts of LNAuM molecules in which n is larger than 8, for example 9. The DAR may depend on e.g. the number of possible conjugation sites in the antibody and the number of payload molecules that may be conjugated to a single conjugation site, and/or the extent to which the possible conjugation sites in the LNAuM are in fact conjugated to a payload molecule.

As used herein a “LNAuM” refers to the ADC of Formula (I). The antibody portion comprises the light chain of SEQ ID NO:1 and the heavy chain of SEQ ID NO:2 (Table 1).

Methods to make the linker-MMAUs and/or LNAuM are disclosed in PCT publications WO/2016/001485, WO/2018/234636, and WO/2022/175595, all of which are incorporated by reference for all purposes.

The present invention provides methods for treating subjects with cancer, in particular a hematologic cancer, such as AML, by administering LNAuM and chemotherapeutic agent.

As used herein, a “hematologic cancer” is a cancer that begins in blood-forming tissue, such as the bone marrow, or in the cells of the immune system. Examples of hematologic cancer are leukemia, lymphoma and multiple myeloma.

CD33 expressing hematologic cancers which can be treated using the disclosed methods include acute myeloid leukemia (AML), chronic myeloid leukemia (CML), acute lymphoblastic leukemia (ALL), myelodysplastic syndrome (MDS), and acute promyelocytic leukaemia (APL). The cancer can be chemotherapy sensitive; alternatively, the cancer can be chemotherapy resistant.

In yet another embodiment, the acute myeloid leukemia is refractory or relapsed acute myeloid leukemia.

The invention also provides methods of treating a hematologic cancer having at least one negative prognostic factor, e.g., FLT3 internal tandem duplication.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The compositions depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, antibodies can be in lyophilized form for constitution with a suitable vehicle, e.g., sterile pyrogen-free water, before use.

The LNAuM of Formula (I) or the pharmaceutical composition comprising LNAuM of Formula (I) may be particularly useful in combination with chemotherapeutic agents. Thus, the present disclosure provides a combination of LNAuM of Formula (I), or its pharmaceutical composition, in a combination with chemotherapeutic agent for simultaneous, separate or sequential administration. The LNAuM of Formula (I) and a chemotherapeutic agent can act additively or synergistically. A synergistic combination of the LNAuM of Formula (I) and a chemotherapeutic agent might allow the use of lower dosages of one or both of these agents and/or less frequent dosages of one or both of the LNAuM of Formula (I) and the chemotherapeutic agent and/or to administer the chemotherapeutic agent less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of the agent.

The chemotherapeutic agent can be administered according to therapeutic protocols well known in the art. It will be apparent to those skilled in the art that the administration of the chemotherapeutic agent can be varied depending on the disease being treated and the known effects of the chemotherapeutic agent on that disease. Also, in accordance with the knowledge of the skilled clinician, the therapeutic protocols (e.g., dosage amounts and times of administration) can be varied in view of the observed effects of the administered chemotherapeutic agents on the subject, and in view of the observed responses of the disease to the agents, and observed adverse effects.

In an embodiment, the present invention provides a method of treating CD33 expressing cancer in a subject comprising administering to the subject an effective amount of chemotherapeutic agent and an effective amount of LNAuM of Formula (I).

In an embodiment, the cancer is a hematologic cancer.

In an embodiment, the hematologic cancer is leukemia.

In an embodiment, the leukemia is selected from the group consisting of acute myeloid leukemia (AML), chronic myeloid leukemia (CML), acute lymphoblastic leukemia (ALL), myelodysplastic syndrome (MDS), and acute promyelocytic leukaemia (APL).

In an embodiment, the cancer is acute myeloid leukemia (AML).

In an embodiment, the acute myeloid leukemia (AML) is refractory or relapse acute myeloid leukemia.

In an embodiment, the acute myeloid leukemia (AML) is characterized by FLT3 internal tandem duplication (FLT3-ITD).

In an embodiment, the chemotherapeutic agent is selected from the group consisting of an FLT3 inhibitor and a BCL2 inhibitor.

In an embodiment, the FLT3 inhibitor is selected from the group consisting of midostaurin and gilteritinib fumarate.

In an embodiment, the BCL2 inhibitor is venetoclax.

In an embodiment, the chemotherapeutic agent is selected from the group consisting cytarabine and daunorubicin-HCl.

In an embodiment, the chemotherapeutic agent is venetoclax.

In an embodiment, venetoclax is administered at a daily dose of at least 10 mg, at least 20 mg, at least 50 mg, at least 100 mg, at least 200 mg or at least 400 mg.

In an embodiment, the chemotherapeutic agent is cytarabine.

In an embodiment, cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

In an embodiment, the chemotherapeutic agent is midostaurin.

In an embodiment, midostaurin is administered daily at least a dose of 30 mg/m², 45 mg/m² or 60 mg/m².

In an embodiment, the chemotherapeutic agent is gilteritinib.

In an embodiment, gilteritinib is administered at a dose at least about 5 mg/day, at least about 10 mg/day, at least about 40 mg/day, or at least about 80 mg/day, or at least about 120 mg/day.

In an embodiment, the chemotherapeutic agent is daunorubicin.

In an embodiment, daunorubicin is administered at a dose at least about 10 mg/m²/day, at least about 30 mg/m²/day, at least about 60 mg/m²/day, or at least about 90 mg/m²/day.

In an embodiment, LNAuM is administered at a dose of at least about 0.1 mg/kg, at least about 0.3 mg/kg, at least about 0.5 mg/kg, at least about 0.8 mg/kg, at least about 1 mg/kg, at least about 1.5 mg/kg, at least about 2 mg/kg, at least about 2.5 mg/kg, at least about 3 mg/kg, at least about 3.5 mg/kg, or at least about 4 mg/kg.

In an embodiment, the invention provides a method of treating CD33 expressing acute myeloid leukemia (AML) in a subject comprising a step of administering cytarabine and LNAuM of Formula (I),

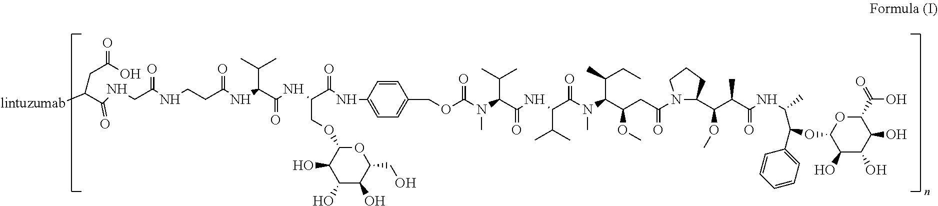

wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

In an embodiment, the invention provides a method of treating CD33 expressing acute myeloid leukemia (AML) in a subject comprising the step of administering venetoclax and LNAuM of Formula (I),

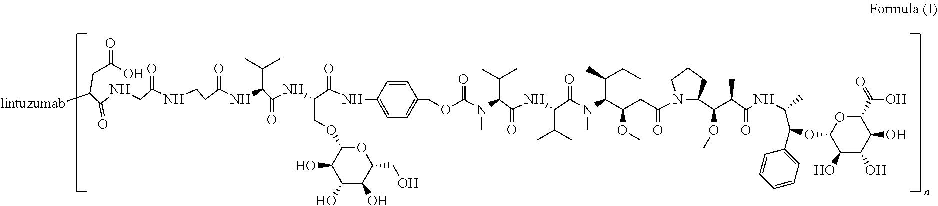

wherein venetoclax is administered at a daily dose of least 10 mg, at least 20 mg, at least 50 mg, at least 100 mg, at least 200 mg or at least 400 mg.

In an embodiment, the invention provides a method of treating CD33 expressing acute myeloid leukemia (AML) in a subject comprising the step of administering midostaurin and LNAuM of Formula (I),

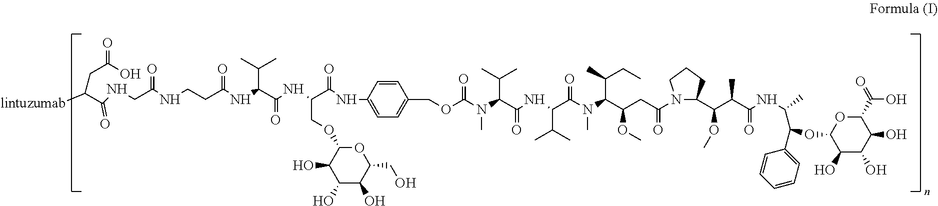

wherein midostaurin is administered daily at least a dose of 30 mg/m², 45 mg/m² or 60 mg/m².

In an embodiment, the invention provides a method of treating CD33 expressing acute myeloid leukemia (AML) in a subject comprising the step of administering gilteritinib and LNAuM of Formula (I),

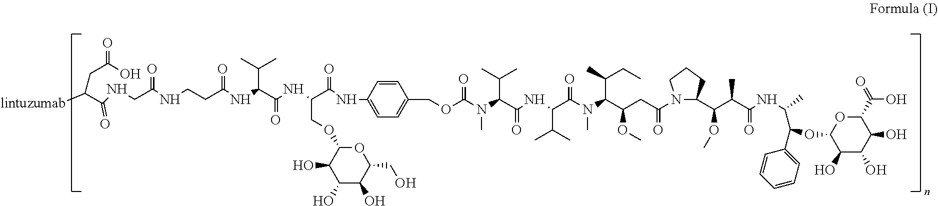

wherein gilteritinib is administered at a dose at least about 5 mg/day, at least about 10 mg/day, at least about 40 mg/day, or at least about 80 mg/day, or at least about 120 mg/day.

In an embodiment, the invention provides a method of treating CD33 expressing acute myeloid leukemia (AML) in a subject comprising a step of administering daunorubicin and LNAuM of Formula (I),

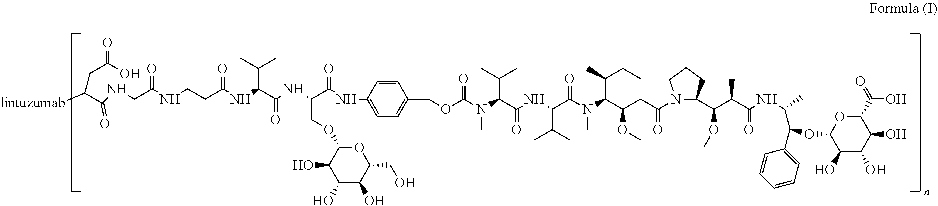

wherein daunorubicin is administered at a dose at least about 10 mg/m²/day, at least about 30 mg/m²/day, at least about 60 mg/m²/day, or at least about 90 mg/m²/day.

In an embodiment, the invention provides a method of treating CD33 expressing acute myeloid leukemia (AML) in a subject comprising a step of administering cytarabine, venetoclax and LNAuM of Formula (I),

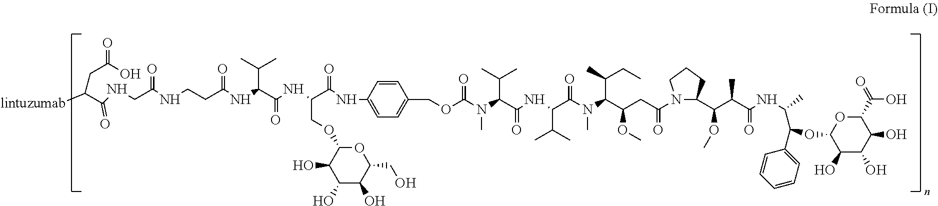

wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered venetoclax is administered at a daily dose of at least 10 mg, at least 20 mg, at least 50 mg, at least 100 mg, at least 200 mg or at least 400 mg.

In one embodiment, chemotherapeutic agent is administered to the subject prior to the administration of the LNAuM. In another embodiment, chemotherapeutic agent and the LNAuM are administered to the subject concurrently.

In one embodiment, cytarabine is administered to the subject prior to the administration of the LNAuM. In another embodiment, cytarabine and the LNAuM are administered to the subject concurrently.

In one embodiment, venetoclax is administered to the subject prior to the administration of the LNAuM. In another embodiment, venetoclax and the LNAuM are administered to the subject concurrently.

In one embodiment, midostaurin is administered to the subject prior to the administration of the LNAuM. In another embodiment, midostaurin and the LNAuM are administered to the subject concurrently.

In one embodiment, gilteritinib is administered to the subject prior to the administration of the LNAuM. In another embodiment, gilteritinib and the LNAuM are administered to the subject concurrently.

In one embodiment, daunorubicin is administered to the subject prior to the administration of the LNAuM. In another embodiment, cytarabine and the LNAuM are administered to the subject concurrently.

### EXAMPLES

In the following, the present invention will be described in more detail. Reference will now be made in detail to the embodiments, examples of which are illustrated in the accompanying drawings. The description below discloses some embodiments in such detail that a person skilled in the art is able to utilize the invention based on the disclosure. Not all steps of the embodiments are discussed in detail, as many of the steps will be obvious for the person skilled in the art based on this specification.

All chemotherapeutic agents were from MedChemExpress: Midostaurin (HY-10230/CS-3331), Cytarabine (HY-13605/CS-2177), Venetoclax (HY-15531/CS-1155), Daunorubicin-HCl (HY-13062/CS-1271), Midostaurin (HY-10230), and Gilteritinib hemifumarate (HY-12432A).

### Example 1—HL-60 Cells

HL-60 cells (ATCC®, cat. no. CCL-240™), derived from human promyelocytic leukemia, were cultured in Iscove's Modified Dulbecco's Medium (Gibco, Life Technologies) supplemented with 20% heat inactivated FBS (Gibco, Life Technologies). Cells were maintained in a humidified atmosphere containing 5% CO₂ at +37° C.

For toxicity assay, HL-60 cells were seeded for 7500 cells/well of 96-well plate. The chemotherapeutic agents were added as 2-fold stock solutions and incubated with cells for 5 days.

Midostaurin was tested at concentrations of 0.01, 0.1, 1, 10, 100, and 1000 nM. All other compounds were tested at concentrations 0.1, 1, 10, 100, 1000, and 10 000 nM. After 5 days incubation, the viability of the cells was determined with PrestoBlue Cell Viability Reagent (Life Technologies) according to the manufacturer's instructions. The percentage of viable cells was calculated by dividing the sample absorbance value with untreated cell control value and multiplying by 100%.

Testing of midostaurin and gilteritinib in HL-60 cells was performed as above but incubation time was 4 days. All compounds were tested at concentrations of 0.1-10 000 nM.

IC50 values were determined after average % and standard deviations were transferred to GraphPad Prism 9.1.2. Dose-response curves were generated and the IC50% values of the samples were obtained by non-linear regression analysis (Inhibitor vs. response, variable slope (four parameters)) using the software. The IC50 values are shown in Table 1-1.

TABLE 1-1
IC50 values of HL-60 cells per chemotherapeutic agent.

| Compound | IC50 |
|---|---|
| Cytarabine | 298 nM |
| Venetoclax | 112 nM |
| Daunorubicin-HCl | 15 nM |
| Midostaurin | 800 nM |
| Gilteritinib hemifumarate | 425 nM |

### Combination Study of LNAuM and Cytarabine

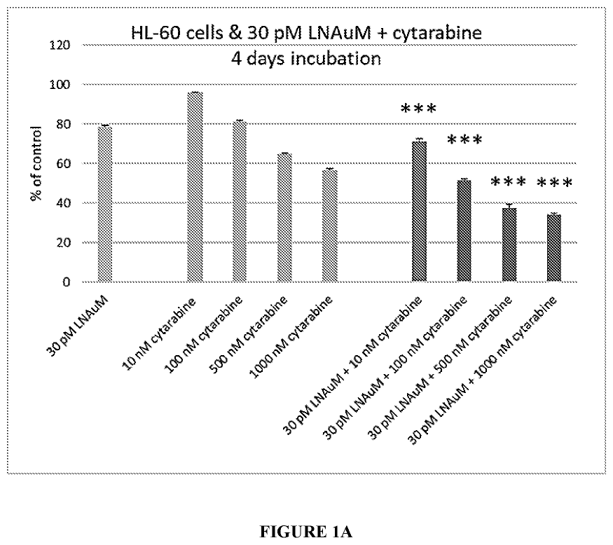

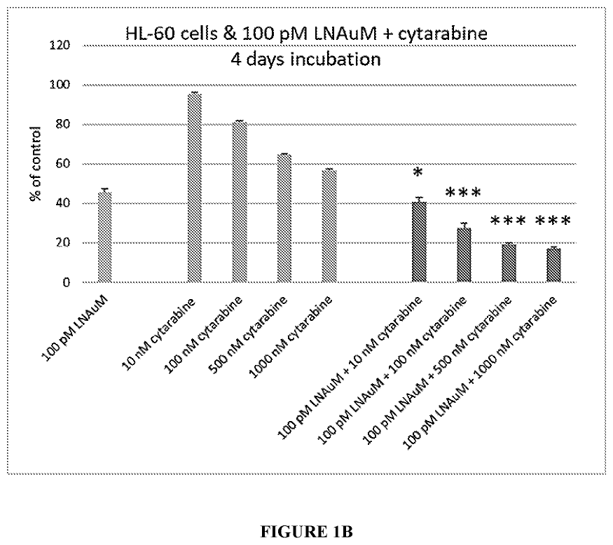

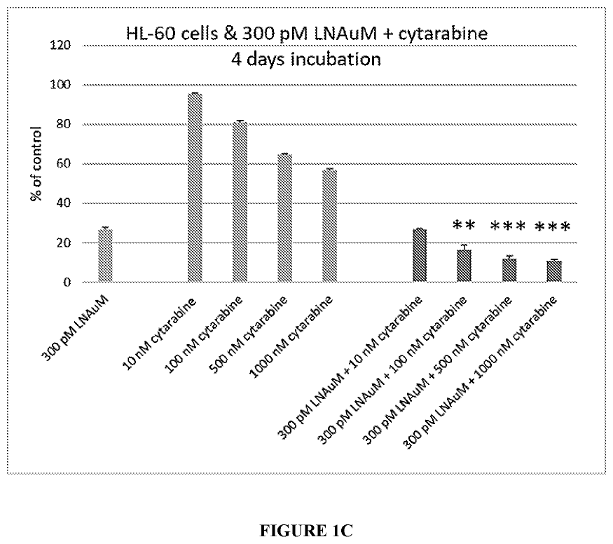

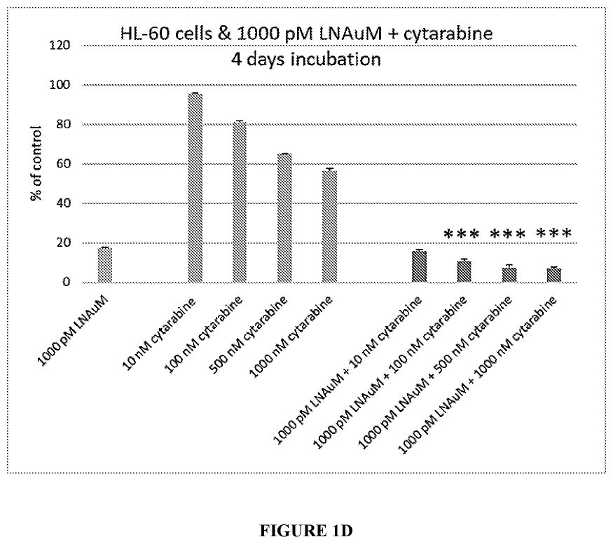

HL-60 cells were maintained as described above. Cells were seeded (7500 5 cells/well) to 96-well plate at volume of 50 μl. HL-60 cells were treated with 30, 100, 300 or 1000 pM LNAuM in the presence or absence of 10, 100, 500 or 1000 nM cytarabine. Dilutions (25 μl) of LNAuM and cytarabine were added as 4-fold concentrations. Incubation time was 4 days. The viability of the cells was determined as above. Results are shown in FIG. 1.

Results show that LNAuM+cytarabine combinations at concentrations 100 pM-1 nM of LNAuM and 500-1000 nM of cytarabine reduce HL-60 cell viability more than 50% compared to single agent cell survival (bold underlined in Table 1-2) indicating strong synergistic effect.

TABLE 1-2
n-fold change and % decrease as to combination, HL-60 cells.

| LNAuM | Cytarabine | LNAuM | | LNAuM % | Drug % |
|---|---|---|---|---|---|
| concentration | concentration | fold to | Drug fold to | decrease to | decrease to |
| (nM) | (nM) | combination | combination | combination | combination |
| 0.03 | 10 | 1.1 | 1.3 | 9.1% | 25.3% |
| 0.03 | 100 | 1.5 | 1.6 | 34.1% | 36.2% |
| 0.03 | 500 | 2.1 | 1.7 | 52.4% | 42.5% |
| 0.03 | 1000 | 2.3 | 1.6 | 56.2% | 39.4% |
| 0.1 | 10 | 1.1 | 2.3 | 10.9% | 57.4% |
| 0.1 | 100 | 1.7 | 3.0 | 40.3% | 66.4% |
| 0.1 | 500 | 2.4 | 3.4 | 57.6% | 70.2% |
| 0.1 | 1000 | 2.7 | 3.3 | 62.6% | 69.9% |
| 0.3 | 10 | 1.0 | 3.6 | 0.1% | 72.3% |
| 0.3 | 100 | 1.6 | 4.9 | 38.1% | 79.7% |
| 0.3 | 500 | 2.3 | 5.5 | 55.9% | 82.0% |
| 0.3 | 1000 | 2.5 | 5.3 | 59.7% | 81.1% |
| 1 | 10 | 1.1 | 6.1 | 8.9% | 83.6% |
| 1 | 100 | 1.6 | 7.5 | 37.1% | 86.7% |
| 1 | 500 | 2.3 | 8.7 | 56.6% | 88.5% |
| 1 | 1000 | 2.5 | 8.3 | 60.0% | 87.9% |

Combination Study of LNAuM with Venetoclax or Daunorubicin

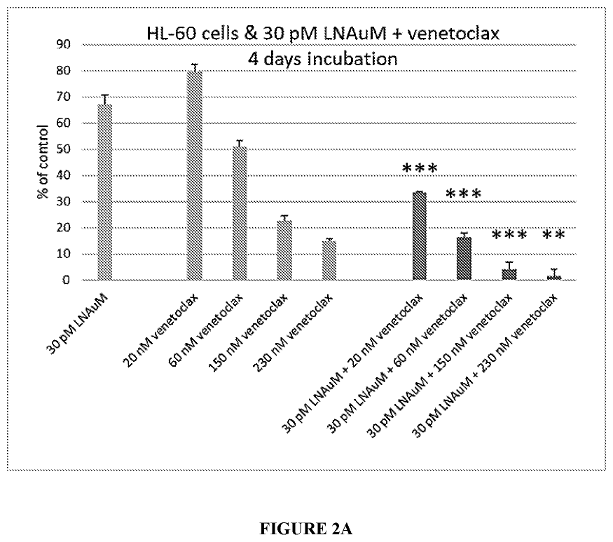

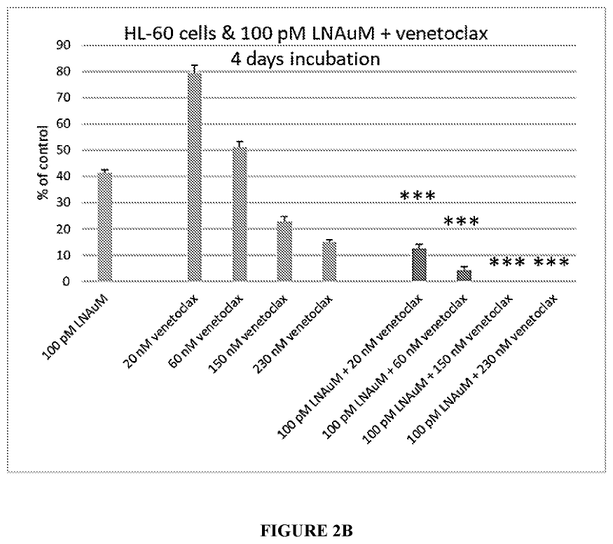

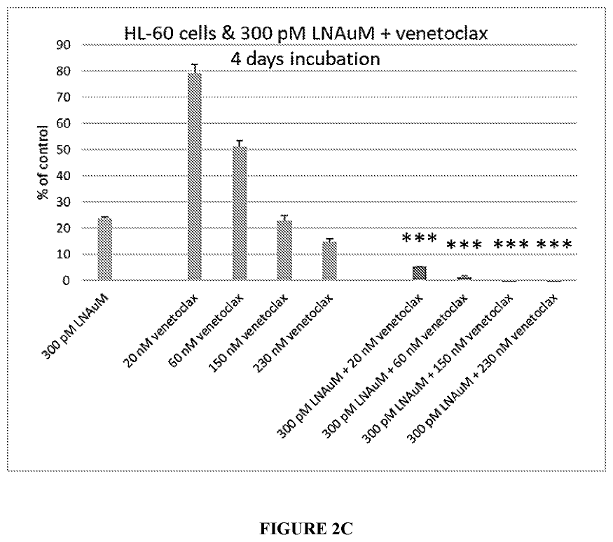

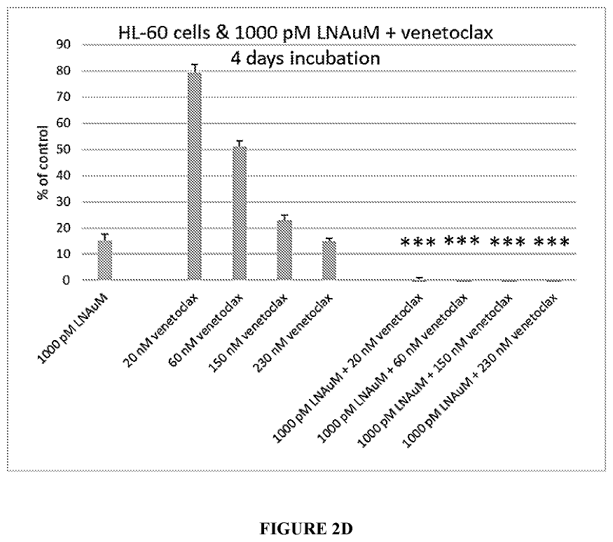

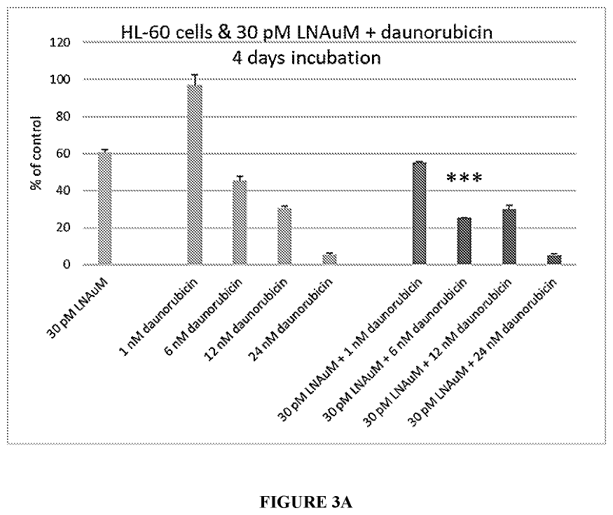

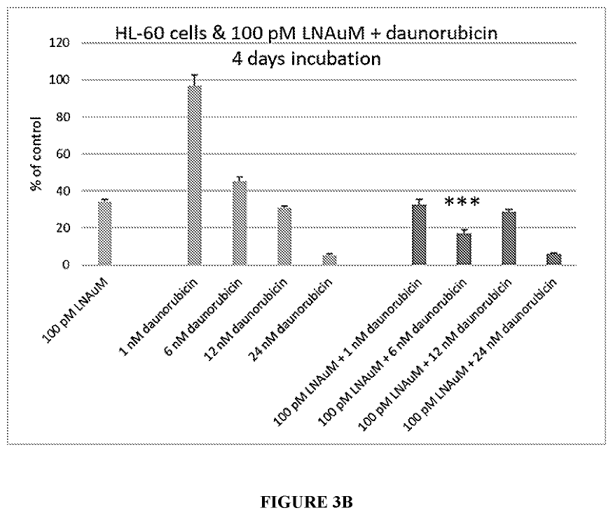

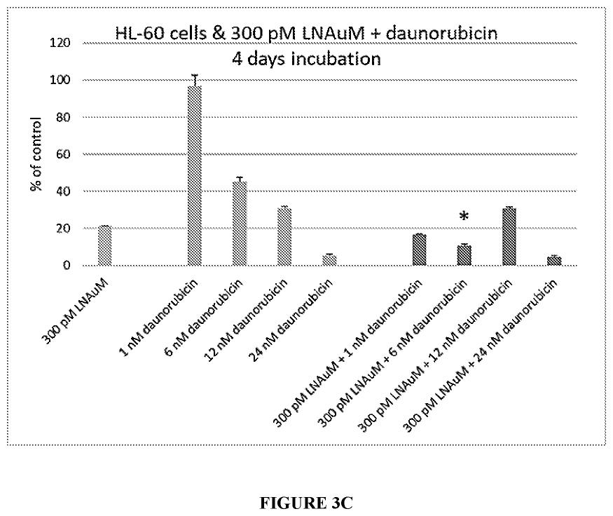

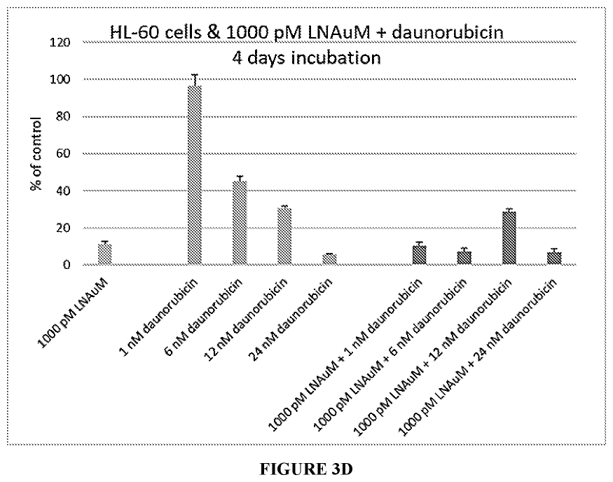

HL-60 cells were maintained as above. Cells were seeded (7500 cells/well) to 96-well plate at volume of 50 μl. HL-60 cells were treated with 30, 100, 300 or 1000 pM LNAuM in the presence or absence venetoclax or daunorubicin. Venetoclax was tested at concentrations of 20, 60, 150, or 230 nM. Daunorubicin was tested at concentrations of 1, 6, 12, or 24 nM. LNAuM, venetoclax and daunorubicin were added as 4-fold concentrations (volume 25 μl). Incubation time was 4 days. The viability of the cells was determined as above. Results are shown in FIGS. 2 and 3.

Results show that LNAuM+venetoclax combinations at all concentration pairings reduce HL-60 cell viability more than 50% compared to single agent cell survival indicating very strong synergistic effect (Table 1-3).

TABLE 1-3
n-fold change and % decrease as to combination, HL-60 cells.

| LNAuM | Venetoclax | LNAuM | | LNAuM % | Drug % |
|---|---|---|---|---|---|
| concentration | concentration | fold to | Drug fold to | decrease to | decrease to |
| (nM) | (nM) | combination | combination | combination | combination |
| 0.03 | 20 | 2.0 | 2.4 | 50.1% | 57.7% |
| 0.03 | 60 | 4.1 | 3.1 | 75.8% | 68.2% |
| 0.03 | 150 | 16.5 | 5.6 | 94.0% | 82.2% |
| 0.03 | 230 | 38.8 | 8.6 | 97.4% | 88.3% |
| 0.1 | 20 | 3.4 | 6.4 | 70.2% | 84.4% |
| 0.1 | 60 | 10.2 | 12.6 | 90.2% | 92.1% |
| 0.1 | 150 | #DIV/0! | #DIV/0! | 100.0% | 100.0% |
| 0.1 | 230 | #DIV/0! | #DIV/0! | 100.0% | 100.0% |
| 0.3 | 20 | 4.5 | 15.1 | 77.9% | 93.4% |
| 0.3 | 60 | 19.6 | 42.0 | 94.9% | 97.6% |
| 0.3 | 150 | #DIV/0! | #DIV/0! | 100.0% | 100.0% |
| 0.3 | 230 | #DIV/0! | #DIV/0! | 100.0% | 100.0% |
| 1 | 20 | #DIV/0! | #DIV/0! | 100.0% | 100.0% |
| 1 | 60 | #DIV/0! | #DIV/0! | 100.0% | 100.0% |
| 1 | 150 | #DIV/0! | #DIV/0! | 100.0% | 100.0% |
| 1 | 230 | #DIV/0! | #DIV/0! | 100.0% | 100.0% |

Table 1-4 and FIG. 3 show that LNAuM+daunorubicin combinations at some concentration pairings reduce HL-60 cell viability almost 50% compared to single agent cell survival (bold underlined in Table 1-4) indicating at least additive effect.

TABLE 1-4
n-fold change and % decrease as to combination. Daunorubicin results
of 12 nM have been stroked through due to erroneous data.

| LNAuM | Daunorubicin | LNAuM | | LNAuM % | Drug % |
|---|---|---|---|---|---|
| concentration | concentration | fold to | Drug fold to | decrease to | decrease to |
| (nM) | (nM) | combination | combination | combination | combination |
| 0.03 | 1 | 1.1 | 1.8 | 9.2% | 43.1% |
| 0.03 | 6 | 2.4 | 1.8 | 58.0% | 43.8% |
| [symbol] | [symbol] | [symbol] | [symbol] | [symbol] | [symbol] |
| 0.03 | 24 | 12.0 | 1.1 | 91.7% | 7.4% |
| 0.1 | 1 | 1.0 | 3.0 | 4.3% | 66.2% |
| 0.1 | 6 | 2.0 | 2.6 | 49.6% | 61.9% |
| [symbol] | [symbol] | [symbol] | [symbol] | [symbol] | [symbol] |
| 0.1 | 24 | 5.6 | 0.9 | 82.2% | −11.2% |
| 0.3 | 1 | 1.3 | 5.8 | 22.0% | 82.9% |
| 0.3 | 6 | 2.0 | 4.2 | 49.5% | 76.3% |
| [symbol] | [symbol] | [symbol] | [symbol] | [symbol] | [symbol] |
| 0.3 | 24 | 4.6 | 1.2 | 78.0% | 14.6% |
| 1 | 1 | 1.1 | 9.4 | 8.0% | 89.3% |
| 1 | 6 | 1.6 | 6.4 | 36.8% | 84.3% |
| [symbol] | [symbol] | [symbol] | [symbol] | [symbol] | [symbol] |
| 1 | 24 | 1.6 | 0.8 | 38.6% | −26.3% |

### Synergy Score Calculations

Excel spread sheet containing viability data were uploaded into SynergyFinder 3.0. (https://synergyfinder.fimm.fi; Ianevski et al. 2022 SynergyFinder 3.0: an interactive analysis and consensus interpretation of multi-drug synergies across multiple samples. Nucleic Acids Research. gkac382, https://doi.org/10.1093/nar/gkac382) and synergy scores were calculated using following parameters: Readout: viability; Detect outliers: Yes; Curve Fitting: LL4; Synergy score method: ZIP; Correction: Yes.

The Zero interaction potency (ZIP) model captures the drug interaction relationships by comparing the change in the potency (effect at certain dose level) of the dose-response curves between individual drugs and their combinations. ZIP assumes that two non-interacting drugs are expected to incur minimal changes in their dose-response curves. The formulation of the model can be found at Yadav et al. 2015. Searching for Drug Synergy in Complex Dose-Response Landscapes Using an Interaction Potency Model. Comput Struct Biotechnol J. 13:504-13.

The ZIP model takes the advantages of both the Loewe additivity and the Bliss independence models, aiming at a systematic assessment of various types of drug interactions patterns that may arise in a high-throughput drug combination screening. Synergy scores can be interpreted as the average excess response due to drug interactions (i.e. synergy score of 15 corresponds to 15% of response beyond expectation).

ZIP synergy score can be interpreted as follows: If score is less than-10, interaction is antagonistic; if score is between-10 and +10, interaction is additive; and if score is more than 10, interaction is synergistic.

The ZIP synergy scores are shown in Table 1-5.

When calculating daunorubicin synergies score, 12 nM daunorubicin values were omitted.

TABLE 1-5
Summary of ZIP synergy scores per combination.
Venetoclax and cytarabine show synergistic
effect while daunorubicin effect is additive.

| Assay | ZIP Score |
|---|---|
| LNAuM + cytarabine | 11.5 (most synergistic area score 15.5) |
| LNAuM + venetoclax | 21.4 (most synergistic area score 26.2) |
| LNAuM + daunorubicin | 1.5 (most synergistic area score 2.6) |

### Example 2—KG-1 Cells

Toxicity of chemotherapeutic agents was tested with high CD33-positive KG-1 cell line (DSMZ-German Collection of Microorganisms and Cell Cultures; Cat. No. ACC 14). KG-1 cells were seeded to non-treated 96-well plate 15000 cells/well and diluted compounds were added to cells in duplicates. Dilution series (1:10) of compounds were prepared starting from 20 μM to 0.2 nM and when added to cells they diluted 2× more. Cell control was untreated cells in culture medium.

Cells with chemotherapeutic agents were cultured for 4 days and then the viability was measured with PrestoBlue cell viability reagent according to the manufacturer's (Life Technologies) instructions. Results were calculated from 5-hour Prestoblue reaction and the percentage of viable cells was calculated by dividing the sample absorbance value with cell control value.

IC50 values were determined as described in Example 1. The IC50 values are shown in Table 2-1.

TABLE 2-1
IC50 values of KG-1 cells per chemotherapeutic agent.

| Compound | IC50 | |
|---|---|---|
| Cytarabine | 230 | nM |
| Venetoclax | 2.3 | μM |
| Daunorubicin-HCl | 55 | nM |
| Midostaurin | 517 | nM |
| Gilteritinib hemifumarate | 1.7 | μM |

Combination Study of LNAuM with Venetoclax

KG-1 cells were cultured as above. Cells were seeded 15 000 cells/well to 96-well plate at volume of 50 μl. KG-1 cells were treated with 10 pM, 200 pM, 700 pM, or 4 nM of LNAuM in the presence or absence of venetoclax. Venetoclax was tested at concentrations 1, 200, 800 and 2000 nM. Dilutions (a 25 μl) of LNAuM or venetoclax were added as 4-fold concentrations to cells. Each 96-well plate had its' own small molecule, cell and medium controls. Incubation time was 4 days.

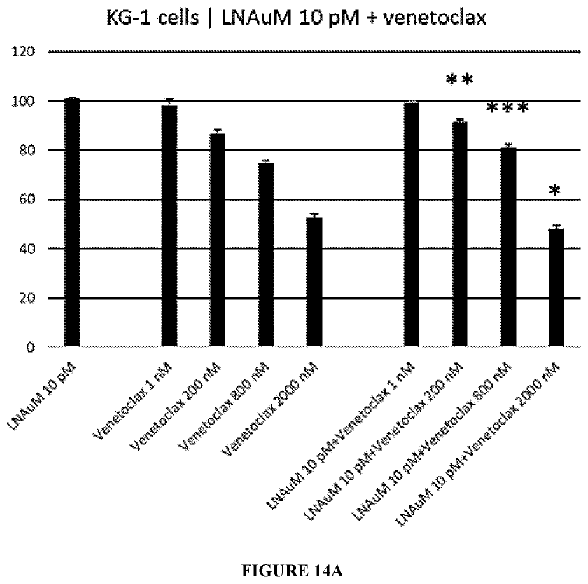

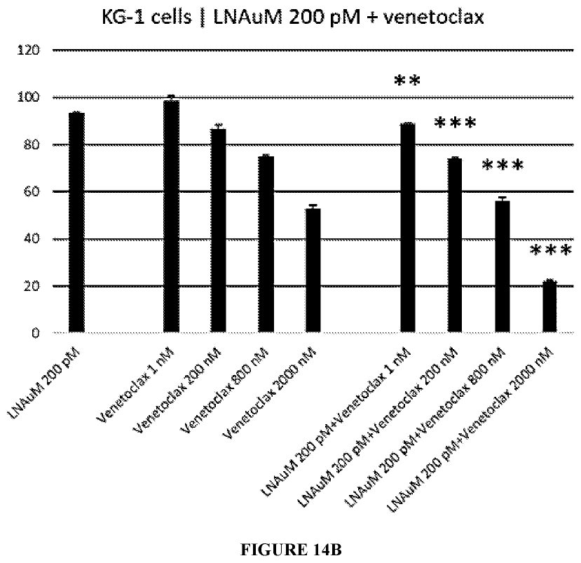

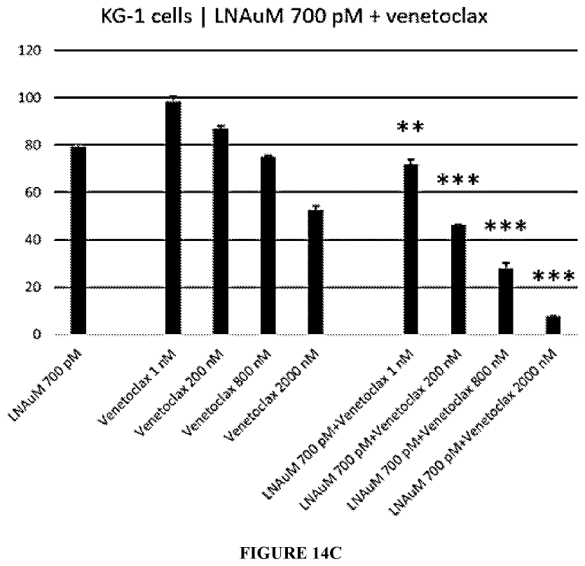

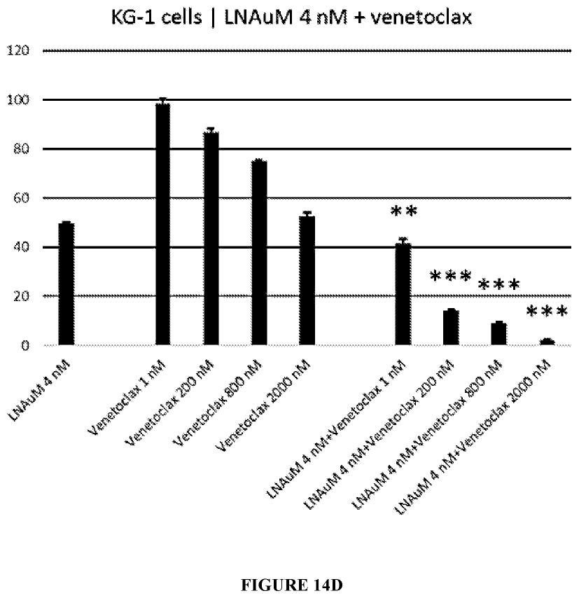

The viability of the cells was determined with PrestoBlue Cell Viability Reagent (Life Technologies) according to the manufacturer's instructions. The percentage of viable cells was calculated by dividing the sample absorbance value with untreated cell control value and multiplying by 100. The viability percentages for venetoclax alone, without LNAuM, were calculated as averages of two 96-well plates. For statistical analysis and synergy score calculations the cell control values were calculated as averages of the two 96-well plates. Results are shown on FIG. 14.

TABLE 2-2
n-fold change and % decrease as to combination, KG-1
cells. Bold indicate more than 40% reduction.

| | | | | LNAuM % | Drug % |
|---|---|---|---|---|---|
| LNAuM | Venetoclax | LNAuM | Drug | decrease | decrease |
| concen- | concen- | fold to | fold to | to | to |
| tration | tration | combi- | combi- | combi- | combi- |
| (nM) | (nM) | nation | nation | nation | nation |
| 0.01 | 1 | 1.0 | 1.0 | 1.4% | −1.0% |
| 0.01 | 200 | 1.1 | 0.9 | 9.4% | −5.4% |
| 0.01 | 800 | 1.2 | 0.9 | 19.7% | −8.2% |
| 0.01 | 2000 | 2.1 | 1.1 | 52.5% | 8.9% |
| 0.2 | 1 | 1.1 | 1.1 | 4.9% | 9.8% |
| 0.2 | 200 | 1.3 | 1.2 | 20.8% | 14.7% |
| 0.2 | 800 | 1.7 | 1.3 | 40.1% | 25.4% |
| 0.2 | 2000 | 4.3 | 2.4 | 76.6% | 58.3% |
| 0.7 | 1 | 1.1 | 1.4 | 9.3% | 27.0% |
| 0.7 | 200 | 1.7 | 1.9 | 41.8% | 46.8% |
| 0.7 | 800 | 2.8 | 2.7 | 64.6% | 62.5% |
| 0.7 | 2000 | 10.1 | 6.7 | 90.1% | 85.1% |
| 4 | 1 | 1.2 | 2.4 | 16.3% | 57.7% |
| 4 | 200 | 3.5 | 6.1 | 71.6% | 83.7% |
| 4 | 800 | 5.4 | 8.1 | 81.4% | 87.6% |
| 4 | 2000 | 21.9 | 23.1 | 95.4% | 95.7% |

Table 2-2 and FIG. 14 show that LNAuM+venetoclax combinations at concentrations 700 pM/4 nM+200/800/2000 nM pairings reduce KG-1 cell viability more than 40% compared to single agent cell survival indicating very strong synergistic effect.

Combination Study of LNAuM with Cytarabine

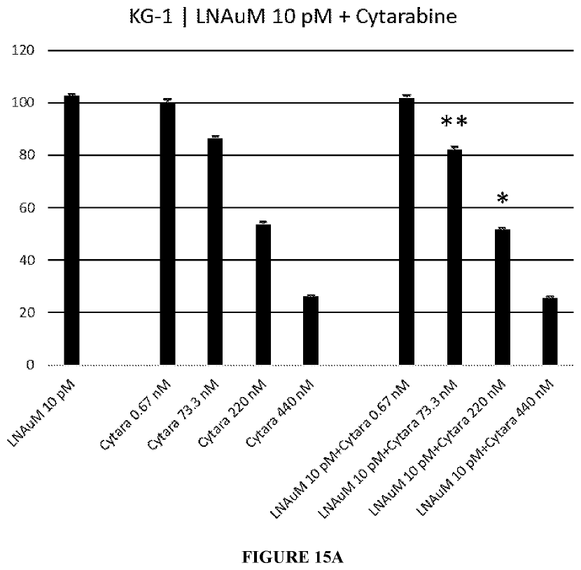

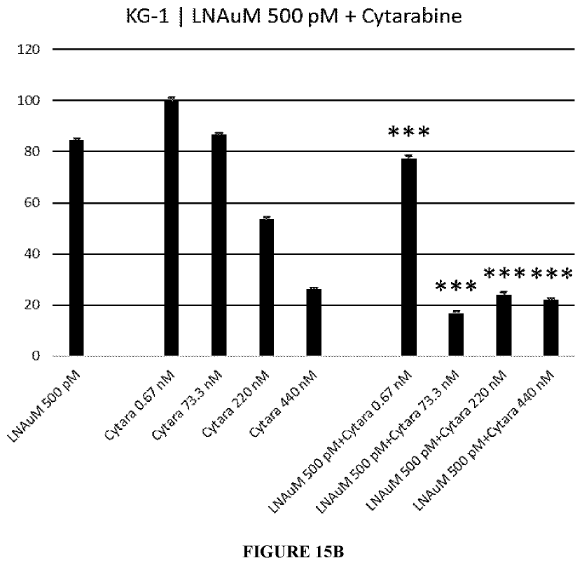

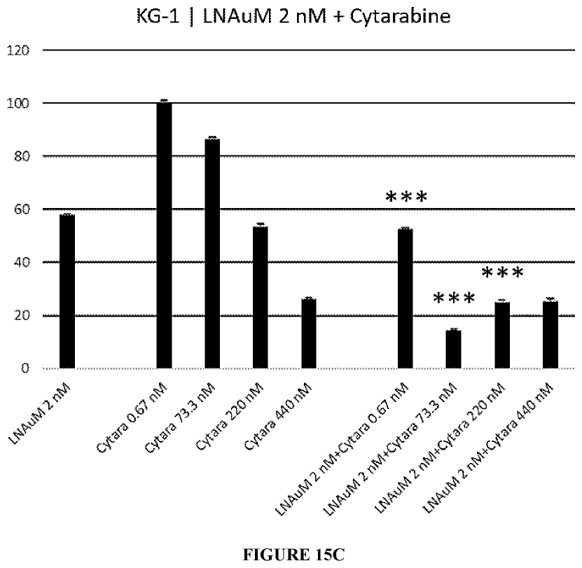

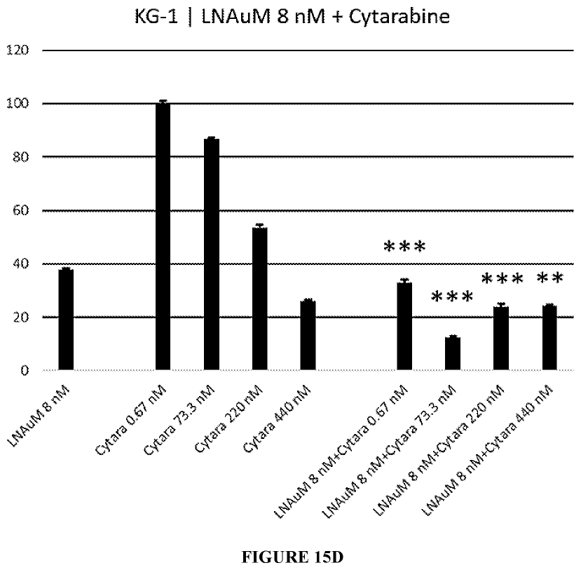

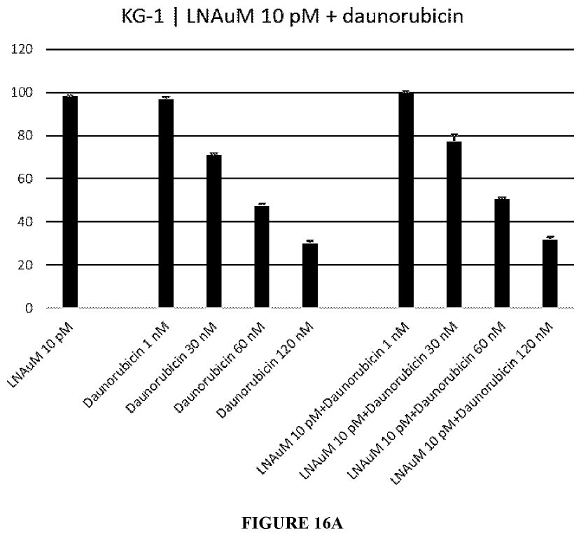

KG-1 cells were maintained as above. Cells were seeded 15 000 cells/well to 96-well plate at volume of 50 μl. KG-1 cells were treated with 10 pM, 500 pM, 2 nM, or 8 nM of LNAuM 10 in the presence or absence of cytarabine. Cytarabine was tested at concentrations 0.67, 73.3, 220 and 440 nM. Dilutions (á 25 μl) of LNAuM or cytarabine were added as 4-fold concentrations to cells. Each 96-well plate had its' own small molecule, cell and medium controls. Incubation time was 4 days. The viability of the cells was determined as above. Cytarabine results are shown on FIG. 15.

TABLE 2-3
n-fold change and % decrease as to combination, KG-1 cells.
Bold indicate more than 40% reduction. Strong reduction in
cell survival is seen in certain cytarabine concentrations.

| | | | | LNAuM % | Drug % |
|---|---|---|---|---|---|
| LNAuM | Cytarabine | LNAuM | Drug | decrease | decrease |
| concen- | concen- | fold to | fold to | to | to |
| tration | tration | combi- | combi- | combi- | combi- |
| (nM) | (nM) | nation | nation | nation | nation |
| 0.01 | 0.67 | 1.0 | 1.0 | 0.8% | −2.0% |
| 0.01 | 73.3 | 1.3 | 1.1 | 20.1% | 5.2% |
| 0.01 | 220 | 2.0 | 1.0 | 49.7% | 3.6% |
| 0.01 | 440 | 4.0 | 1.0 | 75.0% | 2.0% |
| 0.5 | 0.67 | 1.1 | 1.3 | 8.7% | 22.7% |
| 0.5 | 73.3 | 5.0 | 5.1 | 80.1% | 80.5% |
| 0.5 | 220 | 3.5 | 2.2 | 71.7% | 55.3% |
| 0.5 | 440 | 3.8 | 1.2 | 73.9% | 15.6% |
| 2 | 0.67 | 1.1 | 1.9 | 9.4% | 47.5% |
| 2 | 73.3 | 4.0 | 5.9 | 74.9% | 83.2% |
| 2 | 220 | 2.3 | 2.1 | 56.6% | 53.1% |
| 2 | 440 | 2.3 | 1.0 | 56.2% | 2.9% |
| 8 | 0.67 | 1.1 | 3.0 | 12.8% | 67.0% |
| 8 | 73.3 | 3.0 | 6.9 | 66.6% | 85.4% |
| 8 | 220 | 1.6 | 2.2 | 36.7% | 55.4% |
| 8 | 440 | 1.6 | 1.1 | 35.8% | 7.3% |

Combination Study of LNAuM with Daunorubicin

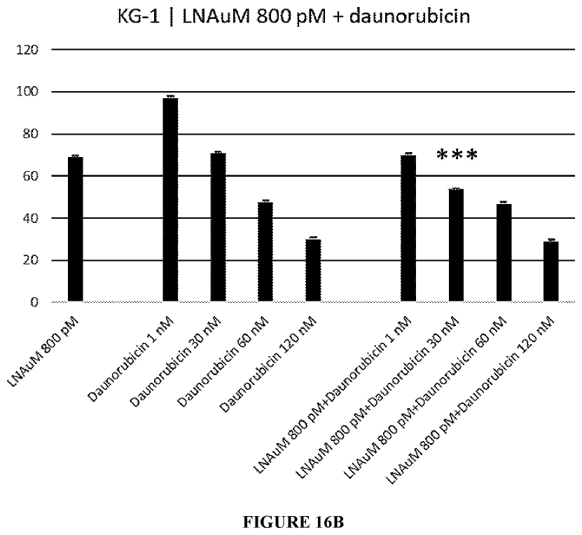

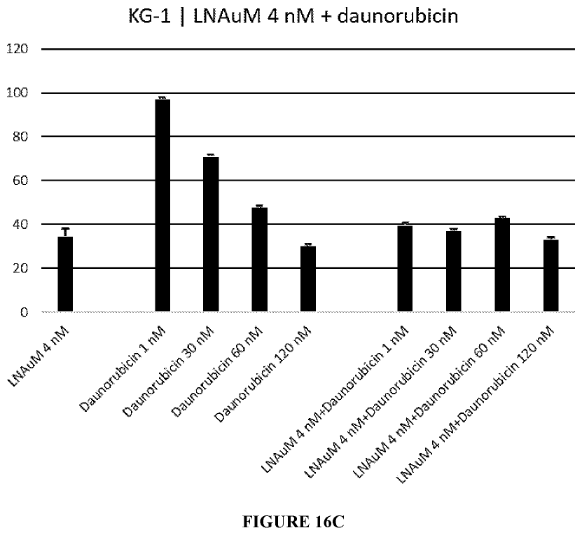

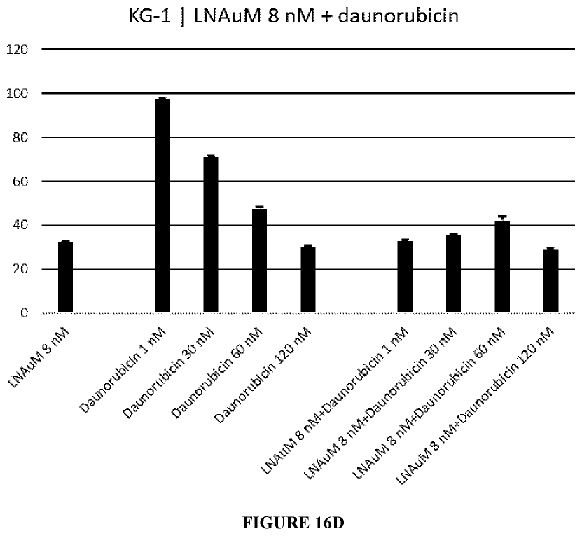

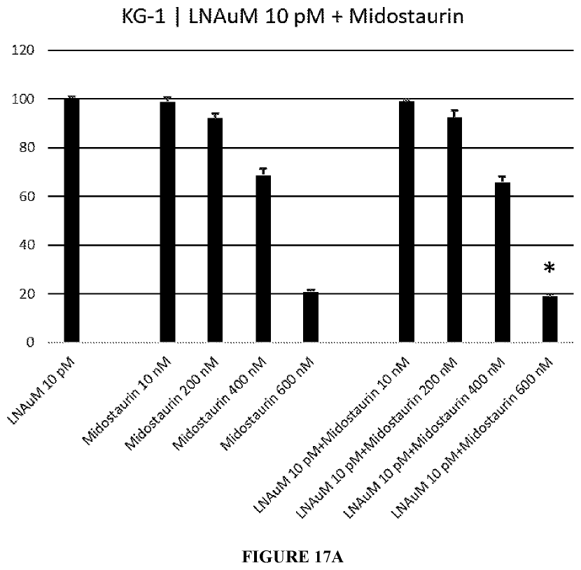

KG-1 cells were maintained as above. Cells were seeded 15 000 cells/well to 96-well plate at volume of 50 μl. KG-1 cells were treated with 10 pM, 800 pM, 4 nM, or 8 nM of LNAuM in the presence or absence of daunorubicin. Daunorubicin was tested at concentrations 1, 30, 60 and 120 nM. Dilutions (á 25 μl) of LNAuM or daunorubicin were added as 4-fold concentrations to cells. Each 96-well plate had its' own small molecule, cell and medium controls. Incubation time was 4 days. The viability of the cells was determined as described above. Daunorubicin results are shown on FIG. 16.

Combination Study of LNAuM with Midostaurin

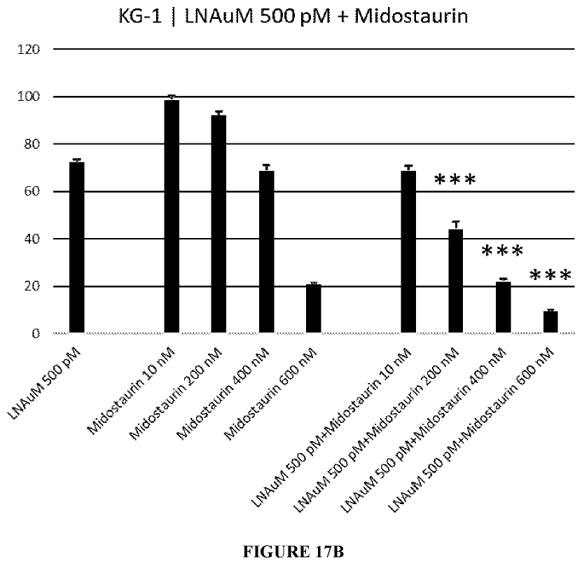

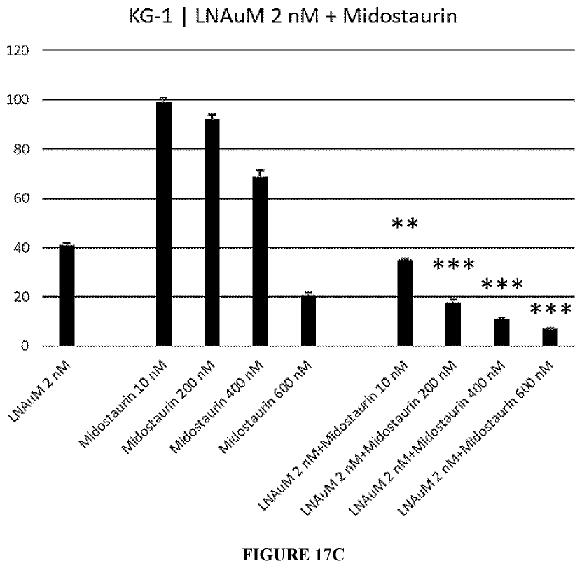

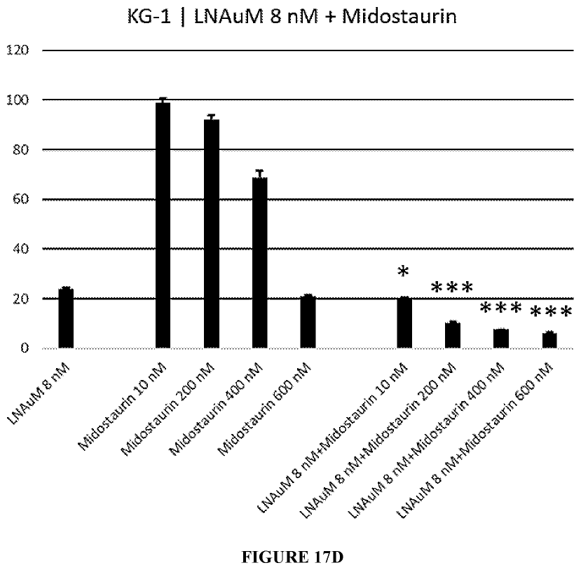

KG-1 cells were maintained as above. Cells were seeded 15 000 cells/well to 96-well plate at volume of 50 μl. KG-1 cells were treated with 10 pM, 500 pM, 2 nM, or 8 nM of LNAuM in the presence or absence of midostaurin. Midostaurin was tested at concentrations 10, 200, 400 and 600 nM. Dilutions (á 25 μl) of LNAuM or midostaurin were added as 4-fold concentrations to cells. Each 96-well plate had its' own small molecule, cell and medium controls. Incubation time was 4 days. The viability of the cells was determined as above. Midostaurin results are shown on FIG. 17.

TABLE 2-4
n-fold change and % decrease as to combination, KG-1
cells. Bold indicate more than 40% reduction.

| | | | | LNAuM % | Drug % |
|---|---|---|---|---|---|
| LNAuM | Midostaurin | LNAuM | Drug | decrease | decrease |
| concen- | concen- | fold to | fold to | to | to |
| tration | tration | combi- | combi- | combi- | combi- |
| (nM) | (nM) | nation | nation | nation | nation |
| 0.01 | 10 | 1.0 | 1.0 | 1.5% | −0.2% |
| 0.01 | 200 | 1.1 | 1.0 | 8.0% | −0.4% |
| 0.01 | 400 | 1.5 | 1.0 | 34.6% | 4.4% |
| 0.01 | 600 | 5.3 | 1.1 | 81.0% | 8.5% |
| 0.5 | 10 | 1.1 | 1.4 | 5.0% | 30.4% |
| 0.5 | 200 | 1.6 | 2.1 | 39.0% | 52.1% |
| 0.5 | 400 | 3.3 | 3.1 | 69.5% | 68.0% |
| 0.5 | 600 | 7.8 | 2.2 | 87.2% | 55.5% |
| 2 | 10 | 1.2 | 2.8 | 14.8% | 64.6% |
| 2 | 200 | 2.3 | 5.1 | 56.2% | 80.5% |
| 2 | 400 | 3.7 | 6.3 | 73.3% | 84.1% |
| 2 | 600 | 5.8 | 2.9 | 82.7% | 66.0% |
| 8 | 10 | 1.2 | 4.9 | 15.7% | 79.7% |
| 8 | 200 | 2.3 | 8.9 | 56.6% | 88.8% |
| 8 | 400 | 3.1 | 9.1 | 68.2% | 89.0% |
| 8 | 600 | 3.9 | 3.4 | 74.2% | 70.5% |

Table 2-4 and FIG. 17 show that LNAuM+midostaurin combinations at concentrations 500 pM+400/600 pM and 2 nM/8 nM+200/400/600 nM pairings reduce KG-1 cell viability more than 40% compared to single agent cell survival indicating very strong synergistic effect.

Combination Study of LNAuM with Gilteritinib

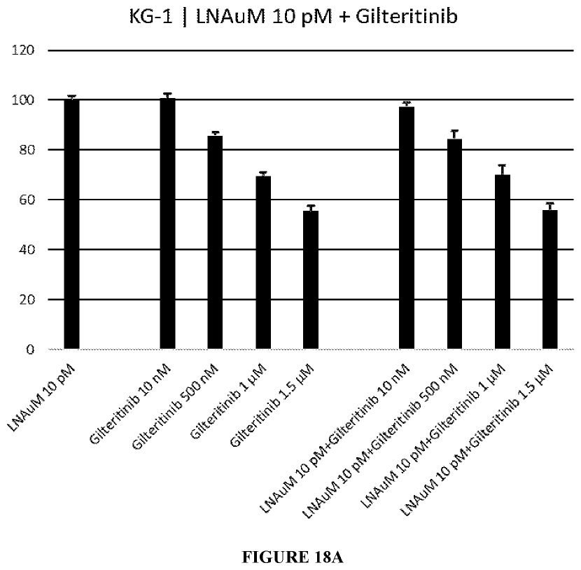

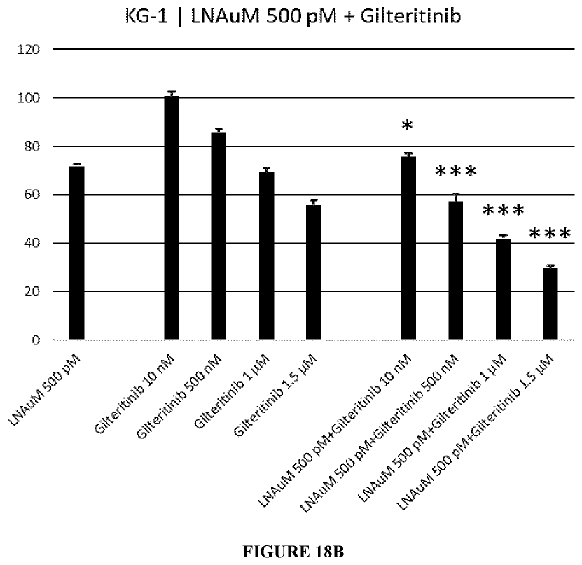

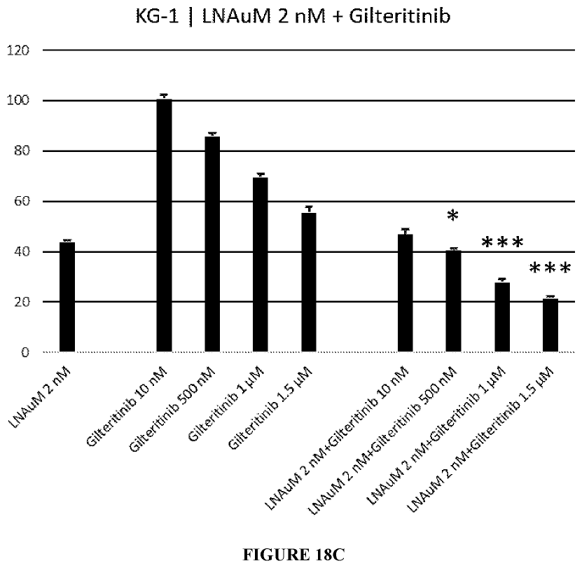

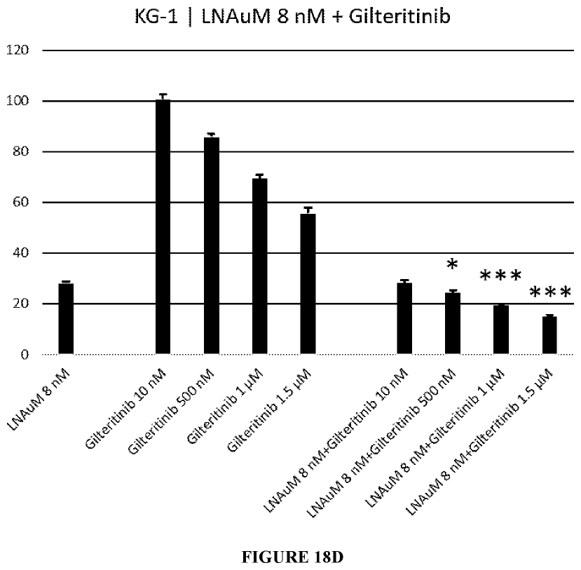

KG-1 cells were maintained as above. Cells were seeded 15 000 cells/well to 96-well plate at volume of 50 μl. KG-1 cells were treated with 10 pM, 500 pM, 2 nM, or 8 nM of LNAuM in the presence or absence of gilteritinib. Gilteritinib was tested at concentrations 10 nM, 500, 1000 and 1500 nM. Dilutions (a 25 μl) of LNAuM or gilteritinib were added as 4-fold concentrations to cells. Each 96-well plate had its' own small molecule, cell and medium controls. Incubation time was 4 days. The viability of the cells was determined as Example 1. Gilteritinib results are presented on FIG. 18.

TABLE 2-5
n-fold change and % decrease as to combination, KG-1
cells. Bold indicate more than 40% reduction.

| | | | | LNAuM % | Drug % |
|---|---|---|---|---|---|
| LNAuM | Gilteritinib | LNAuM | Drug | decrease | decrease |
| concen- | concen- | fold to | fold to | to | to |
| tration | tration | combi- | combi- | combi- | combi- |
| (nM) | (nM) | nation | nation | nation | nation |
| 0.01 | 10 | 1.0 | 1.0 | 2.6% | 3.3% |
| 0.01 | 500 | 1.2 | 1.0 | 15.5% | 1.5% |
| 0.01 | 1000 | 1.4 | 1.0 | 29.9% | −1.0% |
| 0.01 | 1500 | 1.8 | 1.0 | 44.1% | −0.6% |
| 0.5 | 10 | 0.9 | 1.3 | −5.4% | 24.8% |
| 0.5 | 500 | 1.3 | 1.5 | 20.3% | 33.2% |
| 0.5 | 1000 | 1.7 | 1.7 | 41.7% | 39.7% |
| 0.5 | 1500 | 2.4 | 1.9 | 58.7% | 46.6% |
| 2 | 10 | 0.9 | 2.1 | −6.9% | 53.5% |
| 2 | 500 | 1.1 | 2.1 | 7.8% | 52.8% |
| 2 | 1000 | 1.6 | 2.5 | 36.6% | 60.0% |
| 2 | 1500 | 2.0 | 2.6 | 50.9% | 61.3% |
| 8 | 10 | 1.0 | 3.6 | −0.8% | 72.0% |
| 8 | 500 | 1.2 | 3.5 | 13.2% | 71.6% |
| 8 | 1000 | 1.5 | 3.6 | 31.3% | 72.3% |
| 8 | 1500 | 1.9 | 3.7 | 46.3% | 72.9% |

Table 2-5 and FIG. 18 show that LNAuM+gilteritinib combinations at concentrations 500 pM/2 nM/8 nM+500 nM/1 μM/1.5 μM pairings reduce KG-1 cell viability compared to single agent cell survival indicating synergistic effect.

### Synergy Score Calculations

Zip synergy scores were calculated as described in Example 1 and results are shown in Table 2-6.

TABLE 2-6
Summary of ZIP synergy scores per combination. Venetoclax
shows synergistic effect with LNAuM while cytarabine, midostauring
and gilteritinib effect is at least additive.

| Assay | ZIP Score |
|---|---|
| LNAuM + cytarabine | 9.4 (most synergistic area score 15.3) |
| LNAuM + venetoclax | 14.9 (most synergistic area score 25.6) |
| LNAuM + daunorubicin | −8.6 (most synergistic area score −0.95) |
| LNAuM + midostaurin | 9.6 (most synergistic area score 15.5) |
| LNAuM + gilteritinib | 1.9 (most synergistic area score 3.2) |

### Example 3—MOLM-13 Cells

MOLM-13 cells (German Collection of Microorganisms and Cell Cultures, cell line ACC 554), derived from human leukemia, were cultured in RPMI 1640 Medium (Gibco, Life Technologies) supplemented with 10% heat inactivated FBS (Gibco, Life Technologies). Cells were maintained in a humidified atmosphere containing 5% CO2 at +37° C.

For toxicity assay, MOLM-13 cells were seeded for 10 000 cells/well of 96-well plate. The tested chemotherapeutic agents were added as 2-fold stock solutions and incubated in duplicate with cells for 4 days. The compounds were tested at concentrations 0.1, 1, 10, 100, 1000, and 10 000 nM. Due to the limited solubility the highest concentration of gilteritinib was 2000 nM. After 4 days incubation, the viability of the cells was determined with PrestoBlue Cell Viability Reagent (Life Technologies) according to the manufacturer's instructions. The percentage of viable cells was calculated by dividing the sample absorbance value with untreated cell control value and multiplying by 100%. IC50 values were determined as described in Example 1. The IC50 values are shown in Table 3-1.

TABLE 3-1
IC50 values of MOLM-13 cells per chemotherapeutic agent.

| Compound | IC50 |
|---|---|
| Cytarabine | 112 nM |
| Venetoclax | 24 nM |
| Daunorubicin-HCl | 13 nM |
| Midostaurin | 14 nM |
| Gilteritinib hemifumarate | 24 nM |

Combination Study of LNAuM with Cytarabine or Venetoclax

MOLM-13 cells were maintained as described above. Cells were seeded (20 000 cells/well) to non-treated 96-well plate at volume of 50 μl. MOLM-13 cells were treated with 0.001, 0.05, 0.25 or 5 nM LNAuM in the presence or absence of cytarabine or venetoclax. Cytarabine was tested at concentrations 1, 50, 100 or 200 nM and venetoclax was tested at concentrations 1, 10, 25 and 70 nM. Dilutions (á 25 μl) of LNAuM, cytarabine or venetoclax were added as 4-fold concentrations. Each 96-well plate had its' own small molecule, cell and medium controls. Incubation time was 4 days.

The viability of the cells was determined with PrestoBlue Cell Viability Reagent (Life Technologies) according to the manufacturer's instructions. The percentage of viable cells was calculated by dividing the sample absorbance value with untreated cell control value and multiplying by 100. The viability percentages for cytarabine and venetoclax alone, without LNAuM, were calculated as averages of two dilution series (from two 96-well plates). For statistical analysis and synergy score calculations the cell control values were calculated as averages of the two 96-well plates.

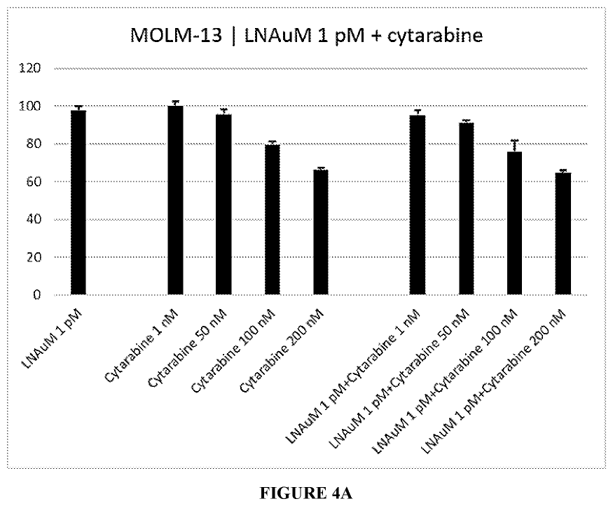

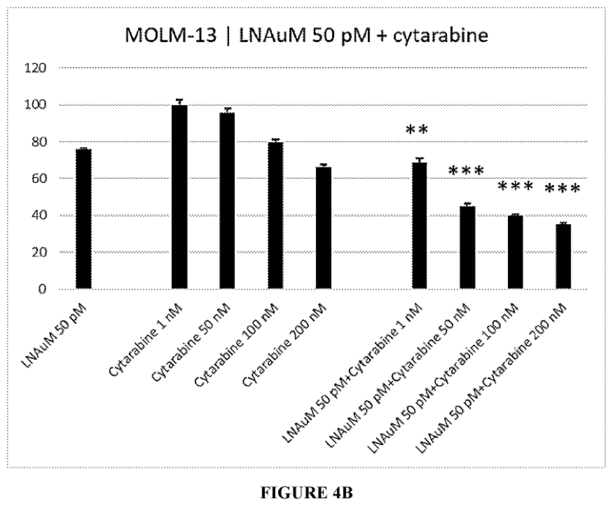

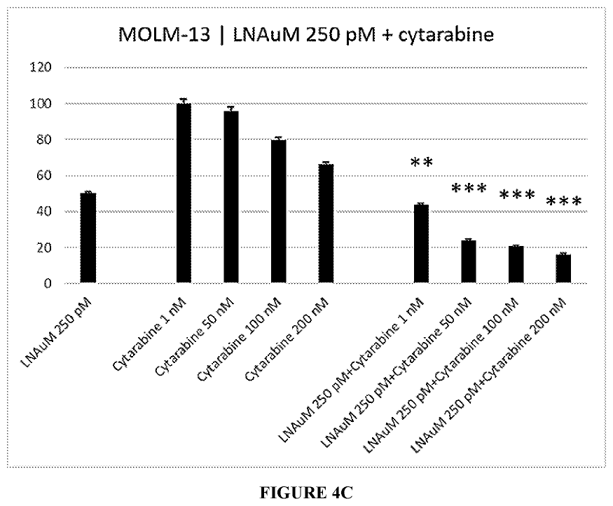

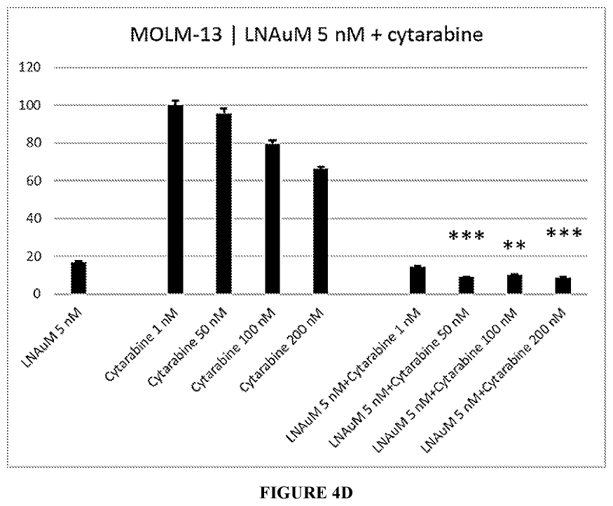

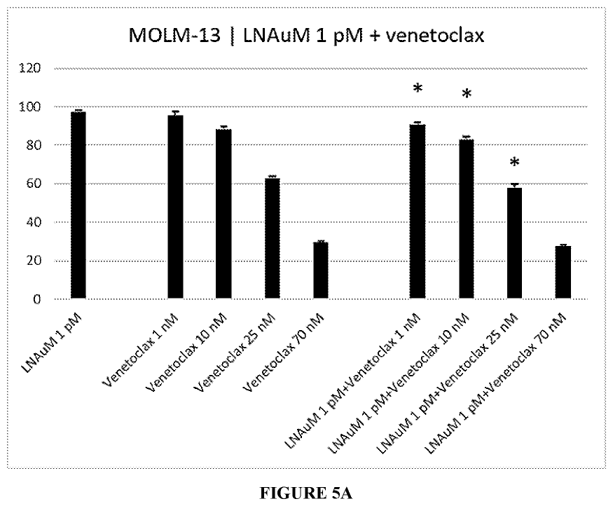

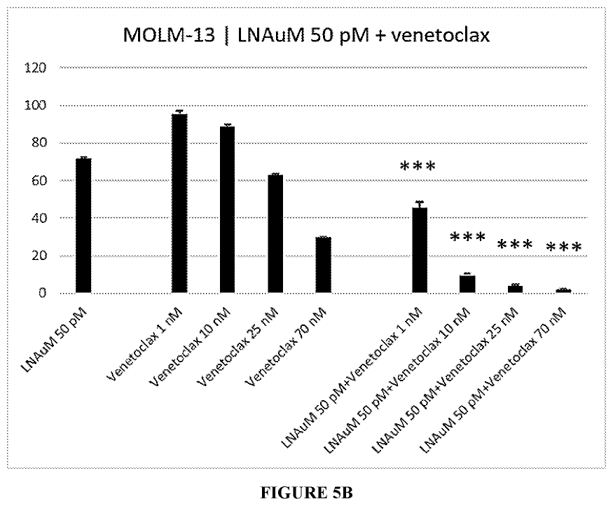

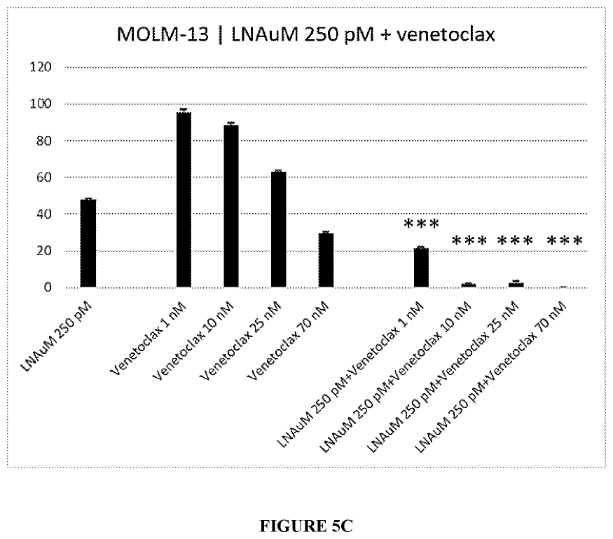

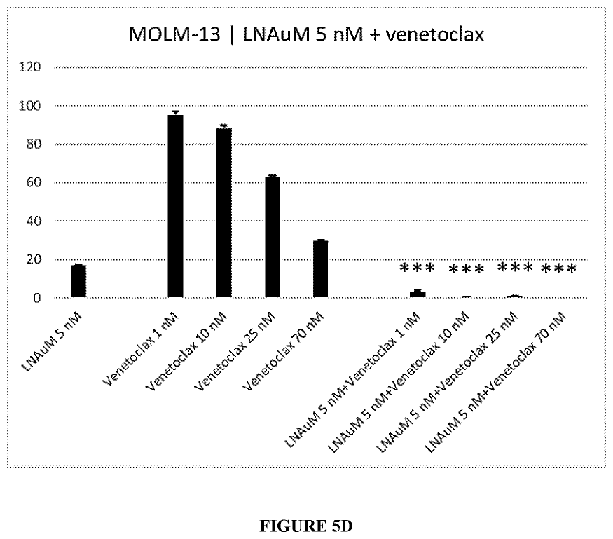

Results are shown in FIGS. 4 and 5.

Figures and Table 3-2 show that LNAuM+cytarabine combinations at concentrations 250 pM of LNAuM and 50-200 nM of cytarabine reduce MOLM-13 cell viability more than 50% compared to single agent cell survival (bold in Table 3-2) indicating strong synergistic effect. The strong synergy is started to be seen already in 50 pM concentration of LNAuM and 50-200 nM of cytarabine and is seen also in highest tested concentration of LNAuM (5 nM) (underlined).

Figures and Table 3-3 show that reduction of all LNAuM+venetoclax combinations are statistically significant compared to their single compound data (except combination LNAuM 1 pM+venetoclax 70 nM). Concentrations 50 pM-5 nM of LNAuM and 10-70 nM of venetoclax reduce MOLM-13 cell viability more than 50% compared to single agent cell survival (Table 3-3, bold) indicating strong synergistic effect. Combination of 50 pM of LNAuM and 1 nM of venetoclax produces already a strong synergistic effect.

TABLE 3-2
n-fold change and % decrease as to combination (LNAuM and
cytarabine). N %, decrease in cell survival >50%;
n %, decrease in cell survival between 40 and 50%.

| | | | | LNAuM % | Drug % |
|---|---|---|---|---|---|
| LNAuM | Cytarabine | LNAuM | Drug | decrease | decrease |
| concen- | concen- | fold to | fold to | to | to |
| tration | tration | combi- | combi- | combi- | combi- |
| (pM) | (pM) | nation | nation | nation | nation |
| 1 | 1000 | 1.0 | 1.1 | 2.6% | 4.8% |
| 1 | 50000 | 1.1 | 1.1 | 6.8% | 4.8% |
| 1 | 100000 | 1.3 | 1.0 | 22.4% | 4.6% |
| 1 | 200000 | 1.5 | 1.0 | 34.0% | 2.3% |
| 50 | 1000 | 1.1 | 1.5 | 9.7% | 31.5% |
| 50 | 50000 | 1.7 | 2.1 | 40.9% | 53.1% |
| 50 | 100000 | 1.9 | 2.0 | 47.4% | 49.8% |
| 50 | 200000 | 2.2 | 1.9 | 53.6% | 46.7% |
| 250 | 1000 | 1.1 | 2.3 | 12.4% | 56.1% |
| 250 | 50000 | 2.1 | 4.0 | 52.5% | 75.1% |
| 250 | 100000 | 2.4 | 3.8 | 58.4% | 73.8% |
| 250 | 200000 | 3.1 | 4.1 | 67.5% | 75.3% |
| 5000 | 1000 | 1.2 | 6.9 | 13.8% | 85.6% |
| 5000 | 50000 | 1.9 | 10.7 | 46.8% | 90.7% |
| 5000 | 100000 | 1.6 | 7.7 | 38.5% | 87.0% |
| 5000 | 200000 | 1.9 | 7.6 | 48.1% | 86.8% |

TABLE 3-3
n-fold change and % decrease as to combination (LNAuM and
venetoclax). N %, decrease in cell survival >50%;
n %, decrease in cell survival between 40 and 50%.

| | | | | LNAuM % | Drug % |
|---|---|---|---|---|---|
| LNAuM | Venetoclax | LNAuM | Drug | decrease | decrease |
| concen- | concen- | fold to | fold to | to | to |
| tration | tration | combi- | combi- | combi- | combi- |
| (pM) | (pM) | nation | nation | nation | nation |
| 1 | 1000 | 1.1 | 1.1 | 6.7% | 4.8% |
| 1 | 10000 | 1.2 | 1.1 | 14.5% | 6.0% |
| 1 | 25000 | 1.7 | 1.1 | 40.4% | 7.9% |
| 1 | 70000 | 3.5 | 1.1 | 71.8% | 7.3% |
| 50 | 1000 | 1.6 | 2.1 | 36.7% | 52.4% |
| 50 | 10000 | 7.5 | 9.3 | 86.7% | 89.2% |
| 50 | 25000 | 17.1 | 14.9 | 94.1% | 93.3% |
| 50 | 70000 | 38.5 | 15.9 | 97.4% | 93.7% |
| 250 | 1000 | 2.2 | 4.5 | 55.1% | 77.6% |
| 250 | 10000 | 25.6 | 47.6 | 96.1% | 97.9% |
| 250 | 25000 | 18.7 | 24.7 | 94.7% | 96.0% |
| 250 | 70000 | −134.8 | −84.0 | 100.7% | 101.2% |
| 5000 | 1000 | 4.9 | 27.9 | 79.7% | 96.4% |
| 5000 | 10000 | 72.8 | 381.1 | 98.6% | 99.7% |
| 5000 | 25000 | 16.9 | 62.8 | 94.1% | 98.4% |
| 5000 | 70000 | −56.7 | −99.4 | 101.8% | 101.0% |

Combination Study of LNAuM with Midostaurin or Gilteritinib Hemifumarate

MOLM-13 cells were maintained as described above. Cells were seeded (20 000 cells/well) to non-treated 96-well plate at volume of 50 μl. MOLM-13 cells were treated with 0.001, 0.05, 0.25 or 5 nM of LNAuM in the presence or absence of midostaurin or gilteritinib hemifumarate. Midostaurin was tested at concentrations 1, 5, 15 or 25 nM and gilteritinib hemifumarate was tested at concentrations 1, 15, 25 and 40 nM. Dilutions (á 25 μl) of LNAuM, midostaurin or gilteritinib hemifumarate were added as 4-fold concentrations. Each 96-well plate had its' own small molecule, cell and medium controls. Incubation time was 4 days.

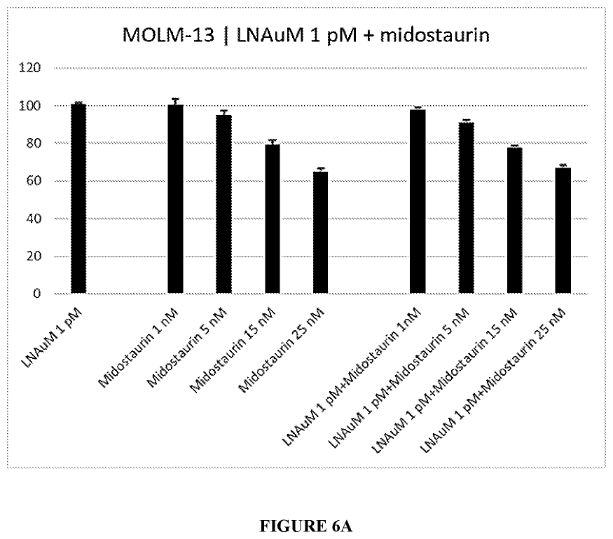

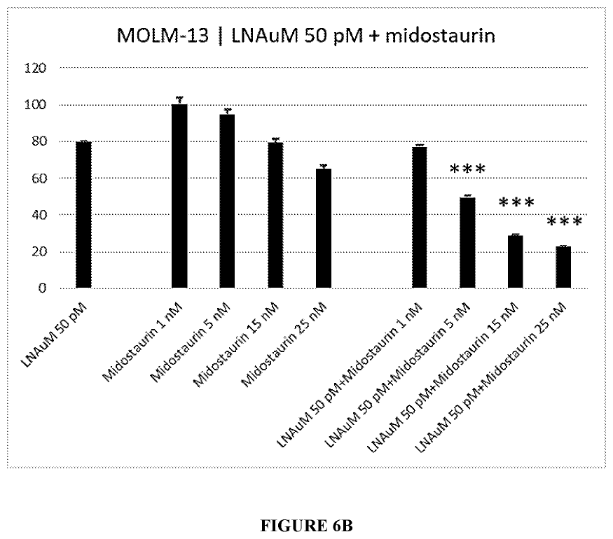

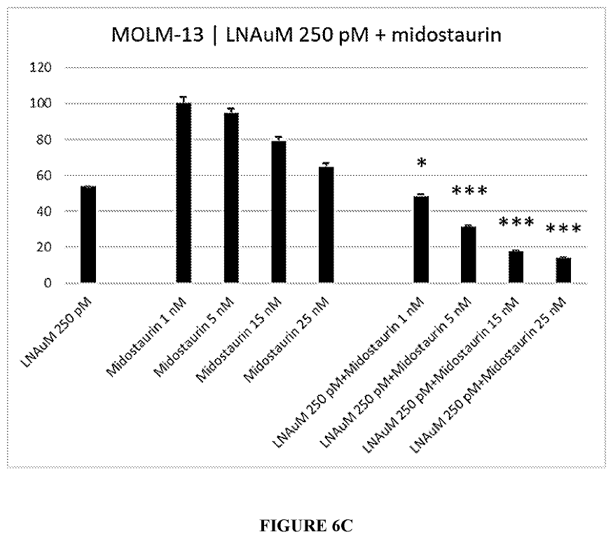

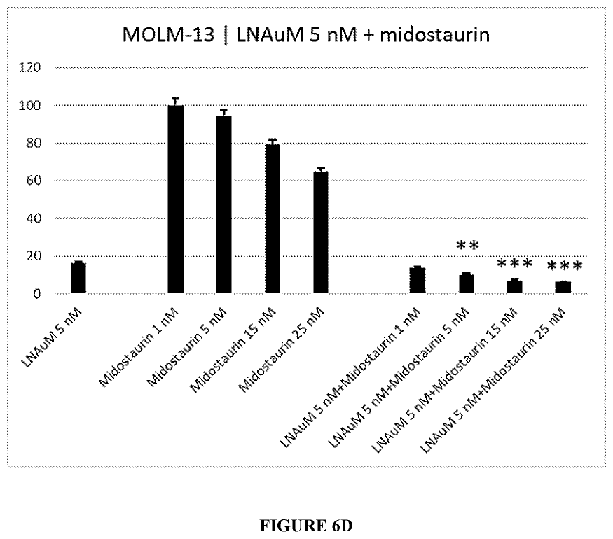

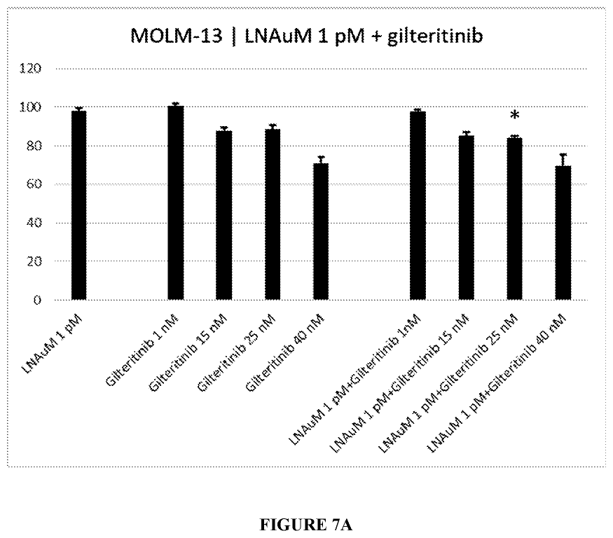

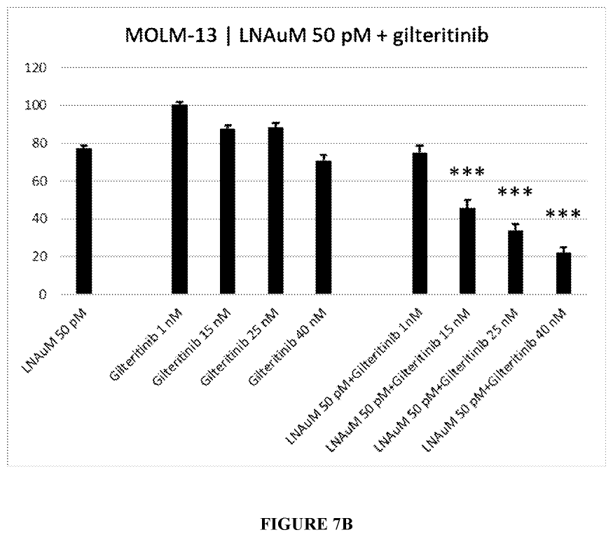

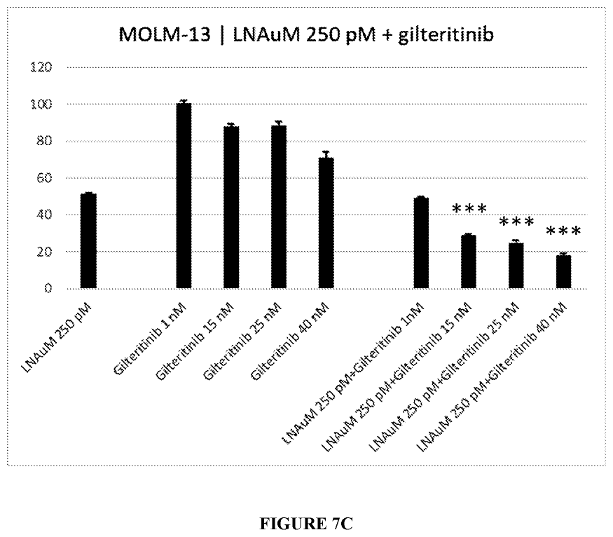

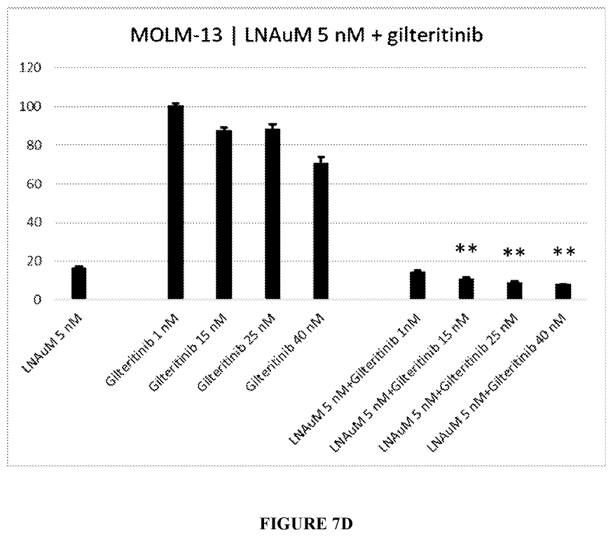

The viability of the cells was determined with PrestoBlue Cell Viability Reagent (Life Technologies) according to the manufacturer's instructions. The percentage of viable cells was calculated by dividing the sample absorbance value with untreated cell control value from the same 96-well plate and multiplying by 100. The viability percentages for midostaurin and gilteritinib hemifumarate alone, without LNAuM, were calculated as averages of two dilution series (from two 96-well plates). For statistical analysis and synergy score calculations the cell control values were calculated as averages of the two 96-well plates. Results are shown in FIGS. 6 and 7.

FIG. 6 and Table 3-4 show that LNAuM+midostaurin combinations at concentrations 50 pM-5 nM of LNAuM and 15-25 nM of midostaurin reduce MOLM-13 cell viability more than 50% compared to single agent cell survival (Table 3-4 bold) indicating strong synergistic effect. The strong synergy is started to be seen already in 50 pM or 250 pM concentrations of LNAuM combined with 5 nM of midostaurin.

FIG. 7 and Table 3-5 show that almost all LNAuM+gilteritinib combinations at concentrations 50 pM-5 nM of LNAuM and 25-40 nM of gilteritinib reduce MOLM-13 cell viability more than 50% compared to single agent cell survival (bold) indicating strong synergistic effect. The strong synergy is started to be seen already in 50 pM or 250 pM concentrations of LNAuM combined with 15 nM of gilteritinib.

TABLE 3-4
n-fold change and % decrease as to combination (LNAuM and
midostaurin). N %, decrease in cell survival >50%;
n %, decrease in cell survival between 40 and 50%.

| | | | | LNAuM % | Drug % |
|---|---|---|---|---|---|
| LNAuM | Midostaurin | LNAuM | Drug | decrease | decrease |
| concen- | concen- | fold to | fold to | to | to |
| tration | tration | combi- | combi- | combi- | combi- |
| (pM) | (pM) | nation | nation | nation | nation |
| 1 | 1000 | 1.0 | 1.0 | 2.8% | 2.2% |
| 1 | 5000 | 1.1 | 1.0 | 9.7% | 4.1% |
| 1 | 15000 | 1.3 | 1.0 | 22.7% | 1.8% |
| 1 | 25000 | 1.5 | 1.0 | 33.5% | −3.2% |
| 50 | 1000 | 1.0 | 1.3 | 3.6% | 23.1% |
| 50 | 5000 | 1.6 | 1.9 | 38.1% | 47.9% |
| 50 | 15000 | 2.8 | 2.7 | 63.9% | 63.6% |
| 50 | 25000 | 3.5 | 2.9 | 71.7% | 65.2% |
| 250 | 1000 | 1.1 | 2.1 | 10.1% | 51.7% |
| 250 | 5000 | 1.7 | 3.0 | 41.3% | 66.7% |
| 250 | 15000 | 3.1 | 4.5 | 67.4% | 77.9% |
| 250 | 25000 | 3.9 | 4.7 | 74.1% | 78.6% |
| 5000 | 1000 | 1.2 | 7.3 | 14.1% | 86.3% |
| 5000 | 5000 | 1.6 | 9.7 | 38.9% | 89.7% |
| 5000 | 15000 | 2.3 | 11.2 | 56.0% | 91.1% |
| 5000 | 25000 | 2.6 | 10.6 | 61.7% | 90.6% |

TABLE 3-5
n-fold change and % decrease as to combination (LNAuM and
gilteritinib). N %, decrease in cell survival >50%;
n %, decrease in cell survival between 40 and 50%.

| | | | | LNAuM % | Drug % |
|---|---|---|---|---|---|
| LNAuM | Gilteritinib | LNAuM | Drug | decrease | decrease |
| concen- | Concen- | fold to | fold to | to | to |
| tration | tration | combi- | combi- | combi- | combi- |
| (pM) | (pM) | nation | nation | nation | nation |
| 1 | 1000 | 1.0 | 1.0 | 0.2% | 2.6% |
| 1 | 15000 | 1.1 | 1.0 | 13.0% | 3.0% |
| 1 | 25000 | 1.2 | 1.1 | 14.3% | 5.0% |
| 1 | 40000 | 1.4 | 1.0 | 28.8% | 1.6% |
| 50 | 1000 | 1.0 | 1.3 | 3.0% | 25.3% |
| 50 | 15000 | 1.7 | 1.9 | 40.9% | 48.0% |
| 50 | 25000 | 2.3 | 2.6 | 56.3% | 61.7% |
| 50 | 40000 | 3.5 | 3.2 | 71.4% | 68.8% |
| 250 | 1000 | 1.0 | 2.0 | 4.7% | 51.2% |
| 250 | 15000 | 1.8 | 3.0 | 43.7% | 67.0% |
| 250 | 25000 | 2.1 | 3.5 | 51.6% | 71.8% |
| 250 | 40000 | 2.9 | 4.0 | 65.3% | 74.8% |
| 5000 | 1000 | 1.1 | 6.9 | 11.6% | 85.4% |
| 5000 | 15000 | 1.5 | 8.1 | 34.6% | 87.6% |
| 5000 | 25000 | 1.8 | 9.8 | 45.5% | 89.8% |
| 5000 | 40000 | 2.1 | 8.8 | 51.6% | 88.7% |

Combination Study of LNAuM with Daunorubicin

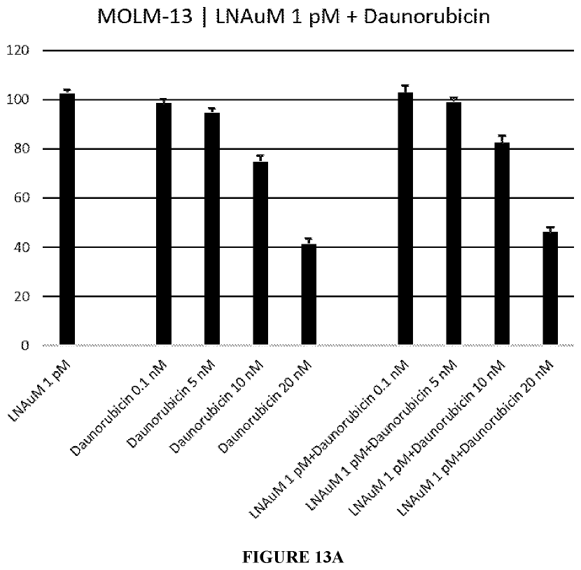

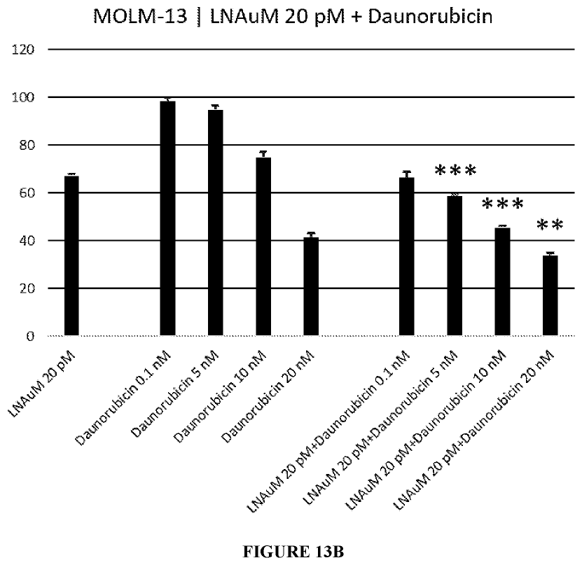

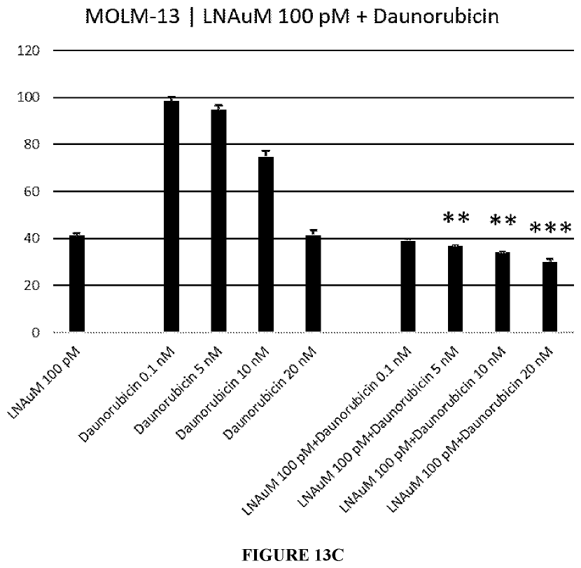

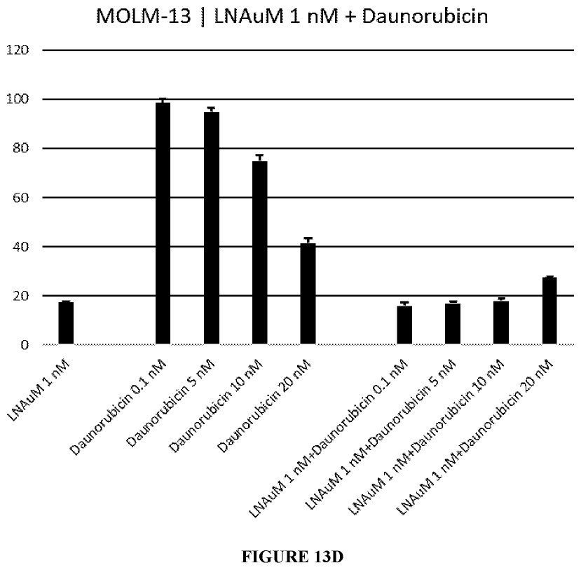

MOLM-13 cells were maintained as described above. Cells were seeded (20 000 cells/well) to non-treated 96-well plate at volume of 50 μl. MOLM-13 cells were treated with 0.001, 0.02, 0.1 or 1 nM of LNAuM in the presence or absence of daunorubicin. Daunorubicin was tested at concentrations 0.1, 5, 10 or 20 nM. Dilutions (á 25 μl) of LNAuM and daunorubicin were added as 4-fold concentrations to cells. Each 96-well plate had its' own small molecule, cell and medium controls. Incubation time was 4 days. The viability of the cells was determined with PrestoBlue Cell Viability Reagent as above. The viability percentages for daunorubicin alone, without LnAuM, were calculated as averages of two dilution series (from two 96-well plates). For statistical analysis and synergy score calculations the cell control values were calculated as averages of the two 96-well plates. Results are shown in FIG. 13.

### Synergy Score Calculations

Zip synergy scores were calculated as described in Example 1 and results are shown in Table 3-6. Before calculations, concentrations were transformed into pM.

TABLE 3-6
Summary of ZIP synergy scores per combination in MOLM-13 cells.
Venetoclax, midostauring and gilteritinib show synergistic
effect while cytarabine effect is at least additive.

| Assay | ZIP Score |
|---|---|
| LNAuM + cytarabine | 8.5 (most synergistic area score 15.0) |
| LNAuM + venetoclax | 21.3 (most synergistic area score 29.6) |
| LNAuM + midostaurin | 12.5 (most synergistic area score 21.4) |
| LNAuM + gilteritinib | 12.5 (most synergistic area score 22.0) |
| LNAuM + daunorubicin | −0.17 (most synergistic area score 2.45) |

### Example 4—MOLM-13 Cell Mouse Xenografts

MOLM-13 cells (German Collection of Microorganisms and Cell Cultures, cell line ACC 554), derived from human leukemia, were cultured in RPMI 1640 Medium (Gibco, Life Technologies) supplemented with 10% heat inactivated FBS (Gibco, Life Technologies). Cells were maintained at 0.1-2.0×10⁶ cells/ml, splitting every 3-4 days, in a humidified atmosphere containing 5% CO₂ at +37° C.

On the day of inoculation, the cells were harvested and pelleted by centrifugation at 200×g for 5 minutes. Cells were then suspended in RPMI 1640 medium and the viability was counted with Trypan blue using a hemocytometer. Cells were then diluted with RPMI 1640 medium to a density of 40×106 viable cells/ml and aliquoted á 0.75 ml to Eppendorf tubes and stored on ice until inoculated. Before inoculation equal volume of Matrigel (BD Biosciences) was added to cell suspension. Final cell density was 20×10⁶ cells/ml of which 100 μl (2×10⁶ cells) was inoculated subcutaneously to each mouse.

In vivo anti-leukemia xenograft efficacy was evaluated for LNAuM (DAR≈7.8) and venetoclax separately and in combination. The study was performed at the TCDM/Central Animal Laboratory, University of Turku, Finland, according to the appropriate ethical committee approval. Cells for inoculation to mice were prepared in vigorous exponential growth phase. 2 million MOLM-13 cells in 50% Matrigel were inoculated s.c. to the flank of each mouse (female athymic nude mice between 8-10 weeks of age). Clinical signs and general behavior of the animals were observed regularly. No signs of toxicity were recorded. At the end of the study, the mice were examined for potential macroscopic changes in major organs, but none were detected. Tumor growth was followed by palpation. After caliper measurement, tumor volume was calculated according to 0.5×length×width².

Venetoclax was dosed via oral gavage (p.o.). Venetoclax suspensions were prepared on the day of dosing by adding the following components stepwise, with vortexing, to a tube containing 85 mg venetoclax (MedChemExpress) in 425 μl DMSO: 4.25 ml PEG300, 425 μl Tween 80, and 3.4 ml deionized water. The first dosings were administered when average tumor volume reached 120 mm³. Mice were evenly divided into study groups, 6 mice in each group, so that each group received similar distribution of different-sized tumors and the average tumor volumes were similar in each group. Intravenous (i.v.) treatment of the ADC in PBS was given once on day 1 of treatment as a single dose regimen (on day 8 after tumor inoculation), while altogether 10 daily doses of venetoclax were given on days 1-9 and day 12 of treatment (on days 8-17 and day 20 after tumor inoculation). The experiment lasted to day 46 after treatment (day 54 after tumor inoculation).

Serum samples were taken from the animals at time points after the i.v. administration and human antibody levels were determined with anti-human IgG ELISA assays (Human IgG Quantification Kit, RD-Biotech). Based on the ADC exposures determined with the ELISA assays, one mouse in the venetoclax+LNAuM 1 mg/kg combination group had not received the intended ADC dose and was removed from the study group.

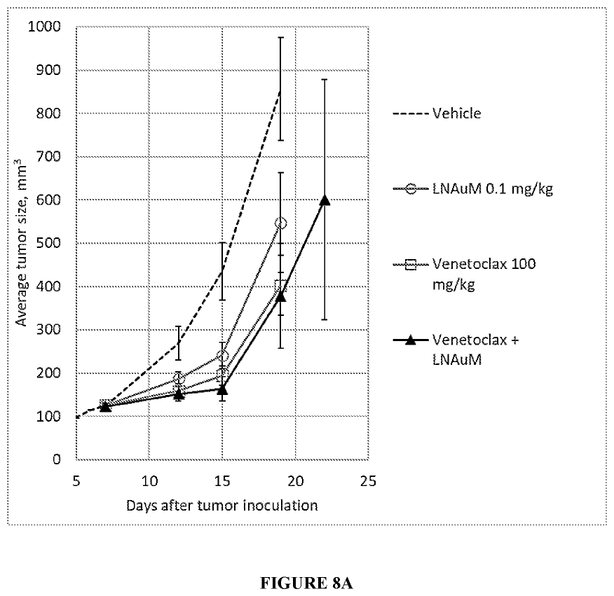

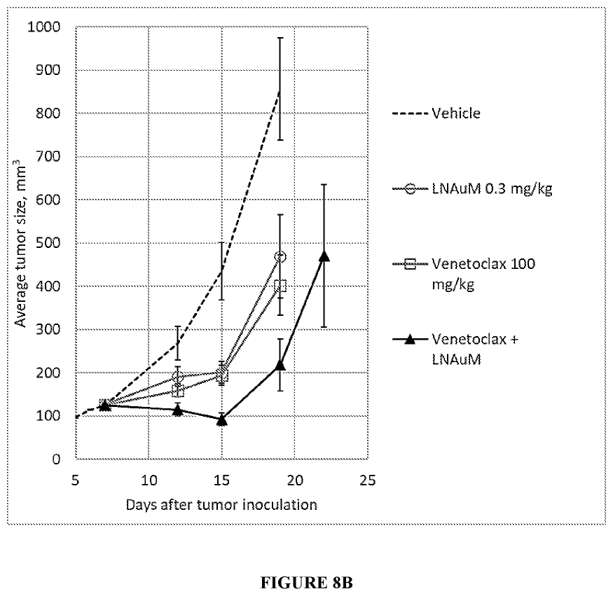

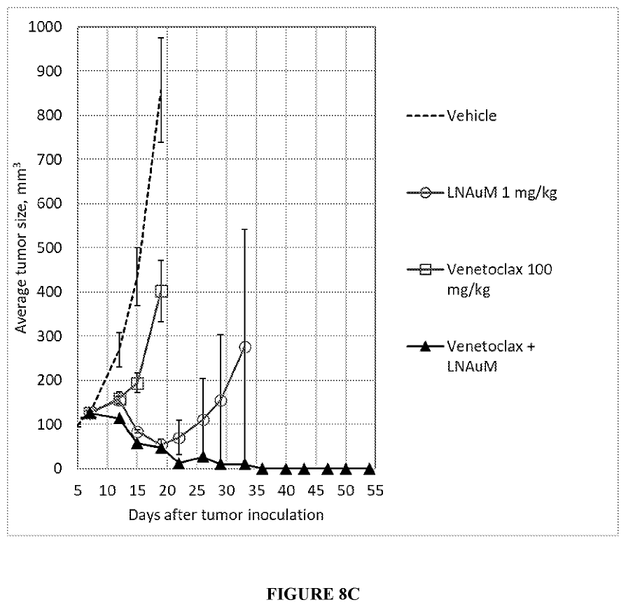

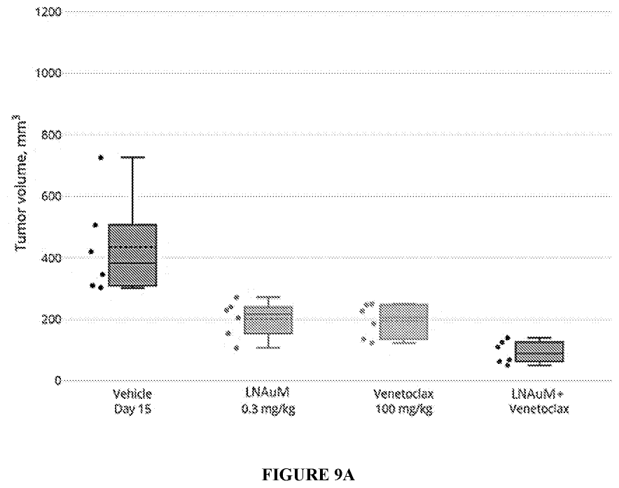

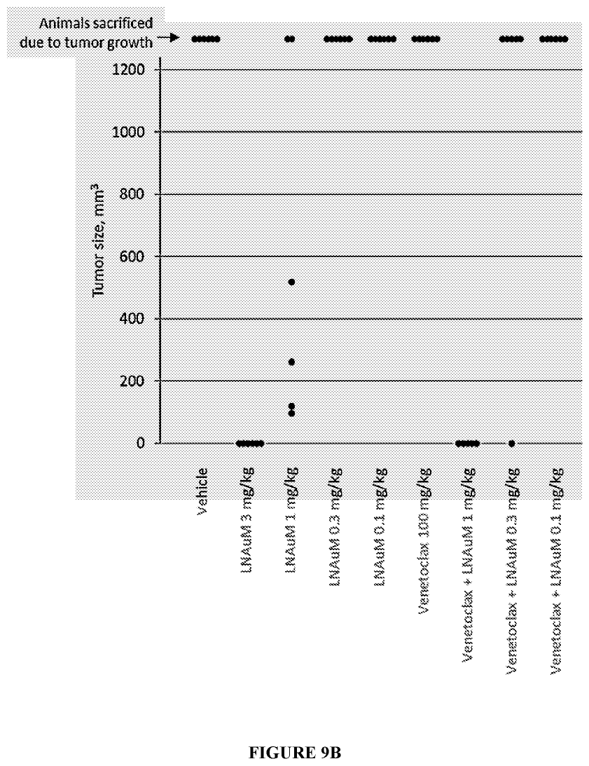

FIGS. 8-9 show the results of the study. In addition, with LNAuM single dose of 3 mg/kg all tumors (6/6) disappeared and did not regrow during the course of the whole study, and both 10 mg/kg single dose of lintuzumab naked antibody and non-binding IgG1 ADC control at single dose of 3 mg/kg showed only modest tumor growth inhibition (data not shown). The tumors grew fast in the control group, whereas both venetoclax and LNAuM treatments at all doses inhibited tumor growth.

Compared to either venetoclax or LNAuM 0.1 mg/kg treatments, the combination of these both (venetoclax+LNAuM 0.1 mg/kg) resulted in slower tumor growth compared to either of the single compound treatments (FIG. 8A).

As shown in FIG. 8B, the combination effect was even more prominent when the dose of the ADC was increased to 0.3 mg/kg: whereas both venetoclax or LNAuM 0.3 mg/kg single compound treatments showed comparable inhibition of tumor growth compared to vehicle only, the combination of these both (venetoclax+LNAuM 0.3 mg/kg) resulted in decreasing of the average tumor size up to day 15 after tumor inoculation (day 7 after start of treatment). Table below and FIG. 9A show tumor sizes of individual mice at day 15 after tumor inoculation. The Table also shows that the tumor growth index (TGI) was markedly lower in the combination group than in either single compound treatment group.

TABLE 4
Tumor sizes at day 15 after tumor inoculation of Vehicle,
LNAuM 0.3 mg/kg single dose, venetoclax 100 mg/kg
and the combination of the latter two groups:

| | | LNAuM | | |
|---|---|---|---|---|
| | Vehicle | 0.3 mg/kg | Venetoclax | Combination |
| | 506.626 | 106.624 | 122.793 | 67.626 |
| | 308.9295 | 271.472 | 226.1475 | 62.5 |
| | 420.25 | 204.723 | 134.946 | 50.336 |
| | 302.58 | 229.992 | 184.96 | 139.5375 |
| | 726 | 239.704 | 249.64 | 124.93 |
| | 345.6 | 153.6 | 246.895 | 109.8 |
| Average tumor size, | 435 | 201 | 194 | 92 |
| mm³ | | | | |
| TGI (average tumor | 1 | 0.46 | 0.45 | 0.21 |
| size compared | | | | |
| to Vehicle) | | | | |

As shown in FIG. 8C, the combination effect was prominent when the dose of the ADC was increased to 1 mg/kg. The tumors were eradicated in all the animals in the venetoclax+LNAuM 1 mg/kg combination group (5/5) during the study. Therefore, in FIG. 9B showing the comparison of all the study groups, a marked difference between the single compound treatments and the combination can be observed at day 54 after tumor inoculation i.e. the end of the study: 1) in venetoclax treatment group, all animals were sacrificed due to tumor growth during the study, 2) in LNAuM 1 mg/kg treatment group, two animals were sacrificed due to tumor growth, and in the rest of the animals there was a palpable tumor, and 3) in the venetoclax+LNAuM 1 mg/kg combination group, in all the 5 animals there was no palpable tumor i.e. the tumors were eradicated (5/5 complete response). Thus, the combination treatment resulted in cures whereas either of the single treatment did not result in any cures.

Taken together, both venetoclax and LNAuM showed anti-leukemia efficacy, no toxicity of either treatment was detected, and the combination of both venetoclax and LNAuM significantly improved the anti-leukemia efficacy compared to either of the single compound treatments.

### Example 5—Ex Vivo Study

### Patient Material

Six AML patient samples were obtained from the Helsinki University Hospital Comprehensive Cancer Center after informed consent and in compliance with the Declaration of Helsinki. Mononuclear cells were isolated with Ficoll gradient centrifugation and frozen in FBS with 5% DMSO. Samples used in the assay are described in Table 5-1.

The control cell lines were used to ensure that the compounds exhibit consistent behaviour, aligning with prior descriptions. The Daudi cell line (CD33neg) was used as a negative control, and the HL-60 cell line (CD33pos) as a positive control.

TABLE 5-1
Patient sample characteristics.

| | | Previous | Complex | | Response | Blast | CD33 |
|---|---|---|---|---|---|---|---|
| Patient | Diagnosis | treatment | karyotype | Mutations | to Ven/Aza | % | status |
| AML1 | Relapse | CytA/Ida, | No | NPM1, DNMT3, | RD | 34 | pos. |
| | AML | allo | | TET2 | | | |
| AML2 | Relapse | Aza | No | CEBPA, CEBPA | RD | 70 | pos. |
| | AML | | | | | | |
| AML3 | sAML | Aza | No | TET2, SRSF2, | CR | 70 | broad |
| | (KMML) | | | IDH2, CSFR3, | | | |
| | | | | CALR, RUNX1, | | | |
| | | | | CEBPA | | | |
| AML4 | De novo | None | No | DNMT3A, | CR | 41 | dim |
| | AML | | | RUNX1, | | | |
| | | | | U2AF1, FLT3- | | | |
| | | | | TKD | | | |
| AML5 | Relapse | CytA/Ida | No | TET2, SMC3A | MLFS | 90 | pos. |
| | AML | | | | | | |
| AML6 | Relapse | Ven/Aza | No | NPM1, TET2, | Ven relapse | 80 | pos. |
| | AML | | | FLT3-TKD | | | |

### Assays Using Flow Cytometry

AML cells were rapidly thawed in a 37° C. water bath and treated with Denarase (250 U/μl). The cells were cultured in SFEM II medium (STEMCELL Technologies) supplemented with 20 ng/ml SCF, 20 ng/ml TPO, and 20 ng/ml FLT3-L. The compounds were added the same day to 96-well conical bottom plates at seven different concentrations using an acoustic liquid handling device (Echo 550, Labcyte Inc), on top of 10 μl of culturing medium/well. A total of 100,000 cells were seeded per well in a volume of 100 μl. After 5 days, cell viability was analyzed using flow cytometry (iQue Screener PLUS instrument, Sartorius) as described previously (Kuusanmäki et al. 2020 Haematologica 105:708-20). To stain the cells, 25 μl antibody mixture containing CD45 (clone HI30), CD34 (8G12), CD117 (104D2), CD33 (WM53), and CD3 (UCHT1) antibodies in RPMI+10% FBS was added to the pelleted cells. All antibodies were obtained from BD biosciences and used at a dilution of 1:100, except CD3 in 1:50. Following a 20-minute incubation with the antibodies, the cells were centrifuged, and 25 μl of Annexin-V and 7-AAD mixture, both diluted 1:50 in Annexin V binding buffer, were added to each well. After a 10-minute incubation, the total number of viable blasts (CD34 or CD117), lymphocytes (CD3) and CD33-positive cells in each well was counted using Forecyte software (Sartorius) and normalized to DMSO control containing wells. The data was visualized using nonlinear regression analysis (GraphPad) and absolute IC50 values were calculated (50% reduction in cell viability compared to the control).

### Evaluation of LNAuM Combinations

AML cells were seeded onto 384-well drug combination plates in the SFEMII medium supplemented with cytokines at a volume of 25 μl per well (10 000 cells per well). Drug plates contained 8×8 combination matrices of LNAuM combined with either venetoclax, gilteritinib, or cytarabine. Each matrix was comprised of seven different concentrations of each drug and a DMSO and BzCl control well. After 4 days, cell viability was measured using the CellTiter-Glo reagent (Promega). The synergy effect was quantified using the zero-interaction potency (ZIP) model, utilizing the SynergyFinder web-based tool (Ianevski et al. 2022 Nucleic Acid Research Gkac382).

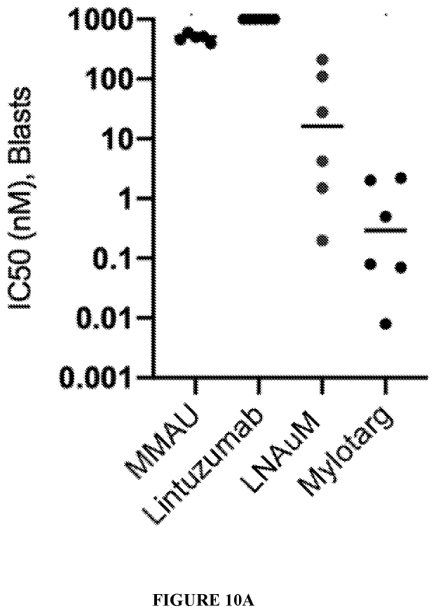

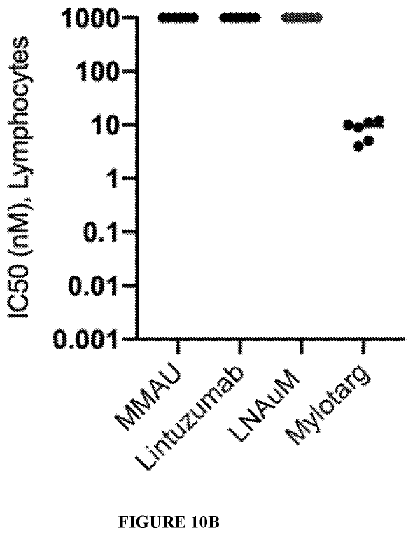

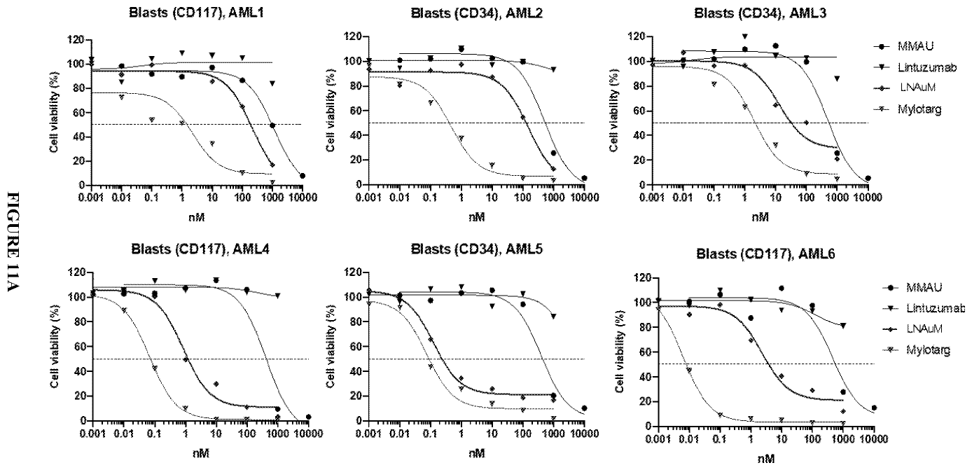

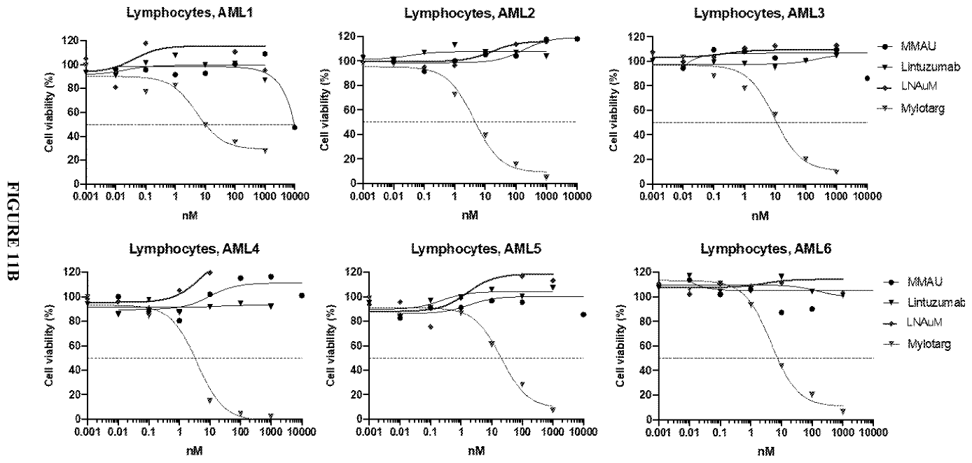

The AML samples were exposed to LNAuM, lintuzumab, MMAU, or gemtuzumab ozogamicin (GO; Mylotarg, Pfizer) for five days, after which remaining viable blast and lymphocytes were quantified using flow cytometry. LNAuM effectively targeted blasts with a median IC50 of 16 nM, while lintuzumab and MMAU displayed modest or no activity (FIG. 10A). GO demonstrated high efficacy against blasts, with a median IC50 of 0.3 nM (FIG. 10A). Subsequently, we assessed toxicity against lymphocytes (CD33-negative) in the same samples. LNAuM, LN, and MMAU did not exhibit cytotoxic activity against lymphocytes, whereas GO displayed significant lymphocyte toxicity across all samples (median IC50 of 9 nM; FIG. 10B). Individual dose response curves for each sample are presented in FIG. 11. These findings highlight LNAuM's potent activity against patient-derived blasts without off-target lymphocyte toxicity, contrasting with GO.

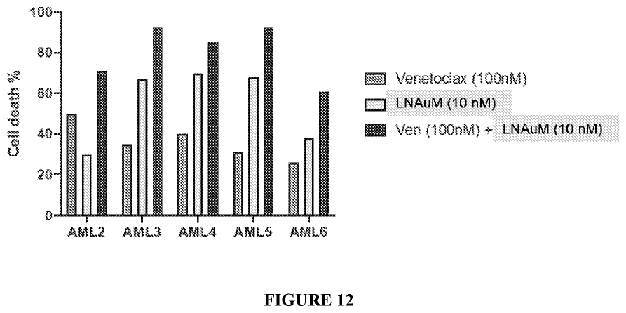

The LNAuM combinations were assessed in 8×8 matrices involving LNAuM, gilteritinib, venetoclax, and cytarabine (Table 5-2). Cell viability was measured using CTG after four days. To illustrate the most effective concentration ranges, the two lowest concentrations of each drug were removed from the plots. Venetoclax demonstrated the highest synergy with LNAuM, with an average synergy of 16.9 (ZIP, most synergistic area score: Table 5-2). Cytarabine exhibited high synergy in individual samples (FIG. 12). The FLT3 inhibitor gilteritinib showed positive synergy scores in the tested samples including two samples with FLT3-TKD mutations.

TABLE 5-2
Most synergistic area score (ZIP) of the tested samples.

| | LNAuM combination, | | | | |
|---|---|---|---|---|---|
| | most synergistic area score (ZIP) | | | | |
| Sample | +Venetoclax | +Gilterinib | +Cytarabine | Mutations | Diagnosis |
| AML2 | 10.2 | 9.6 | 11.7 | CEBPA, CEBPA | Relapse AML |
| AML3 | 22.4 | 2.9 | 11 | TET2, SRSF2, IDH2, | sAML (KMML) |
| | | | | RUNX1, CEBPA | |
| AML4 | 11.9 | 7.9 | 4.7 | DNMT3A, RUNX1, | De novo AML |
| | | | | U2AF1, FLT3-TKD | |
| AML5 | 27.6 | 1.5 | 18.4 | TET2, SMC3A | Relapse AML |
| AML6 | 12.4 | 0.64 | 1 | NPM1, TET2, FLT3-TKD | Relapse AML |
| Average | 16.9 | 4.5 | 9.4 | | |

What is claimed is:

1. A method of treating CD33 expressing cancer in a subject comprising administering to the subject an effective amount of chemotherapeutic agent and an effective amount of LNAuM of Formula (I): 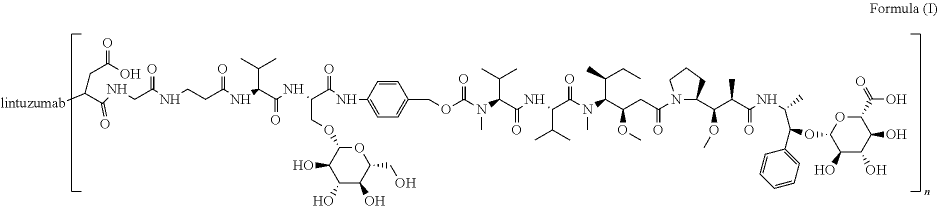
wherein
lintuzumab comprises a light chain having the amino acid sequence set forth in SEQ ID NO: 1 and a heavy chain having the amino acid sequence set forth in SEQ ID NO:2, and
n is an integer from 1 to 20.

2. The method of claim 1, wherein the cancer is a hematologic cancer.

3. The method of claim 2, wherein the hematologic cancer is leukemia.

4. The method of claim 3, wherein the leukemia is selected from the group consisting of acute myeloid leukemia (AML), chronic myeloid leukemia (CML), acute lymphoblastic leukemia (ALL), myelodysplastic syndrome (MDS), and acute promyelocytic leukaemia (APL).

5. The method of claim 4, wherein the cancer is acute myeloid leukemia (AML).

6. The method of claim 5, wherein the acute myeloid leukemia (AML) is refractory or relapse acute myeloid leukemia.

7. The method of claim 5 or 6, wherein the acute myeloid leukemia (AML) is characterized by FLT3 internal tandem duplication (FLT3-ITD).

8. The method of any preceding claim, wherein the chemotherapeutic agent is selected from the group consisting of an FLT3 inhibitor and a BCL2 inhibitor.

9. The method of claim 8, wherein the FLT3 inhibitor is selected from the group consisting of midostaurin and gilteritinib fumarate.

10. The method of claim 8, wherein the BCL2 inhibitor is venetoclax.

11. The method of claim 8, wherein the chemotherapeutic agent is selected from the group consisting of cytarabine and daunorubicin.

12. The method of any preceding claim, wherein the chemotherapeutic agent is venetoclax.

13. The method of claim 12, wherein venetoclax is administered at a daily dose of at least 50 mg, at least 70 mg, at least 100 mg, or at least 200 mg.

14. The method of any preceding claim, wherein the chemotherapeutic agent is cytarabine.

15. The method of claim 14, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

16. The method of any preceding claim, wherein the chemotherapeutic agent is midostaurin.

17. The method of claim 16, wherein midostaurin is administered daily at least a dose of 30 mg/m², 45 mg/m² or 60 mg/m².

18. The method of any preceding claim, wherein the chemotherapeutic agent is gilteritinib.

19. The method of claim 18, wherein gilteritinib is administered at a dose at least about 5 mg/day, at least about 10 mg/day, at least about 40 mg/day, or at least about 80 mg/day, or at least about 120 mg/day.

20. The method of any preceding claim, wherein the chemotherapeutic agent is daunorubicin.

21. The method of claim 20, wherein daunorubicin is administered at a dose at least about 10 mg/m²/day, at least about 30 mg/m²/day, at least about 60 mg/m²/day, or at least about 90 mg/m²/day.

22. The method of any preceding claim, wherein the LNAuM is administered at a concentration of at least about 0.1 mg/kg, at least about 0.3 mg/kg, at least about 0.5 mg/kg, at least about 0.8 mg/kg, at least about 1 mg/kg, at least about 1.5 mg/kg, at least about 2 mg/kg, at least about 2.5 mg/kg, at least about 3 mg/kg, at least about 3.5 mg/kg, or at least about 4 mg/kg.

23. A method of treating CD33 expressing acute myeloid leukemia (AML) in a subject comprising a step of administering cytarabine and LNAuM of Formula (I), 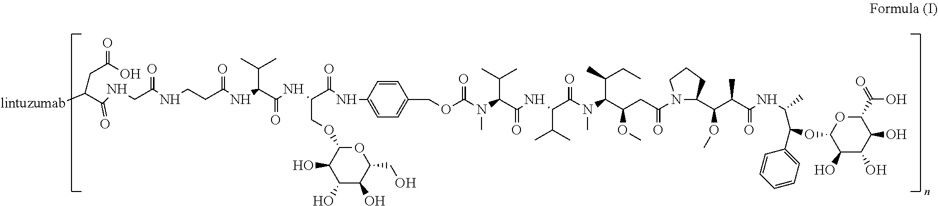
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

24. A method of treating CD33 expressing acute myeloid leukemia (AML) in a subject comprising the step of administering venetoclax and LNAuM of Formula (I), 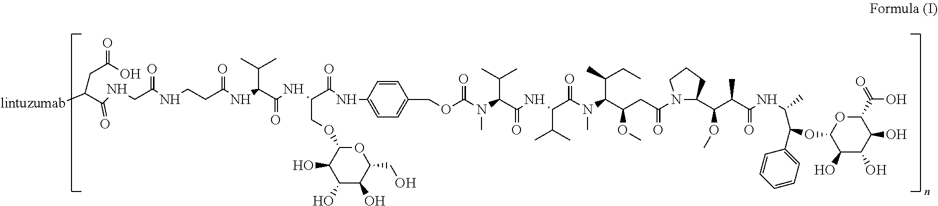
wherein venetoclax is administered at a daily dose of least 10 mg, at least 20 mg, at least 50 mg, at least 100 mg, at least 200 mg or at least 400 mg; and
n is an integer from 1 to 10.

25. A method of treating CD33 expressing acute myeloid leukemia (AML) in a subject comprising the step of administering midostaurin and LNAuM of Formula (I), 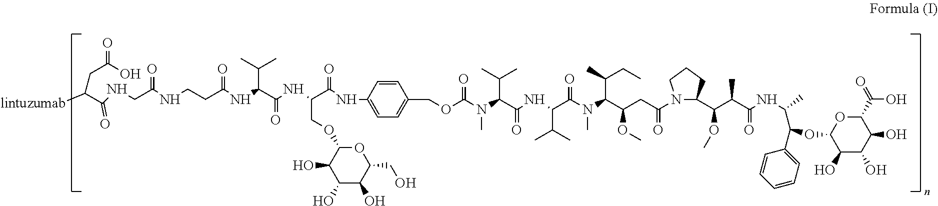
wherein midostaurin is administered daily at least a dose of 30 mg/m², 45 mg/m² or 60 mg/m²; and
n is an integer from 1 to 10.

26. A method of treating CD33 expressing acute myeloid leukemia (AML) in a subject comprising the step of administering gilteritinib and LNAuM of Formula (I), 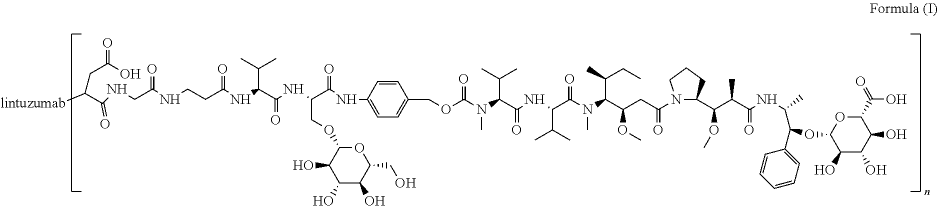
wherein gilteritinib is administered at a dose at least about 5 mg/day, at least about 10 mg/day, at least about 40 mg/day, or at least about 80 mg/day, or at least about 120 mg/day; and
n is an integer from 1 to 10.

27. A method of treating CD33 expressing acute myeloid leukemia (AML) in a subject comprising a step of administering daunorubicin and LNAuM of Formula (I), 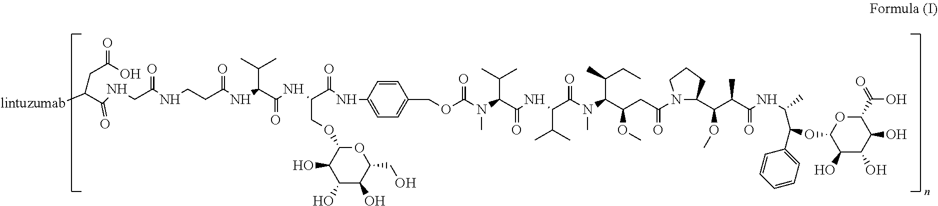
wherein daunorubicin is administered at a dose at least about 10 mg/m²/day, at least about 30 mg/m²/day, at least about 60 mg/m²/day, or at least about 90 mg/m²/day; and
n is an integer from 1 to 10.

28. A method of treating CD33 expressing acute myeloid leukemia (AML) in a subject comprising a step of administering cytarabine, venetoclax, and LNAuM of Formula (I), 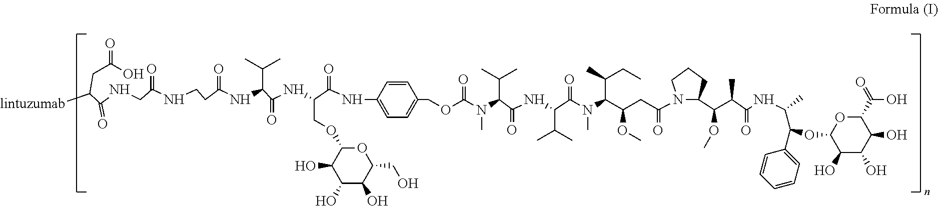
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered venetoclax is administered at a daily dose of at least 10 mg, at least 20 mg, at least 50 mg, at least 100 mg, at least 200 mg or at least 400 mg; and
n is an integer from 1 to 10.

29. LNAuM of Formula (I) 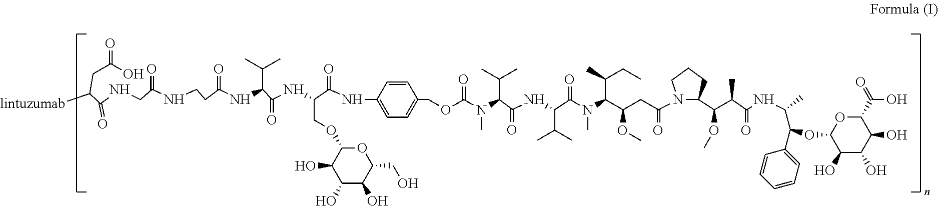
for use in a method for treating CD33 expressing cancer in a subject, wherein the method comprises administering to the subject an effective amount of the LNAuM and an effective amount of a chemotherapeutic agent, and wherein
lintuzumab comprises a light chain having the amino acid sequence set forth in SEQ ID NO: 1 and a heavy chain having the amino acid sequence set forth in SEQ ID NO:2, and
n is an integer from 1 to 20.

30. The LNAuM for use according to claim 29, wherein the cancer is a hematologic cancer.

31. The LNAuM for use according to claim 30, wherein the hematologic cancer is leukemia.

32. The LNAuM for use according to claim 31, wherein the leukemia is selected from the group consisting of acute myeloid leukemia (AML), chronic myeloid leukemia (CML), acute lymphoblastic leukemia (ALL), myelodysplastic syndrome (MDS), and acute promyelocytic leukaemia (APL).

33. The LNAuM for use according to any one of claims 29-32, wherein the cancer is acute myeloid leukemia (AML).

34. The LNAuM for use according to claim 33, wherein the acute myeloid leukemia (AML) is refractory or relapse acute myeloid leukemia.

35. The LNAuM for use according to claim 33 or 34, wherein the acute myeloid leukemia (AML) is characterized by FLT3 internal tandem duplication (FLT3-ITD).

36. The LNAuM for use according to any one of claims 29-35, wherein the chemotherapeutic agent is selected from the group consisting of an FLT3 inhibitor and a BCL2 inhibitor.

37. The LNAuM for use according to claim 36, wherein the FLT3 inhibitor is selected from the group consisting of midostaurin and gilteritinib fumarate.

38. The LNAuM for use according to claim 36 or 37, wherein the BCL2 inhibitor is venetoclax.

39. The LNAuM for use according to any one of claims 29-35, wherein the chemotherapeutic agent is selected from the group consisting of cytarabine and daunorubicin.

40. The LNAuM for use according to any one of claims 29-38, wherein the chemotherapeutic agent is venetoclax.

41. The LNAuM for use according to claim 38 or 40, wherein venetoclax is administered at a daily dose of at least 50 mg, at least 70 mg, at least 100 mg, or at least 200 mg.

42. The LNAuM for use according to any one of claims 29-39, wherein the chemotherapeutic agent is cytarabine.

43. The LNAuM for use according to claim 42, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

44. The LNAuM for use according to any one of claims 29-37, wherein the chemotherapeutic agent is midostaurin.

45. The LNAuM for use according to claim 44, wherein midostaurin is administered daily at least a dose of 30 mg/m², 45 mg/m² or 60 mg/m².

46. The LNAuM for use according to any one of claims 29-37, wherein the chemotherapeutic agent is gilteritinib.

47. The LNAuM for use according to claim 46, wherein gilteritinib is administered at a dose at least about 5 mg/day, at least about 10 mg/day, at least about 40 mg/day, or at least about 80 mg/day, or at least about 120 mg/day.

48. The LNAuM for use according to any one of claims 29-39, wherein the chemotherapeutic agent is daunorubicin.

49. The LNAuM for use according to claim 48, wherein daunorubicin is administered at a dose at least about 10 mg/m²/day, at least about 30 mg/m²/day, at least about 60 mg/m²/day, or at least about 90 mg/m²/day.

50. The LNAuM for use according to any one of claims 29-49, wherein the LNAuM is administered at a concentration of at least about 0.1 mg/kg, at least about 0.3 mg/kg, at least about 0.5 mg/kg, at least about 0.8 mg/kg, at least about 1 mg/kg, at least about 1.5 mg/kg, at least about 2 mg/kg, at least about 2.5 mg/kg, at least about 3 mg/kg, at least about 3.5 mg/kg, or at least about 4 mg/kg.

51. LNAuM of Formula (I) for use in a method for treating CD33 expressing acute myeloid leukemia (AML) in a subject 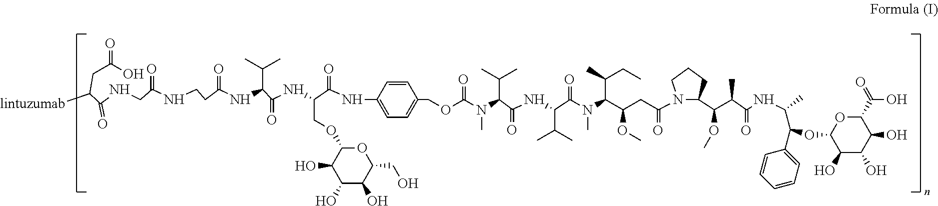
wherein the method comprises a step of administering cytarabine and the LNAuM, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

52. LNAuM of Formula (I) for use in a method for treating CD33 expressing acute myeloid leukemia (AML) in a subject 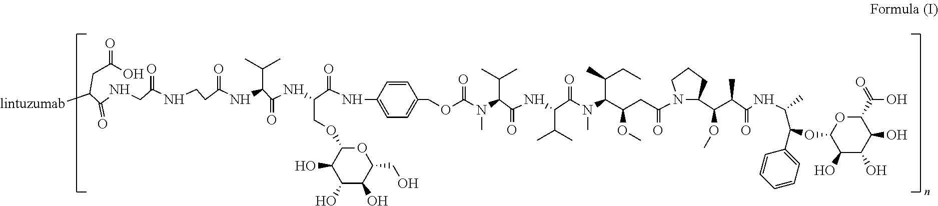
wherein the method comprises the step of administering venetoclax and the LNAuM, wherein venetoclax is administered at a daily dose of least 10 mg, at least 20 mg, at least 50 mg, at least 100 mg, at least 200 mg or at least 400 mg; and
n is an integer from 1 to 10.

53. LNAuM of Formula (I) for use in a method for treating CD33 expressing acute myeloid leukemia (AML) in a subject 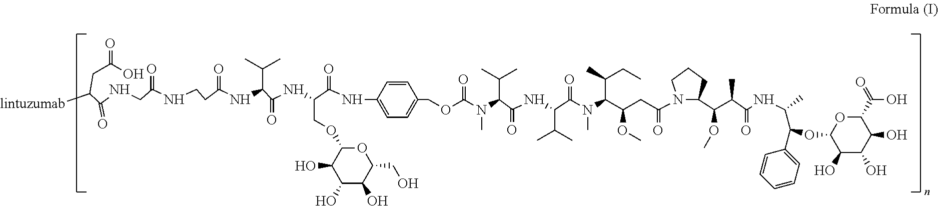
wherein the method comprises the step of administering midostaurin and the LNAuM, wherein midostaurin is administered daily at least a dose of 30 mg/m², 45 mg/m² or 60 mg/m²; and
n is an integer from 1 to 10.

54. LNAuM of Formula (I) for use in a method for treating CD33 expressing acute myeloid leukemia (AML) in a subject 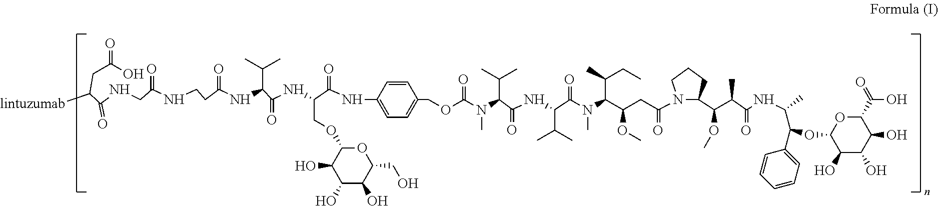
wherein the method comprises the step of administering gilteritinib and the LNAuM, wherein gilteritinib is administered at a dose at least about 5 mg/day, at least about 10 mg/day, at least about 40 mg/day, or at least about 80 mg/day, or at least about 120 mg/day; and
n is an integer from 1 to 10.

55. LNAuM of Formula (I) for use in a method for treating CD33 expressing acute myeloid leukemia (AML) in a subject 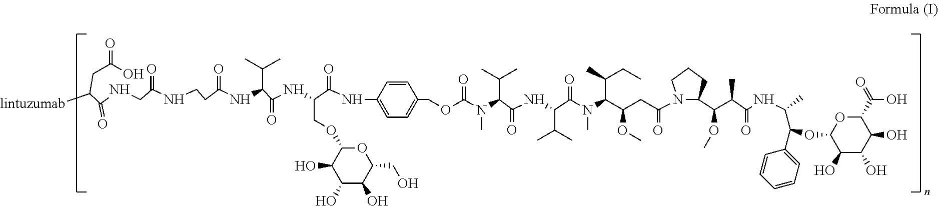
wherein the method comprises a step of administering daunorubicin and the LNAuM,
wherein daunorubicin is administered at a dose at least about 10 mg/m²/day, at least about 30 mg/m²/day, at least about 60 mg/m²/day, or at least about 90 mg/m²/day; and
n is an integer from 1 to 10.

56. LNAuM of Formula (I) for use in a method for treating CD33 expressing acute myeloid leukemia (AML) in a subject 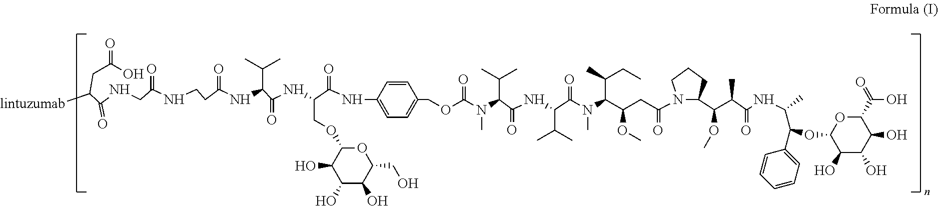
wherein the method comprises a step of administering cytarabine, venetoclax, and the LNAuM, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered at a daily dose of at least 10 mg, at least 20 mg, at least 50 mg, at least 100 mg, at least 200 mg or at least 400 mg; and
n is an integer from 1 to 10.

57. LNAuM of Formula (I) 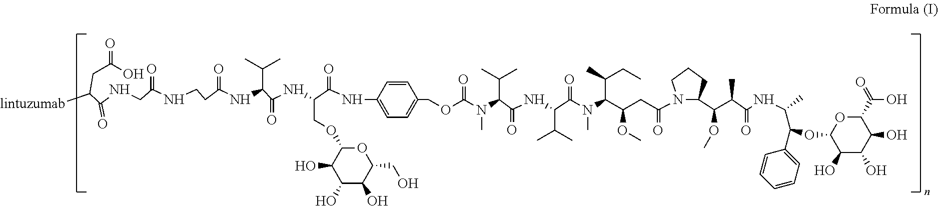
and a chemotherapeutic agent as a combination for use in a method for treating CD33 expressing cancer in a subject, wherein the method comprises administering to the subject an effective amount of the LNAuM and an effective amount of the chemotherapeutic agent, and wherein
lintuzumab comprises a light chain having the amino acid sequence set forth in SEQ ID NO: 1 and a heavy chain having the amino acid sequence set forth in SEQ ID NO:2, and
n is an integer from 1 to 20.

58. The LNAuM and the chemotherapeutic agent as a combination for use according to claim 57, wherein the cancer is a hematologic cancer.

59. The LNAuM and the chemotherapeutic agent as a combination for use according to claim 58, wherein the hematologic cancer is leukemia.

60. The LNAuM and the chemotherapeutic agent as a combination for use according to claim 59, wherein the leukemia is selected from the group consisting of acute myeloid leukemia (AML), chronic myeloid leukemia (CML), acute lymphoblastic leukemia (ALL), myelodysplastic syndrome (MDS), and acute promyelocytic leukaemia (APL).

61. The LNAuM and the chemotherapeutic agent as a combination for use according to any one of claims 57-60, wherein the cancer is acute myeloid leukemia (AML).

62. The LNAuM and the chemotherapeutic agent as a combination for use according to claim 61, wherein the acute myeloid leukemia (AML) is refractory or relapse acute myeloid leukemia.

63. The LNAuM and the chemotherapeutic agent as a combination for use according to claim 61 or 62, wherein the acute myeloid leukemia (AML) is characterized by FLT3 internal tandem duplication (FLT3-ITD).

64. The LNAuM and the chemotherapeutic agent as a combination for use according to any one of claims 57-63, wherein the chemotherapeutic agent is selected from the group consisting of an FLT3 inhibitor and a BCL2 inhibitor.

65. The LNAuM and the chemotherapeutic agent as a combination for use according to claim 64, wherein the FLT3 inhibitor is selected from the group consisting of midostaurin and gilteritinib fumarate.

66. The LNAuM and the chemotherapeutic agent as a combination for use according to claim 64 or 65, wherein the BCL2 inhibitor is venetoclax.

67. The LNAuM and the chemotherapeutic agent as a combination for use according to any one of claims 57-63, wherein the chemotherapeutic agent is selected from the group consisting of cytarabine and daunorubicin.

68. The LNAuM and the chemotherapeutic agent as a combination for use according to any one of claims 57-66, wherein the chemotherapeutic agent is venetoclax.

69. The LNAuM and the chemotherapeutic agent as a combination for use according to claim 66 or 68, wherein venetoclax is administered at a daily dose of at least 50 mg, at least 70 mg, at least 100 mg, or at least 200 mg.

70. The LNAuM and the chemotherapeutic agent as a combination for use according to any one of claims 57-63, wherein the chemotherapeutic agent is cytarabine.

71. The LNAuM and the chemotherapeutic agent as a combination for use according to claim 70, wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day.

72. The LNAuM and the chemotherapeutic agent as a combination for use according to any one of claims 57-65, wherein the chemotherapeutic agent is midostaurin.

73. The LNAuM and the chemotherapeutic agent as a combination for use according to claim 72, wherein midostaurin is administered daily at least a dose of 30 mg/m², 45 mg/m² or 60 mg/m².

74. The LNAuM and the chemotherapeutic agent as a combination for use according to any one of claims 57-65, wherein the chemotherapeutic agent is gilteritinib.

75. The LNAuM and the chemotherapeutic agent as a combination for use according to claim 74, wherein gilteritinib is administered at a dose at least about 5 mg/day, at least about 10 mg/day, at least about 40 mg/day, or at least about 80 mg/day, or at least about 120 mg/day.

76. The LNAuM and the chemotherapeutic agent as a combination for use according to any one of claims 57-67, wherein the chemotherapeutic agent is daunorubicin.

77. The LNAuM and the chemotherapeutic agent as a combination for use according to claim 76, wherein daunorubicin is administered at a dose at least about 10 mg/m²/day, at least about 30 mg/m²/day, at least about 60 mg/m²/day, or at least about 90 mg/m²/day.

78. The LNAuM and the chemotherapeutic agent as a combination for use according to any one of claims 57-77, wherein the LNAuM is administered at a concentration of at least about 0.1 mg/kg, at least about 0.3 mg/kg, at least about 0.5 mg/kg, at least about 0.8 mg/kg, at least about 1 mg/kg, at least about 1.5 mg/kg, at least about 2 mg/kg, at least about 2.5 mg/kg, at least about 3 mg/kg, at least about 3.5 mg/kg, or at least about 4 mg/kg.

79. LNAuM of Formula (I) and cytarabine as a combination for use in a method for treating CD33 expressing acute myeloid leukemia (AML) in a subject 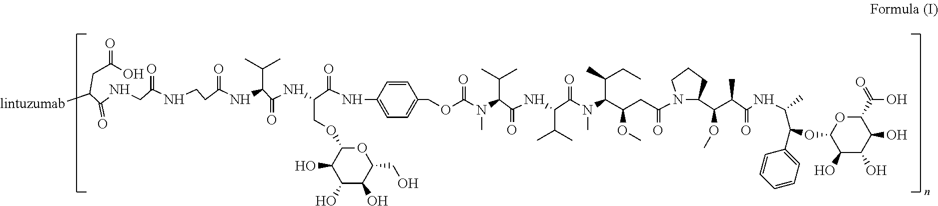
wherein the method comprises a step of administering cytarabine and the LNAuM,
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day; and
n is an integer from 1 to 10.

80. LNAuM of Formula (I) and venetoclax as a combination for use in a method for treating CD33 expressing acute myeloid leukemia (AML) in a subject 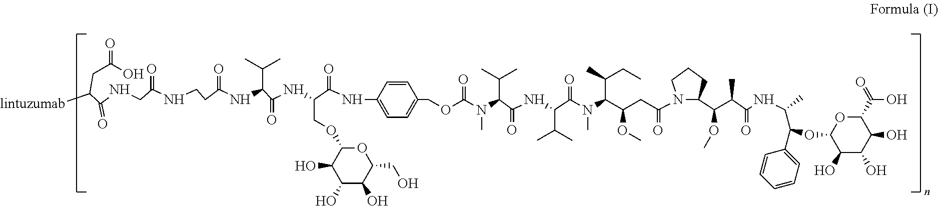
wherein the method comprises the step of administering venetoclax and the LNAuM,
wherein venetoclax is administered at a daily dose of least 10 mg, at least 20 mg, at least 50 mg, at least 100 mg, at least 200 mg or at least 400 mg; and
n is an integer from 1 to 10.

81. LNAuM of Formula (I) and midostaurin as a combination for use in a method for treating CD33 expressing acute myeloid leukemia (AML) in a subject 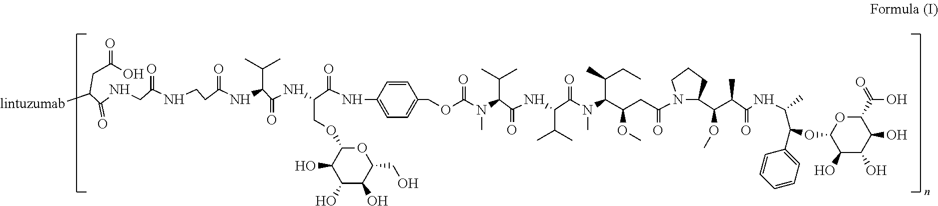
wherein the method comprises the step of administering midostaurin and the LNAuM,
wherein midostaurin is administered daily at least a dose of 30 mg/m², 45 mg/m² or 60 mg/m²; and
n is an integer from 1 to 10.

82. LNAuM of Formula (I) and gilteritinib as a combination for use in a method for treating CD33 expressing acute myeloid leukemia (AML) in a subject 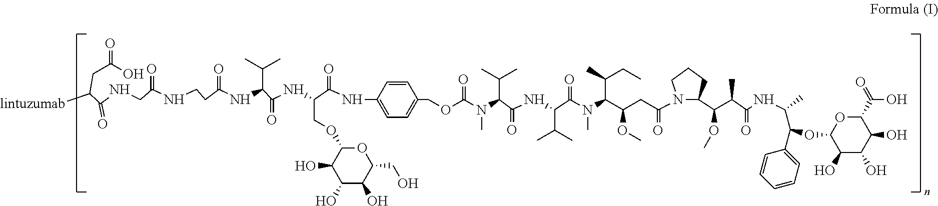
wherein the method comprises the step of administering gilteritinib and the LNAuM,
wherein gilteritinib is administered at a dose at least about 5 mg/day, at least about 10 mg/day, at least about 40 mg/day, or at least about 80 mg/day, or at least about 120 mg/day; and
n is an integer from 1 to 10.

83. LNAuM of Formula (I) and daunorubicin as a combination for use in a method for treating CD33 expressing acute myeloid leukemia (AML) in a subject 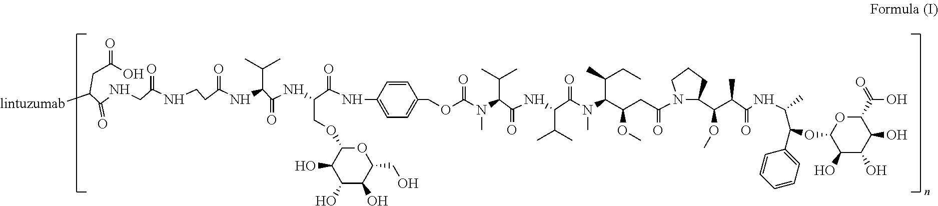
wherein the method comprises a step of administering daunorubicin and the LNAuM,
wherein daunorubicin is administered at a dose at least about 10 mg/m²/day, at least about 30 mg/m²/day, at least about 60 mg/m²/day, or at least about 90 mg/m²/day; and
n is an integer from 1 to 10.

84. LNAuM of Formula (I), cytarabine, and venetoclax as a combination for use in a method for treating CD33 expressing acute myeloid leukemia (AML) in a subject 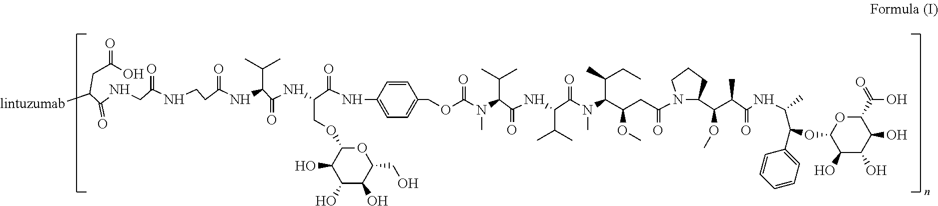
wherein the method comprises a step of administering cytarabine, venetoclax, and the LNAuM,
wherein cytarabine is administered at a dose of at least about 50 mg/m²/day, at least about 100 mg/m²/day, at least about 200 mg/m²/day, at least about 300 mg/m²/day, at least about 400 mg/m²/day, at least about 500 mg/m²/day, or at least about 1 g/m²/day;
wherein venetoclax is administered at a daily dose of at least 10 mg, at least 20 mg, at least 50 mg, at least 100 mg, at least 200 mg or at least 400 mg; and
n is an integer from 1 to 10.
